(19) United States
(12) Patent Application Publication
(10) Pub. No.: US 2024/0309428 A1
(43) Pub. Date: Sep. 19, 2024

(54) IMPACT OF TUMOR MICROENVIRONMENT ON EFFICACY OF IMMUNOTHERAPY

(71) Applicant: Kite Pharma, Inc., Foster City, CA (US)

(72) Inventors: Simone Filosto, Santa Monica, CA (US); Gayatri R. Tiwari, Camarillo, CA (US); Justin Szu-Ting Chou, Los Angeles, CA (US)

(21) Appl. No.: 18/604,854

(22) Filed: Mar. 14, 2024

Related U.S. Application Data
(60) Provisional application No. 63/502,295, filed on May 15, 2023.
(60) Provisional application No. 63/496,887, filed on Apr. 18, 2023.
(60) Provisional application No. 63/491,516, filed on Mar. 21, 2023.
(60) Provisional application No. 63/490,870, filed on Mar. 17, 2023.

(51) Int. Cl. C12Q 1/6809; A61K 35/17; A61K 39/00; A61P 35/00; C12Q 1/6886

(52) U.S. Cl. CPC C12Q 1/6809; A61K 35/17; A61K 39/4611; A61K 39/4631; A61K 39/4632; A61P 35/00; C12Q 1/6886; C12Q 2600/106; C12Q 2600/158

(57) ABSTRACT

The disclosure relates to methods of prognosis and therapy, compositions for immunotherapies, methods of improving said compositions, and immunotherapies using the same (e.g., T cells, non-T cells, TCR-based therapies, and CAR-based therapies).

### CROSS REFERENCE TO RELATED APPLICATIONS

This application claims priority to U.S. Provisional Patent Application No. 63/490,870, filed on Mar. 17, 2023; U.S. Provisional Patent Application No. 63/491,516, filed on Mar. 21, 2023; U.S. Provisional Patent Application No. 63/496,887, filed on Apr. 18, 2023; and U.S. Provisional Patent Application No. 63/502,295, filed on May 15, 2023, each of which is hereby incorporated in its entirety.

### FIELD

This disclosure relates to methods of diagnosis and prognosis of subjects undergoing immunotherapies, compositions for immunotherapies, and immunotherapies using the same.

### BACKGROUND

Axicabtagene ciloleucel (axi-cel) is an autologous anti-CD19 chimeric antigen receptor (CAR) T-cell therapy initially approved for the treatment of relapsed/refractory (R/R) large B-cell lymphoma (LBCL) in adults after ≥2 lines of systemic therapy. ZUMA-7 (NCT03391466) was the first randomized, global, multicenter phase 3 study of axi-cel versus historical standard of care (SOC) as second-line treatment in patients with R/R LBCL; SOC consisted of two or three cycles of protocol-defined, investigator-selected, platinum-based chemotherapy with intention to subsequently undergo high-dose chemotherapy with autologous stem-cell transplantation (HDT-ASCT) for chemosensitive patients. Axi-cel was superior to SOC with significant improvement in efficacy and a manageable safety profile. In the primary analysis, the event-free survival (EFS) hazard ratio (HR) was 0.398 (P<0.0001; median EFS of 8.3 versus 2.0 months and estimated 24-month EFS rates of 40.5% versus 16.3% in the axi-cel versus SOC arms, respectively). Despite these striking results, a substantial number of patients presented primary (no response) or secondary (relapse after initial response) resistance to CAR T-cell therapy, warranting further investigation into potential biomarkers associated with treatment resistance.

In LBCL, among clinical and real-world evidence in the chemoimmunotherapy era, known prognostic factors include high tumor burden, elevated lactate dehydrogenase (LDH), activated B-cell (ABC)-like molecular subgroup, age, and systemic inflammatory markers, like interleukin-6 (IL-6) and C-reactive protein (CRP). In the cellular therapy era, as shown in ZUMA-1 (third-line or higher LBCL), tumor burden and LDH associated negatively with efficacy to CAR T-cell therapy. Additionally, quality and quantity of pretreatment tumor infiltration of T cells, as characterized by ImmunoSign 21 (IS21; T-cell gene expression signature) and by Immunoscore (immunohistochemistry [IHC] with CD3 and CD8 cells) positively associated with outcomes to CAR T-cell therapy. Translational data from patients treated with CAR T-cell therapy in the real world further highlight the impact of tumor-associated chronic inflammation, checkpoint ligand upregulation, myeloid cell suppression of CAR T-cell function, and an association between patterns of tumor genomic complexity and CAR T-cell outcomes. Nonetheless, predictive biomarkers for CAR T-cell intervention across lines of therapy are not well established, and the associations between tumor gene expression profiles and responses to CAR T cells have not been exhaustively investigated. Furthermore, although the predictive and prognostic roles of the TME have been well described for solid tumors, the importance of the immune contexture within the tumor for CAR T-cell therapy remains elusive.

Toward addressing these needs, analyses were performed of pretreatment tumor characteristics in ZUMA-7 to discover tumor-specific features predictive of axi-cel or SOC efficacy.

### SUMMARY

It is to be understood that the disclosure is not limited in its application to the details set forth in the following embodiments, claims, description and figures. The disclosure is capable of other embodiments and of being practiced or carried out in numerous other ways.

Provided herein are methods that involve assessing particular parameters, e.g., expression of specific biomarkers or analytes, that can be correlated with an outcome, such as a therapeutic outcome, including a response, such as a complete response (CR) or a partial response (PR); or a safety outcome (e.g., an adverse event), such as a development of a toxicity, for example, neurotoxicity or CRS, after administration of immunotherapy (e.g., cell therapy). Also provided are methods to assess the likelihood of response and/or likelihood of risk of toxicity, based on assessment of the parameters, such as expression of biomarkers or analytes in the patient. Also provided are immunotherapies (e.g., T cells, non-T cells, TCR-based therapies, CAR-based therapies, bispecific T-cell engagers (BiTEs), and/or immune checkpoint blockade), including methods and uses of cells (e.g., engineered T cells) and/or compositions thereof, for the treatment of subjects having a disease or condition, which generally is or includes a cancer or a tumor, such as a leukemia or a lymphoma. In some aspects, the methods and uses provide for or achieve improved response and/or more durable responses or efficacy and/or a reduced risk of toxicity or other side effects, in subjects treated with some methods, as compared to certain alternative methods. In some embodiments, the methods comprise the administration of specified numbers or relative numbers of the engineered cells, the administration of defined ratios of particular types of the cells, treatment of particular patient populations, such as those having a particular risk profile, staging, and/or prior treatment history, administration of additional therapeutic agents and/or combinations thereof.

In one aspect, the disclosure relates to an immunotherapy product. By way of non-limiting example, one aspect of the disclosure relates to Yescarta as a second-line therapy. In certain aspects, without being bound by any particular theory, the primary overall survival (OS) analysis results of the Phase 3 ZUMA-7 study, in which Yescarta showed a statistically significant improvement in OS versus historical treatment, which was the standard of care (SOC) in a curative setting for nearly 30 years for second-line relapsed/refractory large B-cell lymphoma (R/R LBCL) within 12 months of completion of first-line therapy. This is a multi-step process involving platinum-based salvage combination chemoimmunotherapy regimen followed by high-dose therapy (HDT) and stem cell transplant (ASCT) in those who respond to salvage chemotherapy. OS was designated as a clinically important prespecified key secondary endpoint, defined as the length of time from randomization to death from any cause.

In one aspect, the disclosure provides a method of predicting a likelihood of a response to a cell therapy product in a patient in need thereof comprising: quantifying a gene expression level of at least one gene selected from the group consisting of CD19, MS4A1, TNFRSF17, BLK, FCRL2, FAM30A, PNOC, SPIB, TCL1A, BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2; and determining the likelihood of the response to the cell therapy product in the patient at least in part from the gene expression level. In such an embodiment, an increase in the gene expression level of the at least one gene as compared to a control value is indicative of an increased likelihood of a response or of a reduced likelihood of a response as compared to a predetermined likelihood of response rate, and the gene expression level is quantified from a patient sample, and the patient sample is collected from the patient prior to treatment with the cell therapy product.

In one aspect, the disclosure provides a method for treating a malignancy in a patient comprising: quantifying a gene expression level of at least one gene selected from the group consisting of CD19, MS4A1, TNFRSF17, BLK, FCRL2, FAM30A, PNOC, SPIB, TCL1A, BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2; determining whether the patient should be administered an effective dose of the cell therapy product as a second-line therapy, or an effective dose of the cell therapy product as a third-line therapy at least in part from the quantifying the gene expression level of at least one gene; and administering the effective dose of the cell therapy product as a second-line therapy or as a third-line therapy based on the determining step. In certain embodiments, the patient is administered the effective dose of the cell therapy product as a second-line therapy if the gene expression level of the at least one of CD19, MS4A1, TNFRSF17, BLK, FCRL2, FAM30A, PNOC, SPIB, and TCL1A is at or above a control value for the at least one gene, or the patient is administered the effective dose of the cell therapy product as a third-line therapy if the gene expression level of the at least one of CD19, MS4A1, TNFRSF17, BLK, FCRL2, FAM30A, PNOC, SPIB, and TCL1A is below a control value for the at least one gene, or the patient is administered the effective dose of the cell therapy product as a second-line therapy if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or below a control value for the at least one gene, or the patient is administered the effective dose of the cell therapy product as a third-line therapy if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or above a control value for the at least one gene.

In one embodiment, the immunotherapy is T cell therapy. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells In one embodiment, the immunotherapy is CAR T cell or TCR T cell therapy. In one embodiment, the immunotherapy is anti-CD19 CAR T cell therapy.

The following are non-limiting embodiments of the disclosure.

In at least a first aspect, the present disclosure provides a method of predicting a likelihood of a response to a cell therapy product in a patient in need thereof comprising: quantifying a gene expression level of at least one gene selected from the group consisting of CD19, MS4A1, TNFRSF17, BLK, FCRL2, FAM30A, PNOC, SPIB, TCL1A, BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2; and determining the likelihood of the response to the cell therapy product in the patient at least in part from the gene expression level, wherein an increase in the gene expression level of the at least one gene as compared to a control value is indicative of an increased likelihood of a response or of a reduced likelihood of a response as compared to a predetermined likelihood of response rate, and wherein the gene expression level is quantified from a patient sample, and the patient sample is collected from the patient prior to treatment with the cell therapy product.

In certain aspects, the at least one gene is selected from the group consisting of CD19, MS4A1, TNFRSF17, BLK, FCRL2, FAM30A, PNOC, SPIB, and TCL1A, and wherein an increase in the gene expression level of the at least one gene as compared to a control value is indicative of an increased likelihood of a response as compared to a predetermined likelihood of response rate.

In certain aspects, the at least one gene is selected from the group consisting of CD19, MS4A1, and TNFRSF17, and wherein an increase in the gene expression level of the at least one gene as compared to a control value is indicative of an increased likelihood of a response as compared to a predetermined likelihood of response rate.

In certain aspects, the at least one gene exhibits an increase of at least 20% in the expression level of CD19 versus a control expression level of CD19, an increase of at least 40% in the expression level of MS4A1 versus a control expression level of MS4A1, and an increase of at least 60% in the expression level of TNFRSF17 versus a control expression level of TNFRSF17, is indicative of an increased likelihood of a response as compared to a predetermined likelihood of response rate.

In certain aspects, the at least one gene is selected from the group consisting of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2, and wherein an increase in the gene expression level of the at least one gene as compared to a control value is indicative of a decreased likelihood of a response as compared to a predetermined likelihood of response rate.

In certain aspects, a response is defined as one or more of a complete response, a partial response, an ongoing response, a progression free survival, or an event free survival.

In certain aspects, the cell therapy product is CAR T or TCR T cell therapy that recognizes a target antigen.

In certain aspects, the cell therapy product is autologous or allogeneic.

In certain aspects, the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

In certain aspects, the cell therapy product expresses a chimeric antigen receptor comprising a CD28 co-stimulatory domain.

In certain aspects, the patient has been diagnosed with a cancer/tumor selected from the group consisting of a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

In certain aspects, the cancer is (relapsed or refractory) diffuse large B-cell lymphoma (DLBCL) not otherwise specified, primary mediastinal large B-cell lymphoma, high grade B-cell lymphoma (HGBL), DLBCL arising from follicular lymphoma, or mantle cell lymphoma.

In certain aspects, the cell therapy product is selected from axicabtagene ciloleucel, brexucabtagene autoleucel, tisagenlecleucel, lisocabtagene maraleucel, and bb2121.

In certain aspects, the cell therapy product is administered as a second-line therapy.

In certain aspects, the patient sample is a tumor biopsy.

In certain aspects, the present disclosure provides a method for treating a malignancy in a patient comprising:
quantifying a gene expression level of at least one gene selected from the group consisting of CD19, MS4A1, TNFRSF17, BLK, FCRL2, FAM30A, PNOC, SPIB, TCL1A, BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2;
determining whether the patient should be administered an effective dose of a cell therapy product as a second-line therapy, or an effective dose of a cell therapy product as a third-line therapy at least in part from the quantifying the gene expression level of at least one gene; and administering the effective dose of the cell therapy product as a second-line therapy or as a third-line therapy based on the determining step,
wherein the gene expression level is quantified from a patient sample, and the patient sample is collected from the patient prior to treatment with the cell therapy product,
wherein the patient is administered the effective dose of the cell therapy product as a second-line therapy if the gene expression level of the at least one of CD19, MS4A1, TNFRSF17, BLK, FCRL2, FAM30A, PNOC, SPIB, and TCL1A is at or above a control value for the at least one gene, or
wherein the patient is administered the effective dose of the cell therapy product as a third-line therapy if the gene expression level of the at least one of CD19, MS4A1, TNFRSF17, BLK, FCRL2, FAM30A, PNOC, SPIB, and TCL1A is at or below a control value for the at least one gene, or
wherein the patient is administered the effective dose of the cell therapy product as a second-line therapy if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or below a control value for the at least one gene, or
wherein the patient is administered the effective dose of the cell therapy product as a third-line therapy if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or above a control value for the at least one gene.

In certain aspects, the gene expression level of the at least one of CD19, MS4A1, TNFRSF17, BLK, FCRL2, FAM30A, PNOC, SPIB, and TCL1A is below a control value for the at least one gene, or if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or above a control value for the at least one gene, then the patient is administered a second-line course of therapy for the malignancy which does not comprise cell therapy.

In certain aspects, the at least one gene is selected from the group consisting of CD19, MS4A1, TNFRSF17, BLK, FCRL2, FAM30A, PNOC, SPIB, and TCL1A.

In certain aspects, the at least one gene is selected from the group consisting of CD19, MS4A1, and TNFRSF17.

In certain aspects, the cell therapy product is CAR T or TCR T cell therapy that recognizes a target antigen.

In certain aspects, the cell therapy product is autologous or allogeneic.

In certain aspects, the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

In certain aspects, the cell therapy product expresses a chimeric antigen receptor comprising a CD28 co-stimulatory domain.

In certain aspects, the patient has been diagnosed with a cancer/tumor selected from the group consisting of a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

In certain aspects, the cancer is (relapsed or refractory) diffuse large B-cell lymphoma (DLBCL) not otherwise specified, primary mediastinal large B-cell lymphoma, high grade B-cell lymphoma (HGBL), DLBCL arising from follicular lymphoma, or mantle cell lymphoma.

In certain aspects, the cell therapy product is selected from axicabtagene ciloleucel, brexucabtagene autoleucel, tisagenlecleucel, lisocabtagene maraleucel, and bb2121.

In certain aspects, the patient sample is a tumor biopsy.

In certain aspects, methods of treating a subject with lymphoma are disclosed comprising administering to the subject a debulking regimen; and administering to the subject an immunotherapy following the debulking regimen.

In certain aspects, the debulking regimen comprises any of the options included in Table 39. In certain aspects, the debulking regimen comprises at least 2 of the options included in Table 39.

In certain aspects, the immunotherapy comprises an anti-CD19 CAR T-cell.

In certain aspects, a subject is administered the debulking regimen when the subject has a tumor burden above a predetermined level. In certain aspects, the subject is not administered the debulking regimen when the subject has a tumor burden below a predetermined level.

In one aspect, the disclosure relates to a method of predicting a likelihood of response to a cell therapy product in a patient in need thereof including: quantifying a gene expression level of at least two genes selected a group consisting of CD19, MS4A1, TNFRSF17, BLK, FCRL2, FAM30A, PNOC, SPIB, and TCL1A; calculating a composite expression score comprising adding the gene expression levels of the at least two genes; and determining the likelihood of the response to the cell therapy product in the patient at least in part from the composite expression score. In such an embodiment, an increase in the composite expression score as compared to a control value is indicative of an increased likelihood of a response as compared to a predetermined likelihood of response rate. In such an embodiment, the composite expression scores are calculated as follows. The individual expression levels of each of the at least two genes from CD19, MS4A1, TNFRSF17, BLK, FCRL2, FAM30A, PNOC, SPIB, and TCL1A are measured. These individual expression levels are then normalized and averaged to generate a composite expression score for the at least two genes. Then, the generated composite expression score is compared to a control value, where the control value is the historical median of historical composite expression scores for the at least two genes from other patients.

In one aspect, the disclosure relates to a method of predicting a likelihood of response to a cell therapy product in a patient in need thereof including: quantifying a gene expression level of at least two genes selected a group consisting of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2; calculating a composite expression score comprising adding the gene expression levels of the at least two genes; and determining the likelihood of the response to the cell therapy product in the patient at least in part from the composite expression score. In such an embodiment, an increase in the composite expression score as compared to a control value is indicative of a decreased likelihood of a response as compared to a predetermined likelihood of response rate. In such an embodiment, the composite expression scores are calculated as follows. The individual expression levels of each of the at least two genes from BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 are measured. These individual expression levels are then normalized and averaged to generate a composite expression score for the at least two genes. Then, the generated composite expression score is compared to a control value, where the control value is the historical median of historical composite expression scores for the at least two genes from other patients.

In one aspect, the disclosure relates to a method for treating a malignancy in a patient comprising: quantifying a gene expression level of at least two genes selected from the group consisting of CD19, MS4A1, TNFRSF17, BLK, FCRL2, FAM30A, PNOC, SPIB, and TCL1A; calculating a composite expression score comprising adding the gene expression levels of the at least two genes; and determining whether the patient should be administered an effective dose of a cell therapy product as a second-line therapy, or an effective dose of a cell therapy product as a third-line therapy at least in part from the composite expression score; and administering the effective dose of the cell therapy product as a second-line therapy or as a third-line therapy based on the determining step. In such an embodiment, the patient is administered the effective dose of the cell therapy product as a second-line therapy if the composite expression score of the at least two of CD19, MS4A1, TNFRSF17, BLK, FCRL2, FAM30A, PNOC, SPIB, and TCL1A is at or above a control value, or the patient is administered the effective dose of the cell therapy product as a third-line therapy if the composite expression score of the at least two of CD19, MS4A1, TNFRSF17, BLK, FCRL2, FAM30A, PNOC, SPIB, and TCL1A is at or below a control value. In such an embodiment, the composite expression scores are calculated as follows. The individual expression levels of each of the at least two genes from CD19, MS4A1, TNFRSF17, BLK, FCRL2, FAM30A, PNOC, SPIB, and TCL1A are measured. These individual expression levels are then normalized and averaged to generate a composite expression score for the at least two genes. Then, the generated composite expression score is compared to a control value, where the control value is the historical median of historical composite expression scores for the at least two genes from other patients.

In one aspect, the disclosure relates to a method for treating a malignancy in a patient comprising: quantifying a gene expression level of at least two genes selected from the group consisting of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2; calculating a composite expression score comprising adding the gene expression levels of the at least two genes; and determining whether the patient should be administered an effective dose of a cell therapy product as a second-line therapy, or an effective dose of a cell therapy product as a third-line therapy at least in part from the composite expression score; and administering the effective dose of the cell therapy product as a second-line therapy or as a third-line therapy based on the determining step. In such an embodiment, the patient is administered the effective dose of the cell therapy product as a second-line therapy if the composite expression score of the at least two of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or below a control value, or the patient is administered the effective dose of the cell therapy product as a third-line therapy if the composite expression score of the at least two of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or above a control value. In such an embodiment, the composite expression scores are calculated as follows. The individual expression levels of each of the at least two genes from BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 are measured. These individual expression levels are then normalized and averaged to generate a composite expression score for the at least two genes. Then, the generated composite expression score is compared to a control value, where the control value is the historical median of historical composite expression scores for the at least two genes from other patients.

### DETAILED DESCRIPTION

The present disclosure is based in part on the discovery that pre-cell therapy treatment gene expression levels of CD19, MS4A1, TNFRSF17, BLK, FCRL2, FAM30A, PNOC, SPIB, and TCL1A are positively associated with an increased likelihood of response to a cell therapy, while pre-cell therapy treatment gene expression levels of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 are inversely associated with a likelihood of response to a cell therapy. The present findings inform whether a patient should be administered a cell therapy.

In one aspect, the disclosure relates to an immunotherapy product. By way of non-limiting example, one aspect of the disclosure relates to Yescarta as a second-line therapy. In certain aspects, without being bound by any particular theory, the primary overall survival (OS) analysis results of the Phase 3 ZUMA-7 study, in which Yescarta showed a statistically significant improvement in OS versus historical treatment, which was the standard of care (SOC) in a curative setting for nearly 30 years for second-line relapsed/refractory large B-cell lymphoma (R/R LBCL) within 12 months of completion of first-line therapy. This is a multi-step process involving platinum-based salvage combination chemoimmunotherapy regimen followed by high-dose therapy (HDT) and stem cell transplant (ASCT) in those who respond to salvage chemotherapy. OS was designated as a clinically important prespecified key secondary endpoint, defined as the length of time from randomization to death from any cause.

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Exemplary routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods. In one embodiment, the CAR T cell treatment is administered via an “infusion product” comprising CAR T cells.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e., expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocks a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

In one embodiment, the CAR T cell treatment comprises “axicabtagene ciloleucel treatment”. “Axicabtagene ciloleucel treatment” consists of a single infusion of anti-CD19 CAR transduced autologous T cells administered intravenously at a target dose of 2×10⁶ anti-CD19 CAR T cells/kg. For subjects weighing greater than 100 kg, a maximum flat dose of 2×10⁸ anti-CD19 CAR T cells may be administered. The anti-CD19 CAR T cells are autologous human T cells that have been engineered to express an extracellular single-chain variable fragment (scFv) with specificity for CD19 linked to an intracellular signaling part comprised of signaling domains from CD28 and CD3ζ (CD3-zeta) molecules arranged in tandem anti-CD19 CAR vector construct has been designed, optimized and initially tested at the Surgery Branch of the National Cancer Institute (NCI, IND 13871) (Kochenderfer et al, J Immunother. 2009; 32(7):689-702; Kochenderfer et al, Blood. 2010; 116(19):3875-86). The scFv is derived from the variable region of the anti-CD19 monoclonal antibody FMC63 (Nicholson et al, Molecular Immunology. 1997; 34(16-17):1157-65). A portion of the CD28 costimulatory molecule is added, as murine models suggest this is important for the anti-tumor effect and persistence of anti-CD19 CAR T cells (Kowolik et al, Cancer Res. 2006; 66(22):10995-1004). The signaling domain of the CD3-zeta chain is used for T cell activation. These fragments were cloned into the murine stem cell virus-based (MSGV1) vector, utilized to genetically engineer the autologous T cells. The CAR construct is inserted into the T cells' genome by retroviral vector transduction. Briefly, peripheral blood mononuclear cells (PBMCs) are obtained by leukapheresis and Ficoll separation. Peripheral blood mononuclear cells are activated by culturing with an anti-CD3 antibody in the presence of recombinant interleukin 2 (IL-2). Stimulated cells are transduced with a retroviral vector containing an anti-CD19 CAR gene and propagated in culture to generate sufficient engineered T cells for administration. In some embodiments, the CAR T cell therapy is Yescarta® (axicabtagene ciloleucel). In some embodiments, the CAR T cell therapy is Tecartus® (brexucabtagene autoleucel).

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-10 (MIP-1b), gamma-induced protein 10 (IP-10), and thymus and activation regulated chemokine (TARC or CCL17).

As used herein, “chimeric receptor” refers to an engineered surface expressed molecule capable of recognizing a particular molecule. Chimeric antigen receptors (CARs) and engineered T cell receptors (TCRs), which comprise binding domains capable of interacting with a particular tumor antigen, allow T cells to target and kill cancer cells that express the particular tumor antigen. In one embodiment, the T cell treatment is based on T cells engineered to express a chimeric antigen receptor (CAR) or a T cell receptor (TCR), which comprises (i) an antigen binding molecule, (ii) a costimulatory domain, and (iii) an activating domain. The costimulatory domain may comprise an extracellular domain, a transmembrane domain, and an intracellular domain, wherein the extracellular domain comprises a hinge domain, which may be truncated.

A “therapeutically effective amount,” “effective dose,” “effective amount,” or “therapeutically effective dosage” of a therapeutic agent, e.g., engineered CAR T cells, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g., animal model) systems.

The term “combination” refers to either a fixed combination in one dosage unit form, or a combined administration where a compound of the present disclosure and a combination partner (e.g., another drug as explained below, also referred to as “therapeutic agent” or “agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

As used throughout, a “control value” refers to a historical value of a particular analyte observed in a population prior to administration of a cellular therapeutic product. In some embodiments, deviations from the historical value are correlated with an increased or a decreased likelihood of a response to the cellular therapeutic product in a particular patient versus a predetermined and/or historical likelihood of a response to the cellular therapeutic product. More specifically, in some embodiments, an increased expression level of an analyte in a test sample from a patient versus a control expression level for that corresponding analyte is associated with an increased chance of response in that patient to the cellular therapeutic product versus a predetermined and/or historical likelihood of a response to the cellular therapeutic product. In certain embodiments, the increased chance of response is measured with respect to a known historical average likelihood of a response to the cellular therapeutic product in a population. In some embodiments, an increased expression level of an analyte in a test sample from a patient versus a control expression level for that corresponding analyte is associated with a decreased chance of response in that patient to the cellular therapeutic product versus a predetermined and/or historical likelihood of a response to the cellular therapeutic product. In certain embodiments, the decreased chance of response is measured with respect to a known and historical average likelihood of a response to the cellular therapeutic product in a population.

The term “predetermined” as used herein refers to an expected value or likelihood of an outcome based on information which does not include specific information relating to any particular patient who may be or may become the subject of cell therapy administration.

The terms “product” or “infusion product” are used interchangeably herein and refer to the T cell composition that is administered to the subject in need thereof. Typically, in CAR T-cell therapy, the T cell composition is administered as an infusion product.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement).

Furthermore, each type of T cells can be characterized with cell surface markers, as well known in the art. For instance, naïve T cells can be characterized as CCR7+, CD45RO−, and CD95−. Additional markers for naïve T cell include CD45RA+, CD62L+, CD27+, CD28+, CD127+, CD132+, CD25−, CD44−, and HLA-DR−. Surface markers to stem memory T cells (Tscm) include, without limitation, CD45RO−, CCR7+, CD45RA+, CD62L+(L-selectin), CD27+, CD28+, IL-7Ra+, CD95+, IL-2RP+, CXCR3+, and LFA−. Surface markers for effector memory T cells (Tem) include, without limitation, CCR7−, CD45RO+ and CD95+. Additional marker for effector memory T cells is IL-2Rβ+. For central memory T cells (Tcm), suitable markers include CD45RO+, CD95+, IL-2Rβ+, CCR7+ and CD62L+. For effector T cells (Teff), suitable markers include CD45RA+, CD95+, IL-2Rβ+, CCR7− and CD62L−, without limitation.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, International Publication No. WO 2008/081035, International Publication No. WO 2015/20096, International Publication No. WO 2016/191756, International Publication No. WO 2016/191755, International Publication No. WO 2019/079564, and International Publication No. WO 2021/092290, each of which are herein incorporated in their entireties. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy, as well as methods for making CAR T cells for cell therapy are disclosed in U.S. Patent Publication No. 2013/0287748, International Publication No. WO 2015/20096, International Publication No. WO 2016/191756, International Publication No. WO 2016/191755, International Publication No. WO 2019/079564, and International Publication No. WO 2021/092290, each of which are herein incorporated by reference in their entirety.

The term “engineered Autologous Cell Therapy,” or “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR). CAR positive (+) T cells are engineered to express an extracellular single chain variable fragment (scFv) with specificity for a particular tumor antigen linked to an intracellular signaling part comprising at least one costimulatory domain and at least one activating domain. The CAR scFv may be designed to target, for example, CD19, which is a transmembrane protein expressed by cells in the B cell lineage, including all normal B cells and B cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T cell ALL. Example CAR T cell therapies and constructs are described in U.S. Patent Publication Nos. 2013/0287748, 2014/0227237, 2014/0099309, and 2014/0050708, and these references are incorporated by reference in their entirety.

A “patient” or a “subject” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. In certain embodiments, a co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICOS, Ig alpha (CD79a), IL2R beta, IL2R gamma, IL7R alpha, integrin, ITGA4, ITGA6, ITGAD, ITGAE, ITGAL, ITGAM, ITGAX, ITGB2, ITGB7, ITGB1, KIRDS2, LAT, LFA-1, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMFI; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission. In some embodiments, the treatment may be prophylactic, in which case the treatment is administered before any symptoms of the condition are observed. The term “prophylaxis” as used herein means the prevention of or protective treatment for a disease or disease state. Prevention of a symptom, disease, or disease state may include reduction (e.g., mitigation) of one or more symptoms of the disease or disease state, e.g., relative to a reference level (e.g., the symptom(s) in a similar subject not administered the treatment). Prevention may also include delaying onset of one or more symptoms of the disease or disease state, e.g., relative to a reference level (e.g., the onset of the symptom(s) in a similar subject not administered the treatment). In embodiments, a disease is a disease described herein. In some embodiments, the disease is cancer. In some embodiments, the diseased state is CRS or neurotoxicity. In some embodiments, indicators of improvement or successful treatment include determination of the failure to manifest a relevant score on toxicity grading scale (e.g. CRS or neurotoxicity grading scale), such as a score of less than 3, or a change in grading or severity on the grading scale as discussed herein, such as a change from a score of 4 to a score of 3, or a change from a score of 4 to a score of 2, 1 or 0.

As used herein, “myeloid cells” are a subgroup of leukocytes that includes granulocytes, monocytes, macrophages, and dendritic cells.

In one embodiment, the terms “high” and “low” mean “above” and “below” the median value for a representative population of subjects. In one embodiment, the terms mean in the upper or lower quartiles, respectively. Both the mean and the quartile distribution may be determined by one of ordinary skill in the art by routine methods.

As used herein, the term “quartile” is a statistical term describing a division of observations into four defined intervals based upon the values of the data and how they compare to the entire set of observations.

As used herein, the term “Study day 0” is defined as the day the subject received the first CAR T cell infusion. The day prior to study day 0 will be study day −1. Any days after enrollment and prior to study day −1 will be sequential and negative integer-valued.

As used herein, the term “durable response” refers to the subjects who were in ongoing response at least by one year follow up post CAR T cell infusion. In one embodiment, “duration of response” is defined as the time from the first objective response to disease progression or to death due to disease relapse.

As used herein, the term “relapse” refers to the subjects who achieved a complete response (CR) or partial response (PR) and subsequently experienced disease progression.

As used herein, the term “non-response” refers to the subjects who had never experienced CR or PR post CAR T cell infusion, including subjects that with stable disease (SD) and progressive disease (PD).

As used herein, the term “objective response” refers to complete response (CR), partial response (PR), or non-response. It may be assessed per revised IWG Response Criteria for Malignant Lymphoma (Cheson et al., J Clin Oncol. 2007; 25(5):579-86).

As used herein, the term “complete response” refers to complete resolution of disease, which becomes not detectable by radio-imaging and clinical laboratory evaluation. No evidence of cancer at a given time.

As used herein, the term “partial response” refers to a reduction of greater than 30% of tumor without complete resolution.

As used herein “objective response rate” (ORR) is determine per International Working Group (IWG) 2007 criteria (Cheson et al. J Clin Oncol. 2007; 25(5):579-86).

As used herein “progression-free survival (PFS)” may be defined as the time from the T cell infusion date to the date of disease progression or death from any cause. Progression is defined per investigator's assessment of response as defined by IWG criteria (Cheson et al., J Clin Oncol. 2007; 25(5):579-86).

The term “overall survival (OS)” may be defined as the time from the T cell infusion date to the date of death from any cause.

As used herein, the expansion and persistence of CAR T cells in peripheral blood may be monitored by qPCR analysis, for example using CAR-specific primers for the scFv portion of the CAR (e.g., heavy chain of a CD19 binding domain) and its hinge/CD28 transmembrane domain. Alternatively, it may be measured by enumerating CAR cells/unit of blood volume.

As used herein, the scheduled blood draw for CAR T cells may be before CAR T cell infusion, Day 7, Week 2 (Day 14), Week 4 (Day 28), Month 3 (Day 90), Month 6 (Day 180), Month 12 (Day 360), and Month 24 (Day 720).

As used herein, the “peak of CAR T cell” is defined as the maximum absolute number of CAR+ PBMC/μL in serum attained after Day 0.

As used herein, the “time to Peak of CAR T cell” is defined as the number of days from Day 0 to the day when the peak of CAR T cell is attained.

As used herein, the “Area Under Curve (AUC) of level of CAR T cell from Day 0 to Day 28” is defined as the area under the curve in a plot of levels of CAR T cells against scheduled visits from Day 0 to Day 28. This AUC measures the total levels of CAR T cells overtime.

As used herein, the scheduled blood draw for cytokines is before or on the day of conditioning chemotherapy (Day −5), Day 0, Day 1, Day 3, Day 5, Day 7, every other day if any through hospitalization, Week 2 (Day 14), and Week 4 (Day 28).

As used herein, the “baseline” of cytokines is defined as the last value measured prior to conditioning chemotherapy.

As used herein, the fold change from baseline at Day X is defined as

$$\frac{Cytokine level at Day X - Baseline}{B aseline}$$

As used herein, the “peak of cytokine post baseline” is defined as the maximum level of cytokine in serum attained after baseline (Day −5) up to Day 28.

As used herein, the “time to peak of cytokine” post CAR T cell infusion is defined as the number of days from Day 0 to the day when the peak of cytokine was attained.

As used herein, the “Area Under Curve (AUC) of cytokine levels” from Day −5 to Day 28 is defined as the area under the curve in a plot of levels of cytokine against scheduled visits from Day −5 to Day 28. This AUC measures the total levels of cytokine overtime. Given the cytokine and CAR+ T cell are measured at certain discrete time points, the trapezoidal rule may be used to estimate the AUCs.

As used herein, treatment-emergent adverse events (TEAEs) are defined as adverse events (AE) with onset on or after the first dose of conditioning chemotherapy. Adverse events may be coded with the Medical Dictionary for Regulatory Activities (MedDRA) version 22.0 and graded using the National Cancer Institute (NCI) Common Terminology Criteria for Adverse Events (CTCAE) version 4.03. Cytokine Release Syndrome (CRS) events may be graded on the syndrome level per Lee and colleagues (Lee et al, 2014 Blood. 2014; 124(2):188-95. Individual CRS symptoms may be graded per CTCAE 4.03. Neurologic events may be identified with a search strategy based on known neurologic toxicities associated with CAR T immunotherapy, as described in, for example, Topp, M S et al. Lancet Oncology. 2015; 16(1):57-66.

Various aspects of the disclosure are described in further detail in the following subsections.

### Characterization of the Serum Protein Profile of Immunotherapy Cancer Patients

In some embodiments, the present disclosure provides methods to characterize the serum proteomic profile of a cancer patient prior to treatment with immunotherapy and/or pre-conditioning. In one embodiment, immunotherapy is selected from treatment with a chimeric receptor therapy (e.g., YESCARTA™ axicabtagene ciloleucel (axi-cel), TECARTUS™-brexucabtagene autoleucel/KTE-X19, KYMRIAH™ (tisagenlecleucel), etc), TCR, TIL, immune check point inhibitors, among others. In one embodiment, the immunotherapy product comprises autologous or allogeneic CAR T cells. In one embodiment, the immunotherapy comprises T-Cell Receptor-modified T cells. In one embodiment, the immunotherapy comprises tumor infiltrating lymphocytes (TILs). In one embodiment, the immunotherapy product comprises Induced Pluripotent Stem Cells (iPSCs). As described herein, in some embodiments, the serum protein characteristics are obtained through pre-specified protein sets and analyzed through OPI and machine learning models. In some embodiments, the serum levels may be measured by ELISA. In some embodiments, the serum protein profiles associate with adverse events of chimeric receptor therapy (e.g., axicabtagene ciloleucel (axi-cel)) and may be used to predict adverse events in response to all immunotherapies (e.g., T cells, non-T cells, TCR-based therapies, CAR-based therapies, bispecific T-cell engagers (BiTEs), and/or immune checkpoint blockade).

In one embodiment, the disclosure provides that baseline (pre-conditioning) serum levels of certain protein associated with metabolic processes and leukocyte activation correlate positively with, and can be biomarkers for, poor prognosis factors for immunotherapy including international prognostic index and baseline tumor burden. In one embodiment, the immunotherapy is T cell therapy. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells In one embodiment, the immunotherapy is CAR T cell or TCR T cell therapy. In one embodiment, the immunotherapy is anti-CD19 CAR T cell therapy.

Accordingly, in one embodiment, the disclosure provides a method of predicting international prognostic index and baseline tumor burden parameters in a cancer patient based on the baseline (pre-conditioning) serum levels of metabolic process markers and/or leukocyte activation markers in the patient.

In one embodiment, the disclosure provides that increased pre-treatment expression levels of at least one of CD19, MS4A1, TNFRSF17, BLK, FCRL2, FAM30A, PNOC, SPIB, and TCL1A are correlated with an increased likelihood of response to a cellular therapy product in a patient, while increased pre-treatment levels of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 are correlated with a reduced increased likelihood of response to a cellular therapy product in a patient. In one embodiment, this information is utilized to make decisions related to the immunotherapy including whether or not to administer immunotherapy, whether or not to administer immunotherapy as a second-line therapy or as a third-line therapy, what dosage of immunotherapy to administer, what dosage regimen to follow, and/or what agents should be administered to the patient prior to, after, and/or during immunotherapy administration.

In one embodiment, a high level of serum biomarkers is a level at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, or at least 100 fold over median. In one embodiment, the levels of the protein biomarkers are high or low, respectively, when they fall 0-0.1%, 0.1%-0.5%, 0.5%-1.0%, 1-5%, 5-10%, 10-15%, 15-20%, 20-25%, 25-30%, 30-35%, 35-40%, 40-45%, 45-50%, 50-55%, 55-60%, 60-65%, 65-70%, etc. 95%-100%) above or below, respectively, the median or the values specified above. All listed values may be modified by the term “above.”

In one embodiment, the disclosure provides a method of treating a subject with immunotherapy having a high tumor burden, wherein the immune activation mediated stress in the subject is reduced by administering one or more agents or treatments that result in a reduced inflammation (e.g., lower cytokine induction in the blood) and/or by using an alternative lymphodepleting regimen that does not comprise the administration of 500-600 mg/m²/day of cyclophosphamide and 30 mg/m²/day of fludarabine for 3 days prior to immunotherapy. In one embodiment, the subject has a high tumor burden (as assessed by SPD and/or tumor metabolic volume) when the baseline tumor burden (SPD) is greater than 2500, 3000, 3500, or 4000, preferably greater than 3000 mm² and/or the tumor metabolic volume is above the median for a representative tumor population (e.g., above 100, or above 150 ml).

In one embodiment, the disclosure provides a method of treating a subject with a high international prognostic index, wherein the immune activation mediated stress in the subject is reduced by administering one or more agents or treatments that result in a reduced inflammation (e.g., lower cytokine induction in the blood) and/or by using an alternative lymphodepleting regimen that does not comprise the administration of 500-600 mg/m²/day of cyclophosphamide and 30 mg/m²/day of fludarabine for 3 days prior to immunotherapy. In one embodiment, the subject has a high international prognostic index (IPI) when the IPI is greater than 1, 2 or 3.

In one embodiment, the immunotherapy is T cell therapy. In one embodiment, the T cell therapy is autologous. In one embodiment, the T cell therapy is allogeneic. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), iPSCs, checkpoint inhibitors, and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells. In one embodiment, the immunotherapy is CAR T cell or TCR T cell therapy. In one embodiment, the immunotherapy is anti-CD19 CAR T cell therapy. Examples of target tumor antigens are listed elsewhere in the specification. Examples of cancers that may be treated by the methods of the disclosure are also provided elsewhere in the specification.

In one embodiment, the agent(s) that is administered in combination with immunotherapy and reduces immune activation and/or endothelial cells disruption, wherein the combination therapy reduces cytokine induction and/or wherein the combination therapy reduces the endothelial cell disruption, is/are selected from anti-IL-1 (e.g. anakinra), T cell activation inhibitors (e.g., dasatinib), JAK inhibitors (e.g., filgotinib), anti-GM-CSF (e.g., lenzilumab), anti-TNF (e.g., infliximab), Ang2 inhibitors (e.g., azilsartan), anti-angiogenic therapies (e.g., bevacizumab), anti-IFNg (e.g., emapalumab-lzsg) etc. In one embodiment, the immunotherapy is administered in a combination therapy that enhances the proliferation of the T cells. In one embodiment, said combination therapy comprises treatment with pembrolizumab, lenalidomide, epcoritamab, and utoliumab. In one embodiment, said therapy comprises magrolimab (anti-CD47 antagonist), GSK3745417 (STING agonist), INCB001158 (ARG1/2 inhibitor), GS-1423 (CD73×TGFβ mAb), Selicrelumab (CD40 agonist), GS3583 (FLT3 agonist), Pexidartinib (CSF1R inhibitor, epacadostat (IDO1 inhibitor), GS9620 (TLR agonist). In one embodiment, the agent is selected from (i) a GM-CSF inhibitor selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule; (ii) a CSF1 inhibitor selected from RG7155, PD-0360324, MCS110/lacnotuzumab), or a biosimilar version of any one of the same; and a small molecule; and/or (iii) a GM-CSFR inhibitor and the CSF1R inhibitor selected from Mavrilimumab (formerly CAM-3001; MedImmune, Inc.); cabiralizumab (Five Prime Therapeutics); LY3022855 (IMC-CS4)(Eli Lilly), Emactuzumab, also known as RG7155 or RO5509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule. In some embodiments, additional treatments may be cytokines (e.g., IL-2, IL-15), stimulating antibodies (e.g., anti-41BB, OX-40), checkpoint blockade (e.g., CTLA4, PD-1), or innate immune stimulators (e.g., TLR, STING agonists). In some embodiments, additional treatments may be T cell-recruiting chemokines (e.g., CCL2, CCL1, CCL22, CCL17, and combinations thereof). In some embodiments, the additional therapy or therapies are administered systemically or intratumorally. In some embodiments, the additional therapy that is used in combination is administered together with conditioning and/or immunotherapy. In some embodiments, the additional therapy that is used in combination is administered sequentially with conditioning and/or immunotherapy.

In one embodiment, the agents may/should be administered to the patient prior to, after, and/or during immunotherapy administration to reduce Grade 3+ CRS in the subject. In one embodiment, the agent(s) is/are administered to the patient prior to CAR-T infusion, before the peak of CAR-T expansion (e.g., Day 0-6 post infusion), and/or at the peak CAR-T expansion (e.g., Day 7-14). In one embodiment, the peak of CAR-T expansion is Day 7-14 post infusion. In one embodiment, the peak of CAR-T expansion is Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, or Day 20 post-infusion. In one embodiment, the period after peak CAR-T expansion is the period between Day 14-28 post-infusion. In one embodiment, the period after peak CAR-T expansion is Day 1-Day 5, Day 5-Day 10, Day 10-Day 15, Day 15-Day 20, Day 20-Day 25; after Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, Day 25, Day 30, Day 35, Day 40, Day 45, Day 50, any day after peak expansion.

In one embodiment, the immunotherapy is combined with low dose radiation, promotion of T cell activity through immune checkpoint blockade, and/or T cell agonists. In one embodiment, the T cell agonist is selected from pembrolizumab, lenalidomide, epcoritamab, and utoliumab. In one embodiment, the combination agent is selected from check-point inhibitors (e.g., anti-PD1 antibodies, pembrolizumab (Keytruda), Cemiplimab (Libtayo), nivolumab (Opdivo); anti-PD-L1 antibodies, Atezolizumab (Tecentriq), Avelumab (Bavencio), Durvalumab (Imfinzi); and/or anti-CTLA-4 antibodies, Ipilimumab (Yervoy)).

In one embodiment, the pre-conditioning regimen is a lymphodepleting regimen. In one embodiment, the lymphodepletion therapy regimen(s) is/are selected from one of several possible regimens of cyclophosphamide/fludarabine, bendamustine, total body irradiation, Anti-CD45 (Apamistamab), and other chemotherapeutic agents (e.g., AVM0703, Busulfan, Thiotepa/Etoposide, Pentostatin). Additional conditioning methods and regimens can be found elsewhere in the specification.

In one embodiment, the disclosure provides a method of improving immunotherapy (e.g., CAR T cell treatment) by optimization of bridging therapy to modulate the tumor microenvironment to a more favorable immune permissive state. In one embodiment, the optimization comprises administering bridging therapy with Immunomodulatory imide drugs (IMIDs)/cereblon modulators (e.g., lenoalidomide, pomalidomide, iberdomide, and apremilast). In one embodiment, the optimization comprises administering bridging therapy with local radiation.

In one embodiment, the disclosure provides a method of improving immunotherapy (e.g. CAR T cell treatment) by optimization of bridging therapy to diminish tumor burden prior immunotherapy (e.g. CAR T cell treatment) administration. In one embodiment, the optimization comprises administering bridging therapy with R-CHOP, bendamustine, alkylating agents, and/or platinum-based agents. Other exemplary bridging therapies are described elsewhere in this application.

In one embodiment, the disclosure provides a method of improving immunotherapy (e.g., CAR T cell treatment) by optimization of conditioning treatment to modulate the tumor microenvironment to a more favorable immune permissive state (e.g., less myeloid inflammation in the TME). In one embodiment, the optimization comprises addition of local irradiation to cyclophosphamide/fludarabine conditioning. In one embodiment, the optimization comprises administration of platinum-based agents as conditioning agents.

In one embodiment, the disclosure provides a method of improving immunotherapy (e.g. CAR T cell treatment) by coadministration of biological response modifiers together or post-immunotherapy (e.g. CAR T cell treatment) administration to enable CAR T cell activity. In one embodiment, the method comprises administration of gamma chain cytokines (e.g., IL-2, IL-4, IL-7, IL-9, IL-15, and IL-21). In one embodiment, the method comprises administration of checkpoint blocking agents (e.g., anti-CTLA-4).

In one embodiment, the disclosure provides a method of improving immunotherapy (e.g., CAR T cell treatment) by reprogramming of T cells to overcome detrimental tumor microenvironments, including low T/M ratio, high tumor burden, high TME myeloid cell density and/or high TME myeloid inflammation levels. In one embodiment, the T cells are engineered to express gamma chain receptor cytokines. In one embodiment, the gamma chain receptor cytokines are expressed under constitutive or inducible promoters.

In one embodiment, the disclosure provides a method of improving CAR T cell treatment by optimizing T cell manufacturing to help CAR T cells overcome detrimental tumor microenvironments, wherein the characteristics of the tumor microenvironment that may be detrimental comprise low T/M ratio, high tumor burden, high TME myeloid cell density and/or high TME myeloid inflammation levels. In one embodiment, the characteristics of the TME that may be detrimental comprise low T/M ratio (within −0.5-4), high tumor burden (within 3000-40000 mm²), high myeloid cell density (within 1000-4000 cells/mm²) and/or high TME myeloid inflammation levels (within 27-2000). In one embodiment, the method comprises engineering CAR T cells to express gamma chain receptor cytokines. In one embodiment, the gamma chain receptor cytokines are expressed under constitutive or inducible promoters. In one embodiment, the method comprises growing the T cells in the presence of gamma chain cytokines such as IL-15.

### Clinical Outcomes

In some embodiments, the clinical outcome is complete response. In some embodiments, the clinical outcome is durable response. In some embodiments, the clinical outcome is complete response. In some embodiments, the clinical outcome is no response. In some embodiments, the clinical outcome is partial response. In some embodiments, the clinical outcome is objective response. In some embodiments, the clinical outcome is survival. In some embodiments, the clinical outcome is relapse.

In some embodiments, objective response (OR) is determined per the revised IWG Response Criteria for Malignant Lymphoma (Cheson, 2007) and determined by IWG Response Criteria for Malignant Lymphoma (Cheson et al. Journal of Clinical Oncology 32, no. 27 (September 2014) 3059-3067). Duration of Response is assessed. The Progression-Free Survival (PFS) by investigator assessment per Lugano Response Classification Criteria is evaluated.

In some embodiments, part of the clinical outcome is the evaluation of adverse events. In this regard, CRS grading was done according to Lee D W et al., (2014). Current concepts in the diagnosis and management of cytokine release syndrome. Blood. 2014 Jul. 10; 124(2): 188-195. Neurologic toxicity was assessed by monitoring patients for signs and symptoms of neurologic toxicities by ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the method comprises monitoring patients at least daily for 7 days at the certified healthcare facility following infusion for signs and symptoms of neurologic toxicities. In some embodiments, the method comprises monitoring patients for signs or symptoms of neurologic toxicities for 4 weeks after infusion.

In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

Clinical outcomes of CAR T cell treatment are dependent on the level of CAR T cells in the blood. In some embodiments, response, levels of CAR T cells in blood, or immune related factors is determined by follow up at about 1 day, about 2 days, about 3 days, about 4 days, about 5 days, about 6 days, or about 7 days after administration of engineered CAR T cells. In some embodiments, response, levels of CAR T cells in blood, or immune related factors is determined by follow up at about 1 week, about 2 weeks, about 3 weeks, or about 4 weeks after administration of engineered CAR T cells. In some embodiments, response, levels of CAR T cells in blood and/or immune related factors are determined by follow up at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administration of a engineered CAR T cells. In some embodiments, response, levels of CAR T cells in blood and/or immune related factors are determined by follow up at about 1 year, about 1.5 years, about 2 years, about 2.5 years, about 3 years, about 4 years, or about 5 years after administration of engineered CAR T cells.

In some embodiments, methods described herein may provide a clinical benefit to a subject. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, approximately 1%, 2%, 3%, 4%, 5%, 6%, 7%, 8%, 9%, 10%, 15%, 20%, 25%, 30%, 35%, 40%, 45%, 0%, 55%, 60%, 65%, 70%, 75%, 80%, 85%, 90% or 95% and any unenumerated % in between of patients achieve a clinical benefit. In some embodiments, the response rate is 1%, 2%, 3%, 4%, 5%, 6%, 7%, 8%, 9%, 9.5%, 10.5%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 25 55%, 56%, 57%, 58%, 59%, 60%, 61%, 62%, 63%, 64%, 65%, 66%, 67%, 68%, 69%, 70%, 71%. 72%, 73%, 74%, 75%, 76%, 77%, 78%, 79%, 80%, 81, 82, 83, 84, 85, 86, 87, 88, 89, 90, 91, 92, 93, 94, 95, 96, 97, 98, 99, or 100% or some other unenumerated percentage and range in between 1% and 100%. In some embodiments, the response rate is between 0%-10%, 10%-20%, 20%-30%, 30%-40%, 40%-50%, 50%-60%, 60%-70%, 70%-80%, 80%-90%, or 90%-100%. In some embodiments, the response rate is between 0%-1.%, 1%-1.5%, 1.5%-2%, 2%-3%, 3%-4%, 4%-5%, 5%-6%, 6%-7%, 7%-8%, 8%-9%, 9%-10%, 10%-15%, 15%-20%, 20-25%, 25%-30%, 35-40%, and so one and so forth, through 95%-100%.

### Chimeric Antigen Receptors

In one embodiment, the immunotherapy is CAR-T cell immunotherapy. Chimeric antigen receptors (CARs) are genetically engineered receptors. These engineered receptors may be inserted into and expressed by immune cells, including T cells and other lymphocytes in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016).

In some embodiments, a costimulatory domain which includes a truncated hinge domain (“THD”) further comprises some or all of a member of the immunoglobulin family such as IgG1, IgG2, IgG3, IgG4, IgA, IgD, IgE, IgM, or fragment thereof.

In some embodiments, the THD is derived from a human complete hinge domain (“CHD”). In other embodiments, the THD is derived from a rodent, murine, or primate (e.g., non-human primate) CHD of a costimulatory protein. In some embodiments, the THD is derived from a chimeric CHD of a costimulatory protein.

The costimulatory domain for the CAR of the disclosure may further comprise a transmembrane domain and/or an intracellular signaling domain. The transmembrane domain may be fused to the extracellular domain of the CAR. The costimulatory domain may similarly be fused to the intracellular domain of the CAR. In some embodiments, the transmembrane domain that naturally is associated with one of the domains in a CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this disclosure may be derived from (i.e., comprise) 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMFI; CD150; IPO-3), SLAMF4 (CD244; 2B4), SLAMF6 (NTB-A; Lyl08), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Optionally, short linkers may form linkages between any or some of the extracellular, transmembrane, and intracellular domains of the CAR. The linkers described herein, may also be used as a peptide tag. The linker peptide sequence may be of any appropriate length to connect one or more proteins of interest and is preferably designed to be sufficiently flexible so as to allow the proper folding and/or function and/or activity of one or both of the peptides it connects. Thus, the linker peptide may have a length of no more than 10, no more than 11, no more than 12, no more than 13, no more than 14, no more than 15, no more than 16, no more than 17, no more than 18, no more than 19, or no more than 20 amino acids. In some embodiments, the linker peptide comprises a length of at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the linker comprises at least 7 and no more than 20 amino acids, at least 7 and no more than 19 amino acids, at least 7 and no more than 18 amino acids, at least 7 and no more than 17 amino acids, at least 7 and no more than 16 amino acids, at least 7 and no more 15 amino acids, at least 7 and no more than 14 amino acids, at least 7 and no more than 13 amino acids, at least 7 and no more than 12 amino acids or at least 7 and no more than 11 amino acids. In certain embodiments, the linker comprises 15-17 amino acids, and in particular embodiments, comprises 16 amino acids. In some embodiments, the linker comprises 10-20 amino acids. In some embodiments, the linker comprises 14-19 amino acids. In some embodiments, the linker comprises 15-17 amino acids. In some embodiments, the linker comprises 15-16 amino acids. In some embodiments, the linker comprises 16 amino acids. In some embodiments, the linker comprises 3, 4, 5, 6, 7, 8, 9, 10, 11, 12, 13, 14, 15, 16, 17, 18, 19 or 20 amino acids.

In some embodiments, a spacer domain is used. In some embodiments, the spacer domain is derived from CD4, CD8a, CD8b, CD28, CD28T, 4-1BB, or other molecule described herein. In some embodiments, the spacer domains may include a chemically induced dimerizer to control expression upon addition of a small molecule. In some embodiments, a spacer is not used.

The intracellular (signaling) domain of the engineered T cells of the disclosure may provide signaling to an activating domain, which then activates at least one of the normal effector functions of the immune cell. Effector function of a T cell, for example, may be cytolytic activity or helper activity including the secretion of cytokines.

In certain embodiments, suitable intracellular signaling domain include (i.e., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMFI; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

### Antigen Binding Molecules

Suitable CARs and TCRs may bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (“scFv”). A scFv is a single chain antibody fragment having the variable regions of the heavy and light chains of an antibody linked together. See U.S. Pat. Nos. 7,741,465 and 6,319,494, as well as Eshhar et al., Cancer Immunol Immunotherapy (1997) 45: 131-136. A scFv retains the parent antibody's ability to interact specifically with target antigen. scFv's are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR or TCR such that it is capable of recognizing and binding to the antigen of interest. Bispecific and multispecific CARs and TCRs are contemplated within the scope of the disclosure, with specificity to more than one target of interest.

In some embodiments, the polynucleotide encodes a CAR or TCR comprising a (truncated) hinge domain and an antigen binding molecule that specifically binds to a target antigen. In some embodiments, the target antigen is a tumor antigen. In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface antigens.

### Engineered T Cells and Products

In one embodiment, the immunotherapy is T cell therapy. In some embodiments, the donor T cells for use in the T cell therapy are obtained from the patient (e.g., for an autologous T cell therapy). In other embodiments, the donor T cells for use in the T cell therapy are obtained from a subject that is not the patient. In certain embodiments, the T cell is a tumor-infiltrating lymphocyte (TIL), engineered autologous T cell (eACT™), an allogeneic T cell, a heterologous T cell, or any combination thereof. In some embodiments, the T cells are obtained from a donor subject. In some embodiments, the donor subject is human patient afflicted with a cancer or a tumor. In some embodiments, the donor subject is a human patient not afflicted with a cancer or a tumor.

In one embodiment, the cells are obtained from a subject. In one embodiment, the cells are Induced Pluripotent Stem Cells (iPSCs). T cells may be obtained from, e.g., peripheral blood mononuclear cells, bone marrow, lymph node tissue, cord blood, thymus tissue, tissue from a site of infection, ascites, pleural effusion, spleen tissue, tumors, or differentiated in vitro. In addition, the T cells may be derived from one or more T cell lines available in the art. T cells may also be obtained from a unit of blood collected from a subject using any number of techniques known to the skilled artisan, such as FICOLL™ separation and/or apheresis. In some embodiments, the cells collected by apheresis are washed to remove the plasma fraction and placed in an appropriate buffer or media for subsequent processing. In some embodiments, the cells are washed with PBS. As will be appreciated, a washing step may be used, such as by using a semi-automated flow through centrifuge, e.g., the Cobe™ 2991 cell processor, the Baxter CytoMate™, or the like. In some embodiments, the washed cells are resuspended in one or more biocompatible buffers, or other saline solution with or without buffer. In some embodiments, the undesired components of the apheresis sample are removed. Additional methods of isolating T cells for a T cell therapy are disclosed in U.S. Patent Pub. No. 2013/0287748, which is herein incorporated by references in its entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+, CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8+ cells are further sorted into naive, central memory, and effector cells by identifying cell surface antigens that are associated with each of these types of CD8+ cells. In some embodiments, the expression of phenotypic markers of central memory T cells includes expression of CCR7, CD3, CD28, CD45RO, CD62L, and CD127 and negative for granzyme B. In some embodiments, central memory T cells are CD8+, CD45RO+, and CD62L+T cells. In some embodiments, effector T cells are negative for CCR7, CD28, CD62L, and CD127 and positive for granzyme B and perforin. In some embodiments, CD4+ T cells are further sorted into subpopulations. For example, CD4+ T helper cells may be sorted into naive, central memory, and effector cells by identifying cell populations that have cell surface antigens.

In some embodiments, the immune cells, e.g., T cells, are genetically modified (engineered) following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, by way of non-limiting example, in U.S. Pat. Nos. 6,905,874, 6,867,041, and 6,797,514, and in International Publication Nos. WO 2015/20096, WO 2016/191756, WO 2016/191755, WO 2019/079564, and WO 2021/092290, each of which are herein incorporated by reference in their entirety. The contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, a composition comprising engineered T cells comprises a pharmaceutically acceptable carrier, diluent, solubilizer, emulsifier, preservative and/or adjuvant. In some embodiments, the composition comprises an excipient.

In some embodiments, the composition is selected for parenteral delivery, for inhalation, or for delivery through the digestive tract, such as orally. The preparation of such pharmaceutically acceptable compositions is within the ability of one skilled in the art. In some embodiments, buffers are used to maintain the composition at physiological pH or at a slightly lower pH, typically within a pH range of from about 5 to about 8. In some embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In some embodiments, the vehicle for parenteral injection is sterile distilled water in which composition described herein, with or without at least one additional therapeutic agent, is formulated as a sterile, isotonic solution, properly preserved. In some embodiments, the preparation involves the formulation of the desired molecule with polymeric compounds (such as polylactic acid or polyglycolic acid), beads or liposomes, that provide for the controlled or sustained release of the product, which are then be delivered via a depot injection. In some embodiments, implantable drug delivery devices are used to introduce the desired molecule.

In some embodiments, the engineered T cells are administered at a therapeutically effective amount. For example, a therapeutically effective amount of the engineered T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the therapeutically effective amount of the engineered viable T cells is between about 1×10⁶ and about 2×10⁶ engineered viable T cells per kg body weight up to a maximum dose of about 1×10⁸ engineered viable T cells.

In some embodiments, the engineered T cells are anti-CD19 CART T cells. In some embodiments, the anti-CD19 CAR T cells are the axicabtagene ciloleucel product, YESCARTA™ axicabtagene ciloleucel (axi-cel), TECARTUS™-brexucabtagene autoleucel/KTE-X19, KYMRIAH™ (tisagenlecleucel), lisocabtagene maraleucel, In some embodiments, the engineered T cells are anti-BCMA CAR T cells, such as Idecabtagene vicleucel/bb2121, etc. In some embodiments, the product meets commercial specifications. In some embodiments, the product does not meet commercial specifications (out-of-specification product, OOS). In some embodiments, the OOS product comprises fewer, less differentiated CCR7+ T_{N} and T_{CM} and a greater proportion of more differentiated CCR7− T_{EM}+T_{EFF} cells than the axicabtagene ciloleucel product that meets commercial specifications. In some embodiments, the OOS product results in a median peak CAR T cell level after administration that is lower than that of the commercial product. In some embodiments, the OOS product still showed a manageable safety profile and meaningful clinical benefit.

The application also provides dosages and administrations of cells prepared by the methods of the application, for example, an infusion bag of CD19-directed genetically modified autologous T cell immunotherapy, comprises a suspension of chimeric antigen receptor (CAR)-positive T cells in approximately 68 mL for infusion. In some embodiments, the CAR T cells are formulated in approximately 40 mL for infusion. In some embodiments, the CAR T cell product is formulated in a total volume of 35, 40, 45, 50, 55, 60, 65, 70, 75, 80, 85, 90, 95, 100, 200, 300, 400, 500, 500, 700, 800, 900, 1000 mL. In one aspect, the dosage and administration of cells prepared by the methods of the application, for example, an infusion bag of CD19-directed genetically modified autologous T cell immunotherapy, comprises a suspension of 1×10⁶ CAR-T positive cells in approximately 40 mL. The target dose may be between about 1×10⁶ and about 2×10⁶ CAR-positive viable T cells per kg body weight, with a maximum of 2×10⁸ CAR-positive viable T cells.

In some embodiments, the dosage form comprises a cell suspension for infusion in a single-use, patient-specific infusion bag; the route of administration is intravenous; the entire contents of each single-use, patient-specific bag is infused by gravity or a peristaltic pump over 30 minutes. In one embodiment, the dosing regimen is a single infusion consisting of 2.0×10⁶ anti-CD19 CAR T cells/kg of body weight (±20%), with a maximum dose of 2×10⁸ anti-CD19 CAR T cells (for subjects ≥100 kg). In some embodiments, the T cells that make up the dose are CD19 CAR-T cells.

### Conditioning Agents

In some embodiments, the subject is administered a conditioning agent prior to immunotherapy. In some embodiments, conditioning is done with radiation treatment. In some embodiments, the conditioning therapy is a lymphodepleting chemotherapy.

In one embodiment, the conditioning therapy comprises an alkylating agent selected from the group consisting of melphalan, chlorambucil, cyclophosphamide, mechlorethamine, mustine (HN2), uramustine, uracil mustard, melphalan, chlorambucil, ifosfamide, bendamustine, carmustine, lomustine, streptozocin, alkyl sulfonates, busulfan, thiotepa or its analogues, and any combination thereof; a purine analogs selected from the group consisting of azathioprine, 6-mercaptopurine, mercaptopurine, thiopurines, thioguanine, fludarabine, pentostatin, cladribine, and any combination thereof; and/or a platinum-based preconditioning agents selected from the group consisting of platinum, cisplatin, carboplatin, nedaplatin, oxaliplatin, satraplatin, triplatin tetranitrate, procarbazine, altretamine, triazenes, dacarbazine, mitozolomide, temozolomide, dacarbazine, temozolomide, and any combination thereof.

In another embodiment, the one or more preconditioning agents can include platinum-based chemotherapeutic agents. In certain embodiments, the platinum-based chemotherapeutic agents are selected from the group consisting of platinum, cisplatin, carboplatin, nedaplatin, oxaliplatin, satraplatin, triplatin tetranitrate, procarbazine, altretamine, triazenes, dacarbazine, mitozolomide, temozolomide, dacarbazine, temozolomide, any analogues or functional derivatives thereof, and any combination thereof.

In another embodiment, the one or more preconditioning agents can include purine analogues. In certain embodiments, the purine analogues are selected from the group consisting of azathioprine, 6-mercaptopurine, mercaptopurine, thiopurines, thioguanine, fludarabine, pentostatin, cladribine, any analogue or functional derivative thereof, and any combination thereof. In one embodiment, the one or more preconditioning agents includes fludarabine.

In some embodiments, the one or more preconditioning agents can include cyclophosphamide and a purine analog. The purine analogues can be selected from the group consisting of azathioprine, 6-mercaptopurine, mercaptopurine, thiopurines, thioguanine, fludarabine, pentostatin, cladribine, any analogue or functional derivative thereof, and any combination thereof. In one particular embodiment, the one or more preconditioning agents include cyclophosphamide and pentostatin. In one particular embodiment, the one or more preconditioning agents include cyclophosphamide and fludarabine. By way of non-limiting example, dosing amounts and regimens of cyclophosphamide and fludarabine are described in at least International Publication No. WO 2019/079564, International Publication No. WO 2021/092290, International Publication No. WO 2015/20096, and International Publication No. WO 2016/191755, each of which are herein incorporated by reference in their entirety.

In certain embodiments, a first dose (also applies to repeated doses) of the one or more preconditioning agents is administered to the patient. For example, in some embodiments, a first dose of cyclophosphamide is about 300 mg/m²/day to about 2000 mg/m²/day. In another embodiment, the first dose of cyclophosphamide is higher than 300 mg/m²/day and lower than 2000 mg/m²/day. In other embodiments, the dose of cyclophosphamide is about 350 mg/m²/day-about 2000 mg/m²/day, at least about 400 mg/m²/day-about 2000 mg/m²/day, about 450 mg/m²/day-about 2000 mg/m²/day, about 500 mg/m²/day-about 2000 mg/m²/day, about 550 mg/m²/day-about 2000 mg/m²/day, or about 600 mg/m²/day-about 2000 mg/m²/day. In other embodiments, the dose of cyclophosphamide is about 350 mg/m²/day-about 1500 mg/m²/day, about 350 mg/m²/day-about 1000 mg/m²/day, about 400 mg/m²/day-about 900 mg/m²/day, about 450 mg/m²/day-about 800 mg/m²/day, about 450 mg/m²/day-about 700 mg/m²/day, about 500 mg/m²/day-about 600 mg/m²/day, or about 300 mg/m²/day-about 500 mg/m²/day. In another embodiment, the dose of cyclophosphamide is about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day.

In other embodiments, the first dose (also applies to repeated doses) of cyclophosphamide is about 200 mg/m²/day to about 3000 mg/m²/day. In another embodiment, the first dose of cyclophosphamide is higher than 200 mg/m²/day and lower than 3000 mg/m²/day. In other embodiments, the dose of cyclophosphamide is about 200 mg/m²/day-about 3000 mg/m²/day, about 300 mg/m²/day-about 3000 mg/m²/day, about 400 mg/m²/day-about 3000 mg/m²/day, about 500 mg/m²/day-about 3000 mg/m²/day, about 600 mg/m²/day-about 3000 mg/m²/day, about 700 mg/m²/day-about 3000 mg/m²/day, about 800 mg/m²/day-about 3000 mg/m²/day, about 900 mg/m²/day-about 3000 mg/m²/day, about 1000 mg/m²/day-about 3000 mg/m²/day, about 1100 mg/m²/day-about 3000 mg/m²/day, about 1200 mg/m²/day-about 3000 mg/m²/day, about 1300 mg/m²/day-about 3000 mg/m²/day, about 1400 mg/m²/day-about 3000 mg/m²/day, about 1500 mg/m²/day-about 3000 mg/m²/day, about 1600 mg/m²/day-about 3000 mg/m²/day, about 1700 mg/m²/day-about 3000 mg/m²/day, about 1800 mg/m²/day-about 3000 mg/m²/day, about 1900 mg/m²/day-about 3000 mg/m²/day, about 2000 mg/m²/day-about 3000 mg/m²/day, about 200 mg/m²/day-about 2900 mg/m²/day, about 400 mg/m²/day-about 2800 mg/m²/day, about 500 mg/m²/day-about 2700 mg/m²/day, about 600 mg/m²/day-about 2600 mg/m²/day, about 700 mg/m²/day-about 2500 mg/m²/day, about 800 mg/m²/day-about 2400 mg/m²/day, about 900 mg/m²/day-about 2350 mg/m²/day, about 1000 mg/m²/day-about 2300 mg/m²/day, about 1100 mg/m²/day-about 2250 mg/m²/day, or about 1110 mg/m²/day-about 2220 mg/m²/day. In one embodiment, the first dose of cyclophosphamide is 200 mg/m²/day. In another embodiment, the first dose of cyclophosphamide is 300 mg/m²/day. In another embodiment, the first dose of cyclophosphamide is 500 mg/m²/day.

In some embodiments, a first dose (also applies to repeated doses) of fludarabine is about 20 mg/m²/day to about 900 mg/m²/day. In some embodiments, a dose of fludarabine is higher than 30 mg/m²/day and lower than 900 mg/m²/day. In some embodiments, a dose fludarabine is about 35 mg/m²/day-about 900 mg/m²/day, about 40 mg/m²/day-about 900 mg/m²/day, about 45 mg/m²/day-about 900 mg/m²/day, about 50 mg/m²/day-about 900 mg/m²/day, about 55 mg/m²/day-about 900 mg/m²/day, or about 60 mg/m²/day-about 900 mg/m²/day. In some embodiments, a dose of fludarabine is about 35 mg/m²/day-about 900 mg/m²/day, about 35 mg/m²/day-about 800 mg/m²/day, about 35 mg/m²/day-about 700 mg/m²/day, about 35 mg/m²/day-about 600 mg/m²/day, about 35 mg/m²/day-about 500 mg/m²/day, about 35 mg/m²/day-about 400 mg/m²/day, about 35 mg/m²/day-about 300 mg/m²/day, about 35 mg/m²/day-about 200 mg/m²/day, about 35 mg/m²/day-about 100 mg/m²/day, about 40 mg/m²/day-about 90 mg/m²/day, about 45 mg/m²/day-about 80 mg/m²/day, about 45 mg/m²/day-about 70 mg/m²/day, or about 50 mg/m²/day-about 60 mg/m²/day. In some embodiments, a dose of fludarabine is about 20 mg/m²/day, about 25 mg/m²/day, about 30 mg/m²/day, about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, about 100 mg/m²/day, about 200 mg/m²/day, or about 300 mg/m²/day. In some embodiments, a dose of fludarabine is about 20 mg/m²/day, about 25 mg/m²/day, about 30 mg/m²/day, about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In other embodiments, the dose of fludarabine is about 110 mg/m²/day, 120 mg/m²/day, 130 mg/m²/day, 140 mg/m²/day, 150 mg/m²/day, 160 mg/m²/day, 170 mg/m²/day, 180 mg/m²/day, or 190 mg/m²/day. In some embodiments, the dose of fludarabine is about 210 mg/m²/day, 220 mg/m²/day, 230 mg/m²/day, 240 mg/m²/day, 250 mg/m²/day, 260 mg/m²/day, 270 mg/m²/day, 280 mg/m²/day, or 290 mg/m²/day. In one particular embodiment, the dose of fludarabine is about 20 mg/m²/day. In one particular embodiment, the dose of fludarabine is about 25 mg/m²/day. In another embodiment, dose of fludarabine is about 30 mg/m²/day. In another embodiment, dose of fludarabine is about 60 mg/m²/day.

The timing of the administration of the one or more preconditioning agents can be adjusted to maximize effect. In certain embodiments, the one or more preconditioning agents comprise at two or more preconditioning agents. The two or more preconditioning agents can be administered concurrently or sequentially. In one particular embodiment, a first preconditioning agent, e.g., cyclophosphamide, is administered to the patient prior to or after a second preconditioning agent, e.g., fludarabine.

The doses of cyclophosphamide and fludarabine can be raised or lowered together or independently. For example, the dose of cyclophosphamide can be increased while the dose of fludarabine is decreased, and the dose of cyclophosphamide can be decreased while the dose of fludarabine is increased. Alternatively, the dose of both cyclophosphamide and fludarabine can be increased or decreased together. In some embodiments, the dose of cyclophosphamide is 300 mg/m²/day and the dose of fludarabine is 20 mg/m²/day. In other embodiments, the dose of cyclophosphamide is 300 mg/m²/day and the dose of fludarabine is 30 mg/m²/day. In other embodiments, the dose of cyclophosphamide is 300 mg/m²/day and the dose of fludarabine is 60 mg/m²/day. In other embodiments, the dose of cyclophosphamide is 500 mg/m²/day and the dose of fludarabine is 20 mg/m²/day. In other embodiments, the dose of cyclophosphamide is 500 mg/m²/day and the dose of fludarabine is 30 mg/m²/day. In other embodiments, the dose of cyclophosphamide is 500 mg/m²/day and the dose of fludarabine is 60 mg/m²/day. In other embodiments, the dose of cyclophosphamide is 200 mg/m²/day and the dose of fludarabine is 20 mg/m²/day. In other embodiments, the dose of cyclophosphamide is 200 mg/m²/day and the dose of fludarabine is 30 mg/m²/day. In other embodiments, the dose of cyclophosphamide is 200 mg/m²/day and the dose of fludarabine is 60 mg/m²/day.

As described herein, the day that a T cell therapy is administered is designated as day 0. The one or more preconditioning agents can be administered at any time prior to administration of the T cell therapy. In some embodiments, the administration of the one or more preconditioning agents begins at least seven days, at least six days, at least five days, at least four days, at least three days, at least two days, or at least one day prior to the administration of the T cell therapy. In other embodiments, the administration of the one or more preconditioning agents begins at least eight days, at least nine days, at least ten days, at least eleven days, at least twelve days, at least thirteen days, or at least fourteen days prior to the administration of the T cell therapy. In one embodiment, the administration of the one or more preconditioning agents begins about seven days prior to the administration of the T cell therapy. In another embodiment, the administration of the one or more preconditioning agents begins about five days prior to the administration of the T cell therapy.

In one embodiment, the administration of a first preconditioning agent begins about seven days prior to the administration of the T cell therapy, and the administration of a second preconditioning agent begins about five days prior to administration of the T cell therapy. In one particular embodiment, a first preconditioning agent is administered to the patient for two days at about seven days and about six days prior to the administration of the T cell therapy. In another embodiment, a second preconditioning agent is administered to the patient for five days at about five, four, three, two, and one day prior to the administration of the T cell therapy. In another embodiment, a first preconditioning agent is administered to the patient for three days at about five, four, and three days prior to the administration of the T cell therapy.

In one particular embodiment, administration of the cyclophosphamide begins about seven days prior to the administration of the T cell therapy, and the administration of a purine analog (e.g., fludarabine or pentostatin) begins about five days prior to the administration of the T cell therapy. In another embodiment, administration of the cyclophosphamide begins about five days prior to the administration of the T cell therapy, and the administration of a purine analog (e.g., fludarabine or pentostatin) begins about five days prior to the administration of the T cell therapy.

The timing of the administration of each component can be adjusted to maximize effect. In general, the one or more preconditioning agents can be administered daily. In some embodiments, the one or more preconditioning agents are administered daily for about two days, for about three days, for about four days, for about five days, for about six days, or for about seven days. In some embodiments, the one or more preconditioning agents can be administered daily for at least one day, at least two days, at least three days, at least four days, at least five days, at least six days, or at least seven days. In one particular embodiment, the one or more preconditioning agents are administered daily for about three days.

As described herein, the day the T cell therapy is administered to the patient is designated as day 0. In some embodiments, the one or more preconditioning agents, e.g., the cyclophosphamide, is administered to the patient on day 7 and day 6 prior to day 0 (i.e., day −7 and day −6). In other embodiments, the one or more preconditioning agents, e.g., the cyclophosphamide, is administered to the patient on day −5, day −4, and day −3. In some embodiments, the one or more preconditioning agents, e.g., the fludarabine, is administered to the patient on day −5, day −4, day −3, day −2, and day −1. In other embodiments, the one or more preconditioning agents, e.g., fludarabine, is administered to the patient on day −5, day −4, and day −3.

The one or more preconditioning agents, e.g., the cyclophosphamide and fludarabine, can be administered on the same or different days. If cyclophosphamide and fludarabine are administered on the same day, the cyclophosphamide dose can be administered either before or after the fludarabine dose. In one embodiment, the cyclophosphamide dose is administered to the patient on day −7 and day −6, and the fludarabine dose is administered to the patient on day −5, day −4, day −3, day −2, and day −1. In another embodiment, the cyclophosphamide dose is administered to the patient on day −5, day −4, and day −3, and the fludarabine dose is administered to the patient on day −5, day −4, and day −3.

In certain embodiments, the one or more preconditioning agents, e.g., cyclophosphamide and fludarabine, can be administered concurrently or sequentially. In one embodiment, cyclophosphamide is administered to the patient prior to fludarabine. In another embodiment, cyclophosphamide is administered to the patient after fludarabine.

Routes and regimes for administrating the one or more preconditioning agents are known in the art, and are described, for example, at least in International Publication No. WO 2019/079564, International Publication No. WO 2021/092290, International Publication No. WO 2015/20096, and International Publication No. WO 2016/191755 each of which are herein incorporated by reference in their entirety.

### Cancers

The methods disclosed herein may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In other embodiments, the methods induce a partial response.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In some embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, Waldenstrom macroglobulinemia, DLBCL arising from FL, high grade B cell lymphoma, or a combination thereof.

In some embodiments, the cancer is a myeloma. In some embodiments, the cancer is multiple myeloma. In some embodiments, the cancer is leukemia. In some embodiments, the cancer is acute myeloid leukemia. In some embodiments, the cancer is relapsed or refractory large B-cell lymphoma (possibly, after two or more lines of systemic therapy), including diffuse large B-cell lymphoma (DLBCL) not otherwise specified, primary mediastinal large B-cell lymphoma, high grade B-cell lymphoma, and DLBCL arising from follicular lymphoma, or relapsed or refractory follicular lymphoma (FL) (possibly, after two or more lines of systemic therapy), or relapsed or refractory mantle cell lymphoma (MCL).

In some embodiments, the cancer is Non-Hodgkin lymphoma. In some embodiments, the cancer is relapsed/refractory NHL. In some embodiments, the cancer is mantle cell lymphoma.

In some embodiments, the cancer is advanced-stage indolent non-Hodgkin lymphoma (iNHL), including follicular lymphoma (FL) and marginal zone lymphoma (MZL). In some embodiments, the patient has had relapsed/refractory disease after ≥2 prior lines of therapy, including an anti-CD20 monoclonal antibody with an alkylating agent. In some embodiments, the patient may have received a PI3K inhibitor. In some embodiments, the patient may (also) have received autologous stem cell transplantation. In some embodiments, the patient undergoes leukapheresis to obtain T cells for CAR T cell manufacturing, followed by conditioning chemotherapy with cyclophosphamide at 500 mg/m²/day and fludarabine at 30 mg/m²/day administered on days −5, −4, and −3; on day 0, the patient may receive a single intravenous infusion of CAR T cell therapy (e.g., axicabtagene ciloleucel, brexucabtagene autoleucel) at a target dose of 2×10⁶ CAR T cells/kg. In some embodiments, additional infusions may be given at a later period. In some embodiments, if the patient progresses after responding at the month 3 assessment after initial administration, the patient may receive retreatment with CAR T cell treatment (e.g., axicabtagene ciloleucel, brexucabtagene autoleucel). In some embodiments, the patient may receive bridging therapy. Examples of bridging therapies are provided elsewhere in the specification, including the Examples. In some embodiments, the patient experiences CRS. In some embodiments, CRS is managed using any one of the protocols described in this application, including the Examples. In some embodiments, CRS is managed with tocilizumab, corticosteroids and/or vasopressor.

In some embodiments, the cancer is relapsed/refractory indolent Non-Hodgkin Lymphoma and the method of treating a subject in need thereof comprises administering to the subject a therapeutically effective amount of CAR T cells as a retreatment, wherein the subject has previously received a first treatment with CAR T cells. In some embodiments, the first treatment with CAR T cells may have been administered as a first line therapy or a second line therapy, optionally wherein the lymphoma is R/R follicular lymphoma (FL) or marginal zone lymphoma (MZL) and optionally wherein the previous prior lines of therapy included anti-CD20 monoclonal antibody combined with an alkylating agent. In some embodiments, the conditioning therapy comprises fludarabine 30 mg/m² IV and cyclophosphamide 500 mg/m² IV on Days −5, −4, and −3. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cells are axicabtagene ciloleucel CAR T cells. In some embodiments, the retreatment eligibility criteria include response of a CR or PR at the month 3 disease assessment with subsequent progression; no evidence of CD19 loss in progression biopsy by local review; and/or no Grade 4 CRS or neurologic events, or life-threatening toxicities with the first treatment with CAR T cells. In some embodiments, the method of treatment is that followed by the clinical trial (NCT03105336).

In some embodiments, the cancer is NHL and the immunotherapy (e.g., CAR T or TCR T cell treatment) is administered as a first line therapy. In some embodiments, the cancer is LBCL. In some embodiments, the LBCL is high risk/high grade LBCL with MYC and BCL2 and/or BCL6 translocations or DLBCL with IPI score≥3 any time before enrollment. In some embodiments, the first line therapy comprises CAR T cell treatment in combination with an anti-CD20 monoclonal antibody and anthracycline-containing regimen. In some embodiments, the CAR T cell treatment is administered first. In some embodiments, the anti-CD20 monoclonal antibody/anthracycline-containing regimen is administered first. In some embodiments, the treatments are administered at least 2 weeks, at least 4 weeks, at least 6 weeks, at least 1 month, at least 2 months, at least 3 months, at least 4 months, at least 5 months, less than a year apart, etc. In some embodiments, the method further comprises bridging therapy administered after leukapheresis and completed prior to initiating conditioning chemotherapy. In some embodiments, additional inclusion criteria include age ≥18 years and ECOG PS 0-1. In some embodiments, the conditioning therapy comprises fludarabine 30 mg/m² IV and cyclophosphamide 500 mg/m² IV on Days −5, −4, and −3. Other exemplary beneficial preconditioning treatment regimens are described in U.S. Provisional Patent Applications 62/262,143 and 62/167,750 and U.S. Pat. Nos. 9,855,298 and 10,322,146, which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient. Another embodiment comprises serum cyclophosphamide and fludarabine at days −4, −3, and −2 prior to T cell administration at a dose of 500 mg/m² of body surface area of cyclophosphamide per day and a dose of 30 mg/m² of body surface area per day of fludarabine during that period of time. Another embodiment comprises cyclophosphamide at day −2 and fludarabine at day's −4, −3, and −2 prior to T cell administration, at a dose of 900 mg/m² of body surface area of cyclophosphamide and a dose of 25 mg/m² of body surface area per day of fludarabine during that period of time. In another embodiment, the conditioning comprises cyclophosphamide and fludarabine at days −5, −4 and −3 prior to T cell administration at a dose of 500 mg/m² of body surface area of cyclophosphamide per day and a dose of 30 mg/m² of body surface area of fludarabine per day during that period of time. Other preconditioning embodiments comprise 200-300 mg/m² of body surface area of cyclophosphamide per day and a dose of 20-50 mg/m² of body surface area per day of fludarabine for three days. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cell treatment comprises anti-CD19 CAR T cells. In some embodiments, the CAR T cell treatment comprises axicabtagene ciloleucel or YESCARTA™ In some embodiments, the CAR T cell treatment comprises TECARTUS™-brexucabtagene autoleucel or KYMRIAH™ (tisagenlecleucel), etc), Idecabtagene vicleucel/bb2121.

In another embodiment, the disclosure provides a method of treating cancer in a subject in need thereof, comprising administering a therapeutically effective amount of CD19 CAR-T treatment to a subject in which the number of lines of prior therapy are 1-2; 3; 4; or ≥5. In one embodiment, the disclosure provides a method of treating cancer in a subject in need thereof, comprising administering a therapeutically effective amount of CD19 CAR-T treatment to a subject in which the number of lines of prior therapy are 1-2. The cancer may be any one of the above listed cancers. The CD19 CAR-T treatment may be any one of the above listed CD19 CAR-T treatments. In some embodiments, the CD19 CAR-T treatment is used as first line of treatment. In some embodiments, the CD19 CAR-T treatment is used as a second line of treatment.

In one embodiment, the CD19 CAR-T treatment is any of the of CD19 CAR-T treatments described above. In one embodiment, the CD19 CAR-T treatment comprises axicabtagene ciloleucel treatment. In embodiments, the cancer is refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In one embodiment, a subject selected for axicabtagene ciloleucel treatment has refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In some embodiments, axicabtagene ciloleucel treatment is used as a second line of treatment, where the first line therapy is CHOP, i.e., Cyclophosphamide (Cytoxan®), Doxorubicin (hydroxydoxorubicin), Vincristine (Oncovin®), and Prednisone. In some embodiments, axicabtagene ciloleucel treatment is used as a second line of treatment, where the first line therapy is R-CHOP (CHOP plus Rituximab).

In embodiments, a patient is selected for second-line axicabtagene ciloleucel treatment that has relapsed or refractory disease after first-line chemoimmunotherapy, refractory disease defined as no complete remission to first-line therapy; individuals who are intolerant to first-line therapy are excluded. progressive disease (PD) as best response to first-line therapy, stable disease (SD) as best response after at least 4 cycles of first-line therapy (eg, 4 cycles of R-CHOP), partial response (PR) as best response after at least 6 cycles and biopsy-proven residual disease or disease progression ≤12 months of therapy, and/or relapsed disease defined as complete remission to first-line therapy followed by biopsy-proven relapse ≤12 months of first-line therapy. In some embodiments, a patient selected for second-line axicabtagene ciloleucel treatment is provided conditioning therapy comprising fludarabine 30 mg/m² IV and cyclophosphamide 500 mg/m² IV on Days −5, −4, and −3. In some embodiments, axicabtagene ciloleucel treatment is used as a second line of treatment.

### Combination Treatments

Compositions comprising CAR-expressing immune effector cells disclosed herein may be administered in conjunction (before, after, and/or concurrently with T cell administration) with any number of chemotherapeutic agents. In some embodiments, the antigen binding molecule, transduced (or otherwise engineered) cells (such as CARs), and the chemotherapeutic agent are administered each in an amount effective to treat the disease or condition in the subject. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylol melamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; Polysaccharide K (PSK); razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising CAR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone, R-CHOP (CHOP plus Rituximab), and G-CHOP (CHOP plus obinutuzumab).

In some embodiments, the chemotherapeutic agent is administered at the same time or within one week after the administration of the engineered cell. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein (before, after, and/or concurrently with T cell administration). For example, potentially useful additional therapeutic agents include PD-1 inhibitors such as nivolumab (OPDIVO®), pembrolizumab (KEYTRUDA®), Cemiplimab (Libtayo), pidilizumab (CureTech), and atezolizumab (Roche), and PD-L1 inhibitors such as atezolizumab, durvalumab, and avelumab. In some embodiments, the therapeutic agent(s) to use in combination is anti-IL-1 (e.g., anakinra), T cell activation inhibitors (e.g., dasatinib), JAK inhibitors (e.g., filgotinib), anti-GM-CSF (e.g., lenzilumab), anti-TNF (e.g., infliximab), Ang2 inhibitors (e.g., azilsartan), anti-angiogenic therapies (e.g., bevacizumab), and/or anti-IFNg (e.g., emapalumab-lzsg).

Additional therapeutic agents suitable for use in combination (before, after, and/or concurrently with T cell administration) with the compositions and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib), inhibitors of GM-CSF, CSF1, GM-CSFR, or CSF1R, in addition to anti-thymocyte globulin, lenzilumab and mavrilimumab.

In one embodiment, the GM-CSF inhibitor is selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule. In one embodiment, the CSF1 inhibitor is selected from RG7155, PD-0360324, MCS110/lacnotuzumab), or a biosimilar version of any one of the same; and a small molecule. In one embodiment, the GM-CSFR inhibitor and the CSF1R inhibitor is/are selected from Mavrilimumab (formerly CAM-3001; MedImmune, Inc.); cabiralizumab (Five Prime Therapeutics); LY3022855 (IMC-CS4)(Eli Lilly), Emactuzumab, also known as RG7155 or RO5509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule.

In some embodiments, a composition comprising an immunotherapy (e.g., engineered CAR T cells) is administered with an anti-inflammatory agent (before, after, and/or concurrently with T cell administration). Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In some embodiments, the compositions described herein are administered in conjunction with a cytokine (before, after, or concurrently with T cell administration). Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and —II; erythropoietin (EPO, Epogen®, Procrit®); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, the administration of the cells and the administration of the additional therapeutic agent are carried out on the same day, are carried out no more than 36 hours apart, no more than 24 hours apart, no more than 12 hours apart, no more than 6 hours apart, no more than 4 hours apart, no more than 2 hours apart, or no more than 1 hour apart or no more than 30 minutes apart. In some embodiments, the administration of the cells and the administration of the additional therapeutic agent are carried out between at or about 0 and at or about 48 hours, between at or about 0 and at or about 36 hours, between at or about 0 and at or about 24 hours, between at or about 0 and at or about 12 hours, between at or about 0 and at or about 6 hours, between at or about 0 and at or about 2 hours, between at or about 0 and at or about 1 hours, between at or about 0 and at or about 30 minutes, between at or about 30 minutes and at or about 48 hours, between at or about 30 minutes and at or about 36 hours, between at or about 30 minutes and at or about 24 hours, between at or about 30 minutes and at or about 12 hours, between at or about 30 minutes and at or about 6 hours, between at or about 30 minutes and at or about 4 hours, between at or about 30 minutes and at or about 2 hours, between at or about 30 minutes and at or about 1 hour, between at or about 1 hours and at or about 48 hours, between at or about 1 hour and at or about 36 hours, between at or about 1 hour and at or about 24 hours, between at or about 1 hour and at or about 12 hours, between at or about 1 hour and at or about 6 hours, between at or about 1 hour and at or about 4 hours, between at or about 1 hour and at or about 2 hours, between at or about 2 hours and at or about 48 hours, between at or about 2 hours and at or about 36 hours, between at or about 2 hours and at or about 24 hours, between at or about 2 hours and at or about 12 hours, between at or about 2 hours and at or about 6 hours, between at or about 2 hours and at or about 4 hours, between at or about 4 hours and at or about 48 hours, between at or about 4 hours and at or about 36 hours, between at or about 4 hours and at or about 24 hours, between at or about 4 hours and at or about 12 hours, between at or about 4 hours and at or about 6 hours, between at or about 6 hours and at or about 48 hours, between at or about 6 hours and at or about 36 hours, between at or about 6 hours and at or about 24 hours, between at or about 6 hours and at or about 12 hours, between at or about 12 hours and at or about 48 hours, between at or about 12 hours and at or about 36 hours, between at or about 12 hours and at or about 24 hours, between at or about 24 hours and at or about 48 hours, between at or about 24 hours and at or about 36 hours or between at or about 36 hours and at or about 48 hours. In some embodiments, the cells and the additional therapeutic agent are administered at the same time.

In some embodiments, the agent is administered in a dosage amount of from or from about 30 mg to 5000 mg, such as 50 mg to 1000 mg, 50 mg to 500 mg, 50 mg to 200 mg, 50 mg to 100 mg, 100 mg to 1000 mg, 100 mg to 500 mg, 100 mg to 200 mg, 200 mg to 1000 mg, 200 mg to 500 mg or 500 mg to 1000 mg.

In some embodiments, the agent is administered in a dosage amount from 0.5 mg/kg to 100 mg/kg, 1 mg/kg to 50 mg/kg, 1 mg kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered in a dosage amount from 1 mg/kg to 10 mg/kg, 2 mg kg/to 8 mg/kg, 2 mg/kg to 6 mg/kg, 2 mg/kg to 4 mg/kg or 6 mg/kg to 8 mg/kg, each In some aspects, the agent is administered in a dosage amount of at least 1 mg/kg, 2 mg/kg, 4 mg/kg, 6 mg/kg, 8 mg/kg, 10 mg/kg or more.

In some embodiments, the agent(s) is/are administered by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, the treatment further comprises bridging therapy, which is therapy between conditioning and the compositions disclosed herein or therapy administered after leukapheresis and completed prior to initiating conditioning chemotherapy. In some embodiments, the bridging therapy comprises, CHOP, G-CHOP, R-CHOP (rituximab, cyclophosphamide, doxorubicin, vincristine, and prednisolone), corticosteroids, bendamustine, platinum compounds, anthracyclines, and/orphosphoinositide 3-kinase (PI3K) inhibitors. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032). In some embodiments, the AKT inhibitor is perifosine, MK-2206. In one embodiment, the mTOR inhibitor is selected from everolimus, sirolimus, temsirolimus, ridaforolimus. In some embodiments, the dual PI3K/mTOR inhibitor is selected from BEZ235, XL765, and GDC-0980. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032).

In some embodiments, the bridging therapy comprises acalabrutinib, brentuximab vedotin, copanlisib hydrochloride, nelarabine, belinostat, bendamustine hydrochloride, carmustine, bleomycin sulfate, bortezomib, zanubrutinib, carmustine, chlorambucil, copanlisib hydrochloride, denileukin diftitox, dexamethasone, doxorubicin hydrochloride, duvelisib, pralatrexate, obinutuzumab, ibritumomab tiuxetan, ibrutinib, idelalisib, recombinant interferon alfa-2b, romidepsin, lenalidomide, mechloretamine hydrochloride, methotrexate, mogamulizumab-kpc, prerixafor, nelarabine, obinutuzumab, denileukin diftitox, pembrolizumab, plerixafor, polatuzumab vedotin-piiq, mogamulizumab-kpc, prednisone, rituximab, hyaluronidase, romidepsin, bortezomib, venetoclax, vinblastine sulfate, vorinostat, zanubrutinib, CHOP, COPP, CVP, EPOCH, R-EPOCH, HYPER-CVAD, ICE, R-ICE, R-CHOP, R-CVP, and combinations of the same.

In some embodiments, the cell immunotherapy is administered in conjunction with debulking therapy, which is used with the aim of reducing tumor burden. In one embodiment, debulking therapy is to be administered after leukapheresis and prior to administration of conditioning chemotherapy or cell infusion. Examples of debulking therapy include the following (Table 1).

TABLE 1
Exemplary debulking bridging therapies

| Type | Proposed Regimen^{a} | Timing/Washout |
|---|---|---|
| R-CHOP | Rituximab 375 mg/m² Day 1 | Should be administered after |
| | Doxorubicin 50 mg/m² Day 1 | leukapheresis/enrollment and |
| | Prednisone 100 mg Day 1 | should be completed |
| | through Day 5 | at least 14 days prior to the |
| | Cyclophosphamide 750 | start of conditioning |
| | mg/m² Day 1 Vincristine 1.4 | chemotherapy |
| | mg/m² Day 1 | |
| R-ICE | Rituximab 375 mg/m² Day 1 | |
| | Ifosfamide 5 g/m² 24 h-CI | |
| | Day 2 | |
| | Carboplatin AUC5 Day 2 | |
| | maximum dose 800 mg | |
| | Etoposide 100 mg/m²/d Days | |
| | 1 through Day 3 | |
| R-GEMOX | Rituximab 375 mg/m² Day 1 | |
| | Gemcitabine 1000 mg/m² | |
| | Day 2 Oxaliplatin 100 mg/m² | |
| | Day 2 | |
| R-GDP | Rituximab 375 mg/m² Day 1 | |
| | (or Day 8) | |
| | Gemcitabine 1 g/m² on Day 1 | |
| | and Day 8 Dexamethasone 40 | |
| | mg on Day 1 through Day 4 | |
| | Cisplatin 75 mg/m² on Day 1 | |
| | (or carboplatin AUC5 on Day 1) | |
| RADIOTHERAPY^{b} | Per local standard up to 20 to | Should be administered after |
| | 30 Gy | leukapheresis/enrollment and |
| | | should be completed |
| | | at least 5 days prior to the start |
| | | of conditioning chemotherapy |

Abbreviations: AUC, area under the curve
^{a}Other debulking treatment options may be used, to be discussed with the medical monitor. Supportive care with hydration, anti-emesis, mesna, growth factor support, and tumor lysis prophylaxis according to local standard may be used. More than 1 cycle allowed.
^{b}At least 1 target lesion should remain outside of the radiation field to allow for tumor measurements

### Monitoring

In some embodiments, administration of the immunotherapy (e.g., chimeric receptor T cell immunotherapy) occurs at a certified healthcare facility.

In some embodiments, the methods disclosed herein comprise monitoring patients at least daily for 7 days at the certified healthcare facility following infusion for signs and symptoms of CRS and neurologic toxicities and other adverse reactions to CAR T cell treatment. In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

### Prevention or Management of Adverse Events

In some embodiments, the method comprises management of adverse events in any subject. The terms “adverse events,” “adverse reaction,” and “adverse effect” are used interchangeably herein. In some embodiments, the adverse event is selected from the group consisting of cytokine release syndrome (CRS), a neurologic toxicity, a hypersensitivity reaction, a serious infection, a cytopenia and hypogammaglobulinemia.

In some embodiments, the present disclosure provides methods of preventing the development or reducing the severity of adverse events based on the levels of a number of biomarker levels in the serum of the subject undergoing immunotherapy. In some embodiments, the cell therapy is administered in with one or more agents that prevents, delays the onset of, reduces the symptoms of, treats the adverse events, which include cytokine release syndromes and neurologic toxicity. In one embodiment, the agent has been described above. In other embodiments, the agent is described below. In some embodiments, the agent is administered by one of the methods and doses described elsewhere in the specification, before, after, or concurrently with the administration of the cells. In one embodiment, the agent(s) are administered to a subject that may be predisposed to the disease but has not yet been diagnosed with the disease.

In some embodiments, the immunotherapy (e.g., cell treatment) is administered before, during/concurrently, and/or after the administration of one or more agents (e.g., steroids) or treatments (e.g., debulking) that treat and or prevent (are prophylactic) one or more symptoms of adverse events. The pharmacologic and/or physiologic effect may be prophylactic, i.e., the effect completely or partially prevents a disease or symptom thereof. A “prophylactically effective amount” refers to an amount effective, at dosages and for periods of time necessary, to achieve the desired prophylactic result. In one embodiment, a prophylactically effective amount is used in subjects prior to or at an earlier stage of disease. In one embodiment, the prophylactically effective amount will be less than the therapeutically effective amount. In some embodiments, the patient is selected for management of adverse events based on the expression of one of more of the markers described herein in this specification. In one embodiment, the adverse event treatment or prophylaxis is administered to any patient that will receive, is receiving, or has received cell therapy.

In some embodiments, the signs and symptoms of adverse reactions are selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia.

In some embodiments, the patient has been identified and selected based on one or more of the biomarkers described in this application. In some embodiments, the patient has been identified and selected simply by the clinical presentation (e.g., presence and grade of toxicity symptom).

In some embodiments, the adverse events/reactions may be chosen from one or more of the following (Table 2):

TABLE 2
Exemplary adverse events
Adverse Event/Reaction

Blood and Lymphatic System Disorders
Coagulopathy
Cardiac Disorders
Tachycardias
Bradycardias
Non-ventricular Arrhythmias
Gastrointestinal Disorders
Nausea
Constipation
Diarrhea
Abdominal pain
Oral pain
Vomiting
Dysphagia
Pyrexia
Fatigue
Chills
Edema
Dry mouth
Pain
Immune System Disorders
Cytokine release syndrome
Hypogammaglobulinemia
Infections and Infestations
Infection - pathogen unspecified
Viral infections
Bacterial infections
Metabolism and nutrition disorders
Decreased appetite
Musculoskeletal pain
Motor dysfunction
Psychiatric Disorders
Nervous System Disorders
Encephalopathy
Tremor
Headache
Aphasia
Dizziness
Neuropathy
Insomnia
Delirium
Anxiety
Immune System Disorders
Cytokine release syndrome
Hypogammaglobulinemia
Infections and Infestations
Infection - pathogen unspecified
Viral infections
Bacterial infections
Metabolism and nutrition disorders
Decreased appetite
Musculoskeletal pain
Motor dysfunction
Psychiatric Disorders
Nervous System Disorders
Encephalopathy
Tremor
Headache
Aphasia
Dizziness
Neuropathy
Insomnia
Delirium
Anxiety
Renal and Urinary Disorders
Renal insufficiency
Urine output decreased
Hypoxia
Cough
Dyspnea
Pleural effusion
Skin and Subcutaneous Tissue Disorders
Rash
Vascular Disorders
Hypotension
Hypertension
Thrombosis
Hemorrhage

### Cytokine Release Syndrome (CRS)

In some embodiments, the method comprises preventing or reducing the severity of CRS in a chimeric receptor treatment. In some embodiments, the engineered CAR T cells are deactivated after administration to the patient.

In some embodiments, the method comprises identifying CRS based on clinical presentation. In some embodiments, the method comprises evaluating for and treating other causes of fever, hypoxia, and hypotension. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered.

In some embodiments, the method comprises monitoring patients at least daily for 7 days at the certified healthcare facility following infusion for signs and symptoms of CRS. In some embodiments, the method comprises monitoring patients for signs or symptoms of CRS for 4 weeks after infusion. In some embodiments, the method comprises counseling patients to seek immediate medical attention should signs or symptoms of CRS occur at any time. In some embodiments, the method comprises instituting treatment with supportive care, tocilizumab or tocilizumab and corticosteroids as indicated at the first sign of CRS.

In some embodiments, the subject experiences Grade 3+ CRS. In some embodiments, this includes pyrexia, hypotension, tachycardia, hypoxia, chills, sinus tachycardia, fatigue, headache, vomiting, acute kidney injury, myalgia, atrial fibrillation, diarrhea, dyspnea, ejection fraction decreased, pulmonary oedema, atrial flutter, blood creatine increased, capillary leak syndrome, decreased appetite, febrile neutropenia, malaise, metabolic acidosis, fever, nausea, headache, rash, rapid heartbeat, low blood pressure, trouble breathing, etc.

### Neurologic Toxicity (NT)

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the subject experiences Grade 3+ NT. In some embodiments, this includes encephalopathy, tremor, confusional state, aphasia, somnolence, agitation, memory impairment, dysarthria, hallucination, mental status changes, ataxia, restlessness, seizure, delirium, disturbance in attention, lethargy, depressed level of consciousness, disorientation, dyscalculia, hemiparesis, monoclonus, cerebral edema, and others.

### Management of Adverse Events

In some embodiments, the method of managing adverse events comprises monitoring patients at least daily for 7 days at the certified healthcare facility following infusion for signs and symptoms of neurologic toxicities. In some embodiments, the method comprises monitoring patients for signs or symptoms of neurologic toxicities and/or CRS or 4 weeks after infusion.

In some embodiments, the disclosure provides two methods of managing adverse events in subjects receiving CAR T cell treatment with steroids and anti-IL6/anti-IL-6R antibody/ies. In one embodiment, the CAR T cell treatment is an anti-CD19 treatment, as described in the Examples. In one embodiment, the CAR T cell treatment is known as ZUMA-1, which includes different adverse event management protocols for different cohorts. In one embodiment, the disclosure provides that early steroid intervention in Cohort 4 is associated with lower rates of severe CRS and neurologic events than what was observed in Cohorts 1+2. In one embodiment, the disclosure provides that earlier use of steroids in Cohort 4 was associated with a median cumulative cortisone-equivalent dose approximately 15% of that in Cohorts 1+2, suggesting that earlier steroid use may allow reduction of overall steroid exposure. Accordingly, in one embodiment, the disclosure provides a method of adverse event management whereby corticosteroid therapy is initiated for management of all cases of grade 1 CRS if there was no improvement after 3 days and for all grade≥1 neurologic events. In one embodiment, tocilizumab is initiated for all cases of grade 1 CRS if there is no improvement after 3 days and for all grade≥2 neurologic events. In one embodiment, the disclosure provides a method of reducing overall steroid exposure in patients receiving adverse event management after CAR T cell administration, the method comprising initiation of corticosteroid therapy for management of all cases of grade 1 CRS if there was no improvement after 3 days and for all grade≥1 neurologic events and/or initiation of tocilizumab for all cases of grade 1 CRS if there is no improvement after 3 days and for all grade≥2 neurologic events. In one embodiment, the corticosteroid and tocilizumab are administering in a regimen selected from those exemplified in protocols A through C. In one embodiment, the disclosure provides that earlier steroid use is not associated with increased risk for severe infection, decreased CAR T-cell expansion, or decreased tumor response.

In one embodiment, the disclosure supports the safety of levetiracetam prophylaxis in CAR T cell cancer treatment. In one embodiment, the cancer is NHL. In one embodiment, the cancer is R/R LBCL and the patients receive axicabtagene ciloleucel. Accordingly, in one embodiment, the disclosure provides a method of managing adverse events in patients treated with CAR T cells comprising administering to the patient a prophylactic dosage of an anti-seizure medication. In some embodiments, the patients receive levetiracetam (for example, 750 mg orally or intravenous twice daily) starting on day 0 of the CAR T cell treatment (after conditioning) and also at the onset of grade≥2 neurologic toxicities, if neurologic events occur after the discontinuation of prophylactic levetiracetam. In one embodiment, if a patient does not experience any grade≥2 neurologic toxicities, levetiracetam is tapered and discontinued as clinically indicated. In one embodiment, levetiracetam prophylaxis is combined with any other adverse event management protocol.

In one embodiment, the disclosure provides that CAR T-cell levels in the patients subject to the adverse management protocol of Cohort 4 were comparable to those of Cohorts 1+2. In one embodiment, the disclosure provides that the numerical levels of key inflammatory cytokines associated with CAR-related inflammatory events (e.g., IFNγ, IL-2 and GM-CSF) are lower in Cohort 4 than in Cohorts 1+2. Accordingly, the disclosure provides a method of reducing CAR T cell treatment-related inflammatory events without impact on CAR T cell levels comprising administering to the patient the adverse event management protocol of Cohort 4. The disclosure also provides a method of reducing cytokine production by immune cells after CAR T cell therapy comprising administering to the patient the adverse event management protocol of Cohort 4. In one embodiment, this effect is obtained without affecting CAR T-cell expansion and response rates. In one embodiment, the patient has R/R LBCL. In one embodiment, the CAR T cell treatment is anti-CD19 CAR T cell treatment. In one embodiment, the CAR T cell treatment comprises axicabtagene ciloleucel.

In one embodiment, the disclosure provides that early or prophylactic use of tocilizumab following axicabtagene ciloleucel for adverse event management decreased grade≥3 cytokine release syndrome but increased grade≥3 neurologic events. Accordingly, the disclosure provides a method for adverse event management in CAR T-cell therapy. In one embodiment, patients receive levetiracetam (750 mg oral or intravenous twice daily) starting on day 0. At the onset of grade≥2 neurologic events, levetiracetam dose is increased to 1000 mg twice daily. If a patient did not experience any grade≥2 neurologic event, levetiracetam is tapered and discontinued as clinically indicated. Patients also receive tocilizumab (8 mg/kg IV over 1 hour [not to exceed 800 mg]) on day 2. Further tocilizumab (±corticosteroids) may be recommended at the onset of grade 2 CRS in patients with comorbidities or older age, or otherwise in case of grade≥3 CRS. For patients experiencing grade≥2 neurologic events, tocilizumab is initiated, and corticosteroids are added for patients with comorbidities or older age, or if there is any occurrence of a grade≥3 neurologic event with worsening symptoms despite tocilizumab use.

In one embodiment, the disclosure provides that prophylactic steroid use appears to reduce the rate of severe CRS and NEs to a similar extent as early steroid use following axicabtagene ciloleucel administration. Accordingly, the disclosure provides a method for adverse event management in CAR T-cell therapy wherein patients receive dexamethasone 10 mg PO on Days 0 (prior to axicabtagene ciloleucel infusion), 1, and 2. Steroids are also administered starting at Grade 1 NE, and for Grade 1 CRS when no improvement is observed after 3 days of supportive care. Tocilizumab is also administered for Grade≥1 CRS if no improvement is observed after 24 hours of supportive care.

In one embodiment, the disclosure provides that adverse event management of CAR T-cell therapy with an antibody that neutralizes and/or depletes GM-CSF prevents or reduces treatment-related CRS and/or NEs in treated patients. In one embodiment, the antibody is lenzilumab.

In one embodiment, the method of prevention and/or management of adverse events comprises administering a “prophylactically effective amount” of tocilizumab, of a corticosteroid therapy, and/or of an anti-seizure medicine for toxicity prophylaxis. In some embodiments, the method comprises administering inhibitors of GM-CSF, CSF1, GM-CSFR, or CSF1R, lenzilumab, mavrilimumab, cytokines, and/or anti-inflammatory agents.

In some embodiments, the adverse events are managed by the administration of an agent/agents that is/are an antagonist or inhibitor of IL-6 or the IL-6 receptor (IL-6R). In some embodiments, the agent is an antibody that neutralizes IL-6 activity, such as an antibody or antigen-binding fragment that binds to IL-6 or IL-6R. For example, in some embodiments, the agent is or comprises tocilizumab (atlizumab) or sarilumab, anti-IL-6R antibodies. In some embodiments, the agent is an anti-IL-6R antibody described in U.S. Pat. No. 8,562,991. In some cases, the agent that targets IL-6 is an anti-TL-6 antibody, such as siltuximab, elsilimomab, ALD518/BMS-945429, sirukumab (CNTO 136), CPSI-2634, ARGX 109, FE301, FM101, or olokizumab (CDP6038), and combinations thereof. In some embodiments, the agent may neutralize IL-6 activity by inhibiting the ligand-receptor interactions. In some embodiments, the IL-6/IL-6R antagonist or inhibitor is an IL-6 mutein, such as one described in U.S. Pat. No. 5,591,827. In some embodiments, the agent that is an antagonist or inhibitor of IL-6/IL-6R is a small molecule, a protein or peptide, or a nucleic acid.

In some embodiments, other agents that may be used to manage adverse reactions and their symptoms include an antagonist or inhibitor of a cytokine receptor or cytokine. In some embodiments, the cytokine or receptor is IL-10, TL-6, TL-6 receptor, IFNy, IFNGR, IL-2, IL-2R/CD25, MCP-1, CCR2, CCR4, MIP13, CCR5, TNFalpha, TNFR1, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCR5), TNF alpha receptor (e.g., TNFR1), IL-1 receptor (IL1-Ra/IL-1RP), or IL-10 receptor (IL-10R), IL-1, and IL-1Ralpha/IL-1beta. In some embodiments, the agent comprises situximab, sarilumab, olokizumab (CDP6038), elsilimomab, ALD518/BMS-945429, sirukumab (CNTO 136), CPSI-2634, ARGX 109, FE301, or FM101. In some embodiments, the agent, is an antagonist or inhibitor of a cytokine, such as transforming growth factor beta (TGF-beta), interleukin 6 (TL-6), interleukin 10 (IL-10), IL-2, MIP13 (CCL4), TNF alpha, IL-1, interferon gamma (IFN-gamma), or monocyte chemoattractant protein-I (MCP-1). In some embodiments, the is one that targets (e.g. Inhibits or is an antagonist of) a cytokine receptor, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCR5), TNF alpha receptor (e.g., TNFR1), IL-1 receptor (IL1-Ra/IL-1RP), or IL-10 receptor (IL-10R) and combinations thereof. In some embodiments, the agent is administered by one of the methods and doses described elsewhere in the specification, before, after, or concurrently with the administration of the cells.

In some embodiments, the agent is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or the agent is administered in a dosage amount of at least or at least about or about 2 mg/kg, 4 mg/kg, 6 mg/kg or 8 mg/kg. In some embodiments, is administered in a dosage amount from about 1 mg/kg to 12 mg/kg, such as at or about 10 mg/kg. In some embodiments, the agent is administered by intravenous infusion. In one embodiment, the agent is tocilizumab. In some embodiments, the (agent(s), e.g., specifically tocilizumab) is/are administered by one of the methods and doses described elsewhere in the specification, before, after, or concurrently with the administration of the cells.

In some embodiments, the method comprises identifying CRS based on clinical presentation. In some embodiments, the method comprises evaluating for and treating other causes of fever, hypoxia, and hypotension. If CRS is observed or suspected, it may be managed according to the recommendations in protocol A, which may also be used in combination with the other treatments of this disclosure, including Neutralization or Reduction of the CSF/CSFR1 Axis. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered. In some embodiments, a biosimilar or equivalent of tocilizumab may be used instead of tocilizumab in the methods disclosed herein. In other embodiments, another anti-IL6R may be used instead of tocilizumab.

In some embodiments, adverse events are managed according to the following protocol (protocol A/Table 3):

TABLE 3
CRS grading and management guidance

| CRS Grade (a) | Tocilizumab | Corticosteroids |
|---|---|---|
| Grade 1 | If symptoms (e.g., fever) not | If not improving after 3 days, |
| Symptoms require | improving after 24 hours, | administer one dose of |
| symptomatic treatment only | consider managing as Grade | dexamethasone 10 mg |
| (e.g., fever, nausea, fatigue, | 2. | intravenously. |
| headache, myalgia, malaise). | | |
| Grade 2 | Administer tocilizumab c 8 | Administer dexamethasone 10 |
| Symptoms require and | mg/kg intravenously over 1 | mg intravenously once daily. |
| respond to moderate | hour (not to exceed 800 mg). | If improving, manage as |
| intervention. | If no clinical improvement in | Grade 1 above and continue |
| Oxygen requirement less | the signs and symptoms of | corticosteroids until the |
| than 40% FiO₂ or | CRS after the first dose, | severity is Grade 1 or less, |
| hypotension responsive to | repeat tocilizumab every 8 | then quickly taper as clinically |
| fluids or low-dose of one | hours as needed. | appropriate. |
| vasopressor or Grade 2 organ | Limit to a maximum of 3 | If not improving, manage as |
| toxicity (b). | doses in a 24-hour period; | appropriate grade below. |
| | maximum total of 4 doses. | |
| | If improving, discontinue | |
| | tocilizumab. | |
| Grade 3 | Per Grade 2 | Dexamethasone 10 mg |
| Symptoms require and | If improving, manage as | intravenously three times a |
| respond to aggressive | appropriate grade above | day. |
| intervention. | | If improving, manage as |
| Oxygen requirement greater | | appropriate grade above and |
| than or equal to 40% FiO₂ or | | continue corticosteroids until |
| hypotension requiring high- | | the severity is Grade 1 or less, |
| dose or multiple vasopressors | | then quickly taper as clinically |
| or Grade 3 organ toxicity or | | appropriate. |
| Grade 4 transaminitis. | | If not improving, manage as |
| | | Grade 4. |
| Grade 4 | Per Grade 2 | Administer |
| Life-threatening symptoms. | If improving, manage as | methylprednisolone 1000 mg |
| Requirements for ventilator | appropriate grade above. | intravenously once per day for |
| support, continuous veno- | | 3 days. |
| venous hemodialysis | | If improving, manage as |
| (CVVHD) or | | appropriate grade above and |
| Grade 4 organ toxicity | | continue corticosteroids until |
| (excluding transaminitis). | | the severity is Grade 1 or less, |
| | | then taper as clinically |
| | | appropriate. |
| | | If not improving, consider |
| | | methylprednisolone 1000 mg |
| | | 2-3 times a day or alternate |
| | | therapy. |

(a) Lee D W et al., (2014). Current concepts in the diagnosis and management of cytokine release syndrome. Blood. 2014 Jul. 10; 124(2): 188-195.
(b) Refer to Procotocol B for management of neurologic toxicity.
(c) Refer to ACEMTRA ® (tocilizumab) Prescribing Information for details, https://www.gene.com/download/pdf/actemra_prescribing.pdf (last accessed Oct. 18, 2017). Initial U.S. approval is indicated to be in 2010.
(d) Alternate therapy includes (but is not limited to): anakinra, siltuximab, ruxolitinib, cyclophosphamide, IVIG and ATG.

### Neurologic Toxicity

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. Consider non-sedating, anti-seizure medicines (e.g., levetiracetam) for seizure prophylaxis for any ≥Grade 2 neurologic toxicities. The following treatments may be used in combination with the other treatments of this disclosure, including Neutralization or Reduction of the CSF/CSFR1 Axis.

In some embodiments, NE are managed according to the following protocol (protocol B/Table 4):

TABLE 4
Neurologic toxicity grading and management guidance

| Grading | | |
|---|---|---|
| Assessment | Concurrent CRS | No concurrent CRS |
| Grade 1 | Administer tocilizumab per protocol A | Administer one dose of |
| | for management of Grade 1 CRS. | dexamethasone 10 mg |
| | In addition, administer one dose of | intravenously. |
| | dexamethasone 10 mg intravenously. | If not improving after 2 days, |
| | If not improving after 2 days, repeat | repeat dexamethasone 10 mg |
| | dexamethasone 10 mg intravenously. | intravenously. |
| | Consider levetiracetam for seizure | Consider levetiracetam for |
| | prophylax | seizure prophylax |
| Grade 2 | Administer tocilizumab per Table 3 for | Administer dexamethasone 10 |
| | management of Grade 2 CRS. | mg intravenously four times a |
| | In addition, administer dexamethasone 10 | day. |
| | mg intravenously four times a day. | If improving, continue |
| | If improving, continue corticosteroids | corticosteroids until the |
| | until the severity is Grade 1 or less, then | severity is Grade 1 or less, |
| | quickly taper as clinically appropriate. | then quickly taper as clinically |
| | If not improving, manage as appropriate | appropriate. |
| | grade below. | If not improving, manage as |
| | | appropriate grade below. |
| | Consider non-sedating, anti-seizure medicines (e.g., levetiracetam) for | |
| | seizure prophylaxis. | |
| Grade 3 | Administer tocilizumab per (protocol | Administer |
| | A/Table 3) for management of Grade 2 | methylprednisolone 1000 mg |
| | CRS. | intravenously once daily. |
| | In addition, administer | If improving, manage as |
| | methylprednisolone 1000 mg | appropriate grade above and |
| | intravenously once daily. | continue corticosteroids until |
| | If improving, manage as appropriate | the severity is Grade 1 or less, |
| | grade above and continue corticosteroids | then taper as clinically |
| | until the severity is Grade 1 or less, then | appropriate. |
| | taper as clinically appropriate. | If not improving, manage as |
| | If not improving, manage as Grade 4. | Grade 4. |
| | Consider non-sedating, anti-seizure medicines (e.g., levetiracetam) for | |
| | seizure prophylaxis. | |
| Grade 4 | Administer tocilizumab per Table 3 for | Administer |
| | management of Grade 2 CRS. | methylprednisolone 1000 mg |
| | In addition, administer | intravenously twice per day. |
| | methylprednisolone 1000 mg | If improving, manage as |
| | intravenously twice per day. | appropriate grade above and |
| | If improving, manage as appropriate | continue corticosteroids until |
| | grade above and continue corticosteroids | the severity is Grade 1 or less, |
| | until the severity is Grade 1 or less, then | then taper as clinically |
| | taper as clinically appropriate. | appropriate. |
| | If not improving, consider 1000 mg of | If not improving, consider |
| | methylprednisolone intravenously 3 times | 1000 mg of |
| | a day or alternate therapy.b | methylprednisolone |
| | | intravenously 3 times a day or |
| | | alternate therapy.b |

Consider non-sedating, anti-seizure medicines (e.g., levetiracetam) for
seizure prophylaxis.
^{a} Severity based on Common Terminology Criteria for Adverse Events.
bAlternate therapy includes (but is not limited to): anakinra, siltuximab, ruxolitinib, cyclophosphamide, IVIG and ATG.
methylprednisolone may be substituted for equivalent levels of dexamethasone.

Additional Safety Management Strategies with Corticosteroids

Administration of corticosteroids and/or tocilizumab at Grade 1 may be considered prophylactic. Supportive care may be provided in all protocols at all CRS and NE severity grades.

In one embodiment of a protocol for management of adverse events related to CRS, tocilizumab and/or corticosteroids are administered as follows: Grade 1 CRS: no tocilizumab; no corticosteroids; Grade 2 CRS: tocilizumab (only in case of comorbidities or older age); and/or corticosteroids (only in case of comorbidities or older age); Grade 3 CRS: tocilizumab; and/or corticosteroids; Grade 4 CRS: tocilizumab; and/or corticosteroids. In another embodiment of a protocol for management of adverse events related to CRS, tocilizumab and/or corticosteroids are administered as follows: Grade 1 CRS: tocilizumab (if no improvement after 3 days); and/or corticosteroids (if no improvement after 3 days); Grade 2 CRS: tocilizumab; and/or corticosteroids; Grade 3 CRS: tocilizumab; and/or corticosteroids; Grade 4 CRS: tocilizumab; and/or corticosteroids, high dose.

In one embodiment of a protocol for management of adverse events related to NE, tocilizumab and/or corticosteroids are administered as follows: Grade 1 NE: no tocilizumab; no corticosteroids;

Grade 2 NE: no tocilizumab; no corticosteroids; Grade 3 NE: tocilizumab; and/or corticosteroids (only if no improvement to tocilizumab, standard dose); Grade 4 NE: tocilizumab; and/or corticosteroids.

In another embodiment of a protocol for management of adverse events related to NE, tocilizumab and/or corticosteroids are administered as follows: Grade 1 NE: no tocilizumab; and/or corticosteroids; Grade 2 NE: tocilizumab; and/or corticosteroids; Grade 3 NE: tocilizumab; and/or corticosteroids, high dose; Grade 4 NE: tocilizumab; and/or corticosteroids, high dose.

In one embodiment, corticosteroid treatment is initiated at CRS grade≥2 and tocilizumab is initiated at CRS grade 2. In one embodiment, corticosteroid treatment is initiated at CRS grade 1 and tocilizumab is initiated at CRS grade 1. In one embodiment, corticosteroid treatment is initiated at NE grade≥3 and tocilizumab is initiated at CRS grade≥3. In one embodiment, corticosteroid treatment is initiated at CRS grade≥1 and tocilizumab is initiated at CRS grade M 2. In some embodiments, prophylactic use of tocilizumab administered on Day 2 may decrease the rates of Grade≥3 CRS.

In one embodiment, the protocol for treatment of adverse events comprises Protocol C, as follows (Table 5).

TABLE 5
Alternative adverse management guidance

| CRS Grade | Tocilizumab Dose^{a} | Corticosteroid Dose^{a} |
|---|---|---|
| 1 | 8 mg/kg over 1 hour^{b} if no | Dexamethasone 10 mg × 1 |
| | improvement after 24 hours of | if no improvement after 3 days |
| | supportive care; repeat every 4-6 | |
| | hours as needed | |
| 2 | 8 mg/kg over 1 hour^{b}; repeat | Dexamethasone 10 mg × 1 |
| | every 4-6 hours as needed | |
| 3 | Per Grade 2 | Methylprednisolone 1 mg/kg IV |
| | | twice daily or equivalent |
| | | dexamethasone dose |
| 4 | Per Grade 2 | Methylprednisolone 1000 mg/d IV |
| | | for 3 days |

| NE Grade | Tocilizumab Dose | Corticosteroid Dose |
|---|---|---|
| 1 | N/A | Dexamethasone 10 mg × 1 |
| 2 | Only in the case of concurrent | Dexamethasone 10 mg 4×/day |
| | CRS; 8 mg/kg over 1 hour; | |
| | repeat every 4-6 hours as needed | |
| 3 | Per Grade 2 | Methylprednisolone 1 g once daily |
| 4 | Per Grade 2 | Methylprednisolone 1 g twice |
| | | daily |

^{a}Therapy to be tapered on improvement of symptoms at investigator's discretion
^{b}Not to exceed 800 mg; AE, adverse event; CRS, cytokine release syndrome; IV, intravenous; N/A, not applicable; NE, neurologic event

Any corticosteroid may be appropriate for this use. In one embodiment, the corticosteroid is dexamethasone. In some embodiments, the corticosteroid is methylprednisolone. In some embodiments, the two are administered in combination. In some embodiments, glucocorticoids include synthetic and non-synthetic glucocorticoids. Exemplary glucocorticoids include, but are not limited to: alclomethasones, algestones, beclomethasones (e.g., beclomethasone dipropionate), betamethasones (e.g., betamethasone 17 valerate, betamethasone sodium acetate, betamethasone sodium phosphate, betamethasone valerate), budesonides, clobetasols (e.g., clobetasol propionate), clobetasones, clocortolones (e.g., clocortolone pivalate), cloprednols, corticosterones, cortisones and hydrocortisones (e.g., hydrocortisone acetate), cortivazols, deflazacorts, desonides, desoximethasones, dexamethasones (e.g., dexamethasone 21-phosphate, dexamethasone acetate, dexamethasone sodium phosphate), diflorasones (e.g., diflorasone diacetate), diflucortolones, difluprednates, enoxolones, fluazacorts, flucloronides, fludrocortisones (e.g., fludrocortisone acetate), flumethasones (e.g., flumethasone pivalate), flunisolides, fluocinolones (e.g., fluocinolone acetonide), fluocinonides, fluocortins, fluocortolones, fluorometholones (e.g., fluorometholone acetate), fluperolones (e.g., fluperolone acetate), fluprednidenes, fluprednisolones, flurandrenolides, fluticasones (e.g., fluticasone propionate), formocortals, halcinonides, halobetasols, halometasones, halopredones, hydrocortamates, hydrocortisones (e.g., hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylpredni solones (methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g., prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g., triamcinolone acetonide, triamcinolone benetonide, triamcinolone hexacetonide, triamcinolone acetonide 21 palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980) and Remington: The Science and Practice of Pharmacy, 22nd Edition, Lippincott Williams & Wilkins, Philadelphia, Pa. (2013) and any other editions, which are hereby incorporated by reference. In some embodiments, the glucocorticoid is selected from among cortisones, dexamethasones, hydrocortisones, methylprednisolones, prednisolones and prednisones. In an embodiment, the glucocorticoid is dexamethasone. In other embodiments, the steroid is a mineralcorticoid. Any other steroid may be used in the methods provided herein.

The one or more corticosteroids may be administered at any dose and frequency of administration, which may be adjusted to the severity/grade of the adverse event (e.g., CRS and NE). The tables above provide examples of dosage regimens for management of CRS and NE, respectively. In another embodiment, corticosteroid administration comprises oral or IV dexamethasone 10 mg, 1-4 times per day. Another embodiment, sometimes referred to as “high-dose” corticosteroids, comprises administration of IV methylprednisone 1 g per day alone, or in combination with dexamethasone. In some embodiments, the one or more cortico steroids are administered at doses of 1-2 mg/kg per day.

The corticosteroid may be administered in any amount that is effective to ameliorate one or more symptoms associated with the adverse events, such as with the CRS or neurotoxicity. The corticosteroid, e.g., glucocorticoid, may be administered, for example, at an amount between at or about 0.1 and 100 mg, per dose, 0.1 to 80 mg, 0.1 to 60 mg, 0.1 to 40 mg, 0.1 to 30 mg, 0.1 to 20 mg, 0.1 to 15 mg, 0.1 to 10 mg, 0.1 to 5 mg, 0.2 to 40 mg, 0.2 to 30 mg, 0.2 to 20 mg, 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg, 0.4 to 5 mg, 0.4 to 4 mg, 1 to 20 mg, 1 to 15 mg or 1 to 10 mg, to a 70 kg adult human subject. Typically, the corticosteroid, such as a glucocorticoid is administered at an amount between at or about 0.4 and 20 mg, for example, at or about 0.4 mg, 0.5 mg, 0.6 mg, 0.7 mg, 0.75 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg or 20 mg per dose, to an average adult human subject.

In some embodiments, the corticosteroid may be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

Generally, the dose of corticosteroid administered is dependent upon the specific corticosteroid, as a difference in potency exists between different corticosteroids. It is typically understood that drugs vary in potency, and that doses may therefore vary, in order to obtain equivalent effects. Equivalence in terms of potency for various glucocorticoids and routes of administration. is well known. Information relating to equivalent steroid dosing (in a non-chronotherapeutic manner) may be found in the British National Formulary (BNF) 37, March 1999.

In some embodiments, the adverse events are managed by the following protocol: patients receive levetiracetam (750 mg oral or intravenous twice daily) starting on day 0 of administration of T cell therapy; at the onset of grade≥2 neurologic events, levetiracetam dose is increased to 1000 mg twice daily; if a patient did not experience any grade≥2 neurologic event, levetiracetam is tapered and discontinued as clinically indicated; patients also receive tocilizumab (8 mg/kg IV over 1 hour [not to exceed 800 mg]) on day 2; further tocilizumab (±corticosteroids) may be recommended at the onset of grade 2 CRS in patients with comorbidities or older age, or otherwise in case of grade≥3 CRS; for patients experiencing grade≥2 neurologic events, tocilizumab is initiated, and corticosteroids are added for patients with comorbidities or older age, or if there is any occurrence of a grade≥3 neurologic event with worsening symptoms despite tocilizumab use. In some embodiments, levetiracetam is administered for prophylaxis and at the onset of grade≥2 neurologic toxicities, if neurologic events occur after the discontinuation of prophylactic levetiracetam and/or levetiracetam is tapered and discontinued if the patient does not experience any grade≥2 neurologic toxicities.

In some embodiments, the adverse events are managed by the following protocol: patients receive dexamethasone 10 mg PO on Days 0 (prior to T cell therapy infusion), 1, and 2; steroids are also administered starting at Grade 1 NE, and for Grade 1 CRS when no improvement is observed after 3 days of supportive care; tocilizumab is also administered for Grade≥1 CRS if no improvement is observed after 24 hours of supportive care.

### Secondary Malignancies

In some embodiments, patients treated with CAR T cells (e.g., CD19-directed) or other genetically modified autologous T cell immunotherapy may develop secondary malignancies. In certain embodiments, patients treated with CAR T cells (e.g., CD19-directed) or other genetically modified allogeneic T cell immunotherapy may develop secondary malignancies. In some embodiments, the method comprises monitoring life-long for secondary malignancies.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference. All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

The following examples are intended to illustrate various aspects of the application. As such, the specific aspects discussed are not to be construed as limitations on the scope of the application. For example, although the Examples below are directed to T cells transduced with an anti-CD19 chimeric antigen receptor (CAR), one skilled in the art would understand that the methods described herein may apply to immune cells transduced with any CAR. The methods are also applicable to other immunotherapies. It will be apparent to one skilled in the art that various equivalents, changes, and modifications may be made without departing from the scope of application, and it is understood that such equivalent aspects are to be included herein.

The disclosures provided by this application may be used in a variety of methods in additional to, or as a combination of, the methods described above. The following is a compilation of exemplary methods that may be derived from the disclosures provided in this application.

### EXAMPLES

### Example 1

High B-Cell Signature was Associated with Improved EFS and Higher Probability of Durable Response after Axi-Cel

To identify novel prognostic markers in LBCL, gene expression analysis of pretreatment tumor biopsies (collected either at initial diagnosis or before lymphodepleting chemotherapy) was performed and leveraged the NanoString PanCancer IO360™ Panel to evaluate predefined gene expression signatures (Table 6). In second-line axi-cel-treated patients, the B-cell signature (IO360™) was the only predefined signature associated (P<0.05) with higher probability of ongoing response (versus progression after response and no response; and improved EFS and duration of response (DOR; P<0.05; patients with high signature value [>median] versus those with low value [≤median]. The prespecified B-cell lineage signature included BLK, CD19, MS4A1, TNFRSF17, FCRL2, FAM30A, PNOC, SPIB, and TCL1A. Of those genes, expression of CD19, MS4A1, and TNFRSF17 was significantly (descriptive P<0.05) elevated in patients who were in ongoing response, with fold increase of 22%, 40%, and 69%, respectively. Conversely, expression of hypoxia, nitric oxide synthase 2 (NOS2), and the NKCD56dim NanoString signature (composed of IL21R, KIR2DL3, KIR3DL1, and KIR3DL2) were associated (P<0.05) with shorter EFS and/or DOR.

In the SOC arm, the B-cell signature was not associated with efficacy, and few NanoString signatures associated with efficacy endpoints. Immune gene expression signatures for macrophages, myeloid, antigen presentation machinery (APM), natural killer (NK), or CD8 T cells were marginally associated with either ongoing response, EFS, and/or DOR (none of the signatures consistently associated with all 3 efficacy metrics), suggesting that enrichment of select tumor immune infiltrates might be a factor supporting SOC responses. Nonetheless, axi-cel EFS was improved versus SOC for all subgroups, including high APM (>median).

TABLE 6
NanoString IO360 ™ panel gene signatures

| | Cell type signature, | |
|---|---|---|
| | biological activity signature, | |
| Cluster | or single gene signature | Gene name |
| 1: B-Cell Lineage | APM loss | B2M, TAP1, TAP2, TAPBP, HLA-A, |
| and Proliferation | | HLA-B, HLA-C |
| Index (BPI) | B cells | BLK, CD19, MS4A1, TNFRSF17, |
| | | FCRL2, FAM30A, PNOC, SPIB, |
| | | TCL1A |
| | CD45 | PTPRC |
| | Glycolytic activity | AKT1, HIF1A, SLC2A1, HK2, TPI1, |
| | | ENO1, LDHA, PFKFB3, PFKM, |
| | | GOT1, GOT2, GLUD1, HK1 |
| | JAK-STAT loss | JAK1, JAK2, IFNGR1, IRF1, |
| | | IFNGR2 |
| | MSI predictor | MLH1, MSH2, MSH6, PMS2, |
| | | EPM2AIP1, RNLS, SFXN1, SMAP1, |
| | | SREBF1, TTC30A, TYMS, WDR76, |
| | | WNT11, EIF5AL1 |
| | Proliferation | MKI67, CEP55, KIF2C, MELK, |
| | | CENPF, EXO1, ANLN, RRM2, |
| | | UBE2C, CCNB1, CDC20 |
| 2: Stromal and | Apoptosis | AXIN1, BAD, BAX, BBC3, BCL2L1 |
| Immunosuppressive | Endothelial cells | FAM124B, KDR, CLEC14A, CXorf36, |
| Index (SII) | | ROBO4, MYCT1, CDH5, TIE1, |
| | | BCL6B, PALMD, MMRN2 |
| | Hypoxia | BNIP3L, MXI1, ADM, PLOD2, |
| | | P4HA1, ALDOC, SLC2A1, PDK1, |
| | | P4HA2, BNIP3 |
| | MAGEs | MAGEA3/A6, MAGEA1, MAGEA12, |
| | | MAGEA4, MAGEB2, MAGEC2, |
| | | MAGEC1 |
| | Mast cells | MS4A2, CPA3, HDC, TPSAB1/B2 |
| | MMR loss | MLH1, PMS2, MSH2, MSH6 |
| | Myeloid inflammatory | CXCL1, CXCL3, CXCL2, CCL20, |
| | | AREG, FOSL1, CSF3, PTGS2, IER3, |
| | | IL6 |
| | Stroma | FAP, COL6A3, ADAM12, OLFML2B, |
| | | PDGFRB, LRRC32 |
| | Single gene signatures | ARG1, CD276 (B7-H3), NOS2, TGFB1 |
| 3 | Dendritic cells | CCL13, CD209, HSD11B1 |
| | IFN downstream | IFI16, IFI27, IFI35, IFIH1, IFIT1, |
| | | IFIT2, IFITM1, IFITM2, IRF1, |
| | | APOL6, TMEM140, PARP9, TRIM21, |
| | | GBP1, DTX3L, PSMB9, OAS1, OAS2, |
| | | ISG15, MX1, IFI6, IFIT3, IRF9, |
| | | STAT2 |
| | IFN-γ | STAT1, CXCL9, CXCL10, CXCL11 |
| | Immunoproteasome | PSMB8, PSMB9, PSMB10 |
| | Inflammatory chemokines | CCL2, CCL3/L1, CCL4, CCL7, CCL8 |
| | Macrophages | CD163, CD68, CD84, MS4A4A |
| | MHC2 | HLA-DRB5, HLA-DPA1, HLA-DPB1, |
| | | HLA-DQB1, HLA-DRA, HLA-DRB1, |
| | | HLA-DMA, HLA-DOA |
| | Myeloid | ITGAM, TLR4, IL1B, CSF1R, CSF3R, |
| | | TLR2, TLR1, ITGAX, HCK, TLR8, |
| | | SLC11A1, CD47, CD14, CLEC4E, |
| | | CLEC7A, FCAR, FCN1, LILRA5, |
| | | LILRB2, LYZ, NFAM1, P2RY13, |
| | | S100A8, S100A9, SERPINA1, SIRPA, |
| | | SIRPB2, TREM1, CLEC5A, CSF1, |
| | | CYBB, FCGR1A, MARCO, NLRP3, |
| | | FPR1, FPR3, CCL3/L1, DAB2, OLR1, |
| | | C5AR1, TREM2, MRC1, CEBPB |
| | Neutrophils | CSF3R, S100A12, CEACAM3, FCAR, |
| | | FCGR3A/B, FPR1, SIGLEC5 |
| | NK cells | NCR1, XCL1/2 |
| | NK CD56dim cells | IL21R, KIR2DL3, KIR3DL1, KIR3DL2 |
| | Th1 cells | TBX21 |
| | Treg | FOXP3 |
| | Single gene signatures | IL10, CD274 (PD-L1), TIGIT |
| 4 | APM | B2M, TAP1, TAP2, TAPBP, HLA-A, |
| | | HLA-B, HLA-C |
| | CD8 T cells | CD8A, CD8B |
| | Cytotoxic cells | CTSW, GNLY, GZMA, GZMB, GZMH, |
| | | KLRB1, KLRD1, KLRK1, PRF1, |
| | | NKG7 |
| | Cytotoxicity | GZMA, GZMB, GZMH, PRF1, GNLY |
| | Exhausted CD8 | CD244, EOMES, LAG3, PTGER4 |
| | Lymphoid | CXCL10, CXCR3, CX3CL1, PRF1, |
| | | GZMK, GZMB, CD27, IL2RG, |
| | | KLRK1, CTLA4, GZMH, CD3D, |
| | | KLRB1, KLRD1, LCK, CD5, IRF4, |
| | | CD8A, CD38, EOMES, GZMM, |
| | | GNLY, IFITM1, IDO1, MS4A1, |
| | | GZMA, CD2, CD3E, CD3G, CD40LG, |
| | | CD6, CD7, CD79A, CD8B, CXCL11, |
| | | CXCL13, CXCL9, HLA-DOB, IFNG, |
| | | LAG3, LY9, PDCD1, TBX21, TIGIT, |
| | | ZAP70, SLAMF7, CD96, PVR, STAT1, |
| | | JAK1, JAK2, STAT2, IRF9, IGF2R, |
| | | CD48, ICOS |
| | T cells | CD3D, CD3E, CD3G, CD6, SH2D1A, |
| | | TRAT1 |
| | TIS | CCL5, CD27, CD274, CD276, CD8A, |
| | | CMKLR1, CXCL9, CXCR6, HLA- |
| | | DQA1, HLA-DRB1, HLA-E, IDO1, |
| | | LAG3, NKG7, PDCD1LG2, PSMB10, |
| | | STAT1, TIGIT |
| | Single gene signatures | CTLA4, IDO1, PDCD1LG2 (PD-L2), |
| | | PDCD1 (PD-1) |

In the above table, genes that are negatively associated with the cluster in which they are found are shown in bold. All other genes demonstrated a positive association with the cluster. APM, antigen processing machinery; IFN, interferon; JAK, Janus kinase; MAGE, melanoma antigen gene; MHC, major histocompatibility complex; MMR, mismatch repair; MSI, microsatellite instability; NK, natural killer; STAT, signal transducer and activator of transcription; Th1, T helper type 1; TIS, tumor inflammation signature; Treg, regulatory T cells.

Clustering Revealed Distinct Gene Expression Signatures, Consistent with Different TME Immune Contextures

Based on unsupervised clustering analyses of NanoString IO360™ results, 4 major clusters of gene expression signatures were identified, underlying different tumor microenvironment (TME) immune contextures (Table 6). The signatures within each cluster were combined to create indices for further analysis. The first cluster, herein referred to as the B-Cell Lineage and Proliferation Index (BPI), included signatures like B-cell, proliferation, APM loss, and glycolytic activity. Signatures from BPI presented the highest hierarchically separation from the other 3 major clusters, suggesting a relatively simpler TME with abundant and highly proliferative cancer cells and lower immune cell infiltration versus the other clusters. A second cluster, termed the Stromal and Immunosuppressive Index (SII), featured gene sets inclusive of stroma, myeloid and endothelial cells, NOS2, transforming growth factor 3 (TGF-0), B7-H3, arginase 1 (ARG1), and hypoxia. In this cluster, hypoxia and NOS2 signatures (IO360) were negatively associated with EFS and/or DOR following axi-cel treatment. A third cluster was enriched for signatures of NK cells, macrophages, and antigen-presenting cells. A fourth cluster primarily consisted of T-cell infiltration features. The third and fourth clusters showed a relatively close hierarchical correlation, perhaps jointly representing tumors that are more complex and immune-infiltrated.

SII and BPI (TME contextures) associated with EFS in the axi-cel arm

Root mean square indices were generated (see methods) from each of the above 4 clusters of NanoString signatures. BPI (cluster 1) and SII (cluster 2) were positively and negatively, respectively, associated with EFS in the axi-cel arm (descriptive P<0.05). None of the 4 clusters associated with cell of origin. Notably, BPI positively associated with high-grade B-cell lymphoma (HGBL) and double-/triple-hit disease. Median EFS in the HGBL subgroup following axi-cel treatment was 21.5 months (95% CI, 3.7—not evaluable; unstratified HR [axi-cel over SOC]=0.318). While EFS of the HGBL subtype was not significantly different from that of the non-HGBL subtype (DLBCL+others), a directionally favorable EFS was seen in HGBL versus non-HGBL subtype in the axi-cel arm (unstratified HR [HGBL over non-HGBL]=0.692; 95% CI, 0.384-1.245), suggesting that patients with HGBL might benefit more from axi-cel relative to others. Conversely, the SOC arm showed the opposite, non-significant directional association (unstratified HR [HGBL over non-HGBL]=1.17; 95% CI, 0.723-1.892). The third and fourth clusters were not associated with EFS and/or DOR following axi-cel treatment. In contrast, none of these four clusters were significantly associated with outcome in the SOC arm, which indicates the differential influence of TME on outcomes with SOC and CAR T-cell therapy.

### CD19 Expression Level had Differential Impact on Efficacy Based on TME and Therapy

CD19 protein level (H-score) was correlated with CD19 gene expression and the B-cell signature. Consistent with a role for the B-cell signature in axi-cel-mediated efficacy, CD19 gene expression and protein level also correlated with axi-cel EFS. Axi-cel EFS was better in patients with high (>median) CD19 gene and protein expression levels relative to those with lower expression (≤median). Notably, axi-cel remained superior to SOC across CD19 gene expression subgroups. Patients deemed CD19 negative by immunohistochemistry (H-score≤5) still presented substantial responses to axi-cel, with 85% objective response rate (ORR) versus 67% ORR in the SOC arm.

Patients with lower CD19 protein expression (H-score≤median) harbored a more complex, immune-infiltrated TME enriched with multiple immunosuppressive features, including gene expression signatures for regulatory T cells, T-cell exhaustion, ARG1, indoleamine 2,3-dioxygenase 1 (IDO1), B7-H3, CTLA4, and macrophage and myeloid cells. The poorest EFS after axi-cel treatment was observed in patients with tumors that harbored both low CD19 protein expression and high SII, suggesting that both TME immunosuppression and target expression have a role in resistance to CAR T-cell therapy. Conversely, in patients with higher CD19 protein expression (>median), lack of durable response was associated with glycolytic activity.

### Axi-Cel Product Enriched in CCR7+CD45RA+ T Cells May Overcome Low Antigen (CD19) Expression

It was further investigated whether an axi-cel product enriched in CCR7+CD45RA+ T cells, considered a naive-like T cell or stem memory phenotype (T_{SCM}), may overcome the adverse effects of an unfavorable TME. Indeed, patients with relatively lower CD19 expression showed improved EFS when there was a higher frequency of CCR7+CD45RA+ T cells in the product. Similar results were obtained with the total number of CCR7+CD45RA+ T cells infused. Patients with relatively higher SII also showed improved EFS with higher frequency of CCR7+CD45RA+ T cells in the product, but the differential was not significant by descriptive statistics.

ZUMA-7 is the largest available clinical dataset in the setting of CAR T-cell therapy for second-line LBCL. Here, the ZUMA-7 dataset was explored to uncover novel tumor biomarkers associated with response (EFS, DOR, ongoing response, CR, objective response) to CAR T-cell therapy (axi-cel) or SOC (HDT-ASCT). It was determined that outcomes to axi-cel or SOC are differentially influenced by the composition of the TME in the second-line setting, providing insights into the putative mechanisms driving responsiveness to these therapies. For instance, tumor gene expression signatures, including B-cell score and a cluster enriched with stromal and immune-suppressive features (SII), were positively and negatively associated, respectively, with CAR T-cell therapy outcome. Notably, within the B-cell score, CD19 gene and protein expression were also positively associated with cell therapy outcome.

The analyses reported herein identified major clusters of gene expression signatures. The SII (cluster 2 in Table 6), which negatively associated with clinical outcome, is likely reflective of an immune-suppressive TME, including myeloid-associated immune suppressive and TGF-β-activated stromal genes. Within this immune contexture, CAR T cells may not sufficiently traffic to malignant cells or sustain a functional state. In contrast, and unexpectedly, the BPI (cluster 1 in Table 6), was positively associated with HGBL/double-/triple-hit disease and a favorable clinical outcome. This high-risk subgroup was more likely to have high BPI indices, indicating a more uniform malignant B-cell population with less diverse immune infiltration.

Another finding of this study was the clear distinction between axi-cel and SOC biomarkers associated with outcome. Interestingly, while the prespecified B-cell lineage signature and CD19 expression associated positively with outcomes after axi-cel treatment, other TME immune features, including APM and DC, associated positively with outcomes after SOC. This suggests a mechanistic distinction between direct antigen engagement of CD19 by the CAR under axi-cel, versus co-opting the endogenous immunity against tumor epitopes under SOC. Notably, outcomes with axi-cel were improved versus those with SOC for all presented biomarker subgroups.

Collectively, these findings uncovered novel gene expression signatures that carry potential predictive value to responses with axi-cel. These findings also help guide therapeutic decisions, including whether a patient would benefit from a cellular therapy as a second-line over a standard of care. The four clusters of TME signatures described herein and the associations of clusters 1 (BPI) and 2 (SII) with CAR T-cell therapy outcomes have not been previously reported.

Here, is presented a strong association between cluster 1 (B-cell proliferation) and HGBL. It is also reported that the B-cell proliferation and stromal clusters are associated with best and worst outcome, respectively. These observations might have important implications with CAR T-cell therapy and other therapeutics moving to earlier lines of treatment, as there may be response-predictive value to these TME signatures.

A previously underappreciated correlation of baseline CD19 expression with TME immune infiltration and with axi-cel outcomes is reported herein. An association was found between CD19 expression and outcome by IHC or gene expression profiling with NanoString. Axi-cel demonstrated improved EFS over SOC regardless of CD19 protein expression protein/H-score. Nevertheless, in the ZUMA-7 axi-cel arm, patients with lower CD19 protein expression presented a more complex, immune-infiltrated TME, underscoring that the relatively shorter EFS of axi-cel in patients with relatively lower CD19 protein expression (H-score≤median) may not only be dependent on suboptimal target expression, but also on concurrent and confounding immune contexture features. In fact, low CD19 H-score associated with cluster 2 (SII), which negatively associated with axi-cel EFS, and the association between CD19 H-score and EFS appeared confined to patients having a high SII index.

While patients with reduced B-cell signature and less favorable tumor immune microenvironment showed a poorer clinical outlook, a key question is whether actionable product characteristics may help overcome such unfavorable features. As presented herein, a CAR T-cell product enriched in the CCR7+CD45RA+ T-cell phenotype may improve outcomes in patients with lower CD19 protein expression and higher immunosuppressive features.

Knowledge of the immune contexture is essential for understanding mechanisms of action and likelihood of prolonged response to CAR T-cell therapy. In addition to SPD, MTV, LDH, and target (CD19) expression, measurements of tumor immune contexture using Immunoscore, IS21, B cell, as well as stromal and immunosuppressive gene signatures, are emerging as important and interrelated determinants of durable responses to axi-cel intervention. Earlier intervention with CAR T-cell therapy is supported by a more conducive immune contexture for increased axi-cel activity, which may have contributed to the superior efficacy of axi-cel compared with SOC in second-line LBCL across common prognostic subgroups. Furthermore, patients with HGBL/double-/triple-hit disease, a feature associated with worse outcomes to conventional chemoimmunotherapy, substantially benefited from axi-cel treatment. The enrichment of BPI and, thus, the higher prevalence of proliferative B cells, CD19 expression, and relatively fewer immunosuppressive cells in HGBL/double-/triple-hit tumors may underscore the sensitivity of this high-risk population to CD19-directed CAR T-cell therapy in comparison with SOC.

### Patient Samples and Efficacy Readouts

Evaluable samples from patients in the safety analysis sets of ZUMA-7 (N=170) and ZUMA-1 Cohorts 1+2 (N=101) were analyzed. The safety analysis set of ZUMA-7 was defined as randomized patients who received at least one dose of axi-cel or SOC. The safety analysis set of ZUMA-1 was defined as all patients treated with any dose of axi-cel. The studies were approved by the institutional review board at each study site and were conducted in accordance with the Good Clinical Practice guidelines of the International Conference on Harmonization.

ZUMA-7 efficacy endpoints (ORR, best response, EFS, DOR, and ongoing response) utilized the primary analysis data cutoff date. EFS was defined as time from randomization to the earliest date of disease progression per Lugano Classification, commencement of new lymphoma therapy, or death from any cause. Ongoing response was defined as patients who were in ongoing response (CR or partial response (PR)) by the ZUMA-7 primary analysis data cutoff date. Progression after response was defined as patients who achieved a CR or PR and subsequently experienced disease progression. Patients who achieved stable disease or progressive disease as best response were included within the category of no response, as previously reported. To contextualize select findings, data from patients with evaluable samples in ZUMA-1 pivotal Cohorts 1+2 were included with a minimum follow-up of 60 months.

### Analysis of Gene Expression Signatures

Tumor biopsy collection and processing of formalin-fixed paraffin-embedded biopsy specimens was similar between ZUMA-1 and ZUMA-7. Gene expression and molecular subgroup analysis were performed by leveraging the NanoString PanCancer IO360™ Panel and Lymphoma Subtyping Test. Predefined gene expression signatures from NanoString (proprietary algorithm) were analyzed for clustering and association with efficacy readouts. Unsupervised clustering of gene expression signatures was performed in TIBCO Spotfire using the calculated hierarchical clustering method (Unweighted Pair Group Method with Arithmetic Mean [UPGMA]; distance measure-Euclidean, ordering weight-average value, empty value replacement method-constant value, replace with 0 and normalization-none). IS21, a predefined gene expression signature of T-cell infiltration and function, was calculated from gene expression values of the PanCancer IO360™ Panel using a proprietary algorithm from Veracyte. Based on the PanCancer IO360™ Panel, cell subtypes within the TME and their association with clinical outcome were investigated.

### Analysis of CD19 Expression Level

CD19 protein expression level was measured by IHC using a validated assay at NeoGenomics.

### Analysis of Product Attributes

Product T-cell phenotypes and other product attributes were assessed as previously described. Additional product characterization of costimulatory (CD27, CD28) and activation and exhaustion markers (programmed cell death protein 1, T-cell immunoglobulin and mucin domain-containing protein 3, lymphocyte activation gene 3) was performed by flow cytometry using a validated assay at CellCarta.

### Association Analysis and Related Statistics

Biomarkers from exploratory endpoints were analyzed for associations with each other and with efficacy endpoints. Spearman's rank-order correlation was used to evaluate association between analytes. Kaplan-Meir plots and Cox regression were used to evaluate association between biomarkers and time-to-event endpoints. Wilcoxon rank sum test and logistic regression were used to evaluate the relationship between biomarkers and binary outcomes. Kruskal-Wallis tests were used to evaluate association between biomarkers and categorical endpoints. For these post hoc analyses, all P values were descriptive and P<0.05 was considered significant. No adjustments for multiplicity testing were performed. Covariates were subdivided into subgroups by median value, quartile values, or as indicated (eg, SPD value of 3721 mm²). Plots were generated using TIBCO Spotfire, SAS, R, or GraphPad Prism.

### Example 2

This example shows that in patients with early relapsed or primary refractory large B-cell lymphoma (R/R LBCL), axicabtagene ciloleucel (axi-cel; autologous anti-CD19 chimeric antigen receptor T-cell therapy) as second-line therapy significantly improved event-free survival over the standard-care arm in the international phase 3 ZUMA-7 trial.

Patients were randomized 1:1 to axi-cel or standard care (2-3 cycles of protocol-defined chemoimmunotherapy followed in responding patients by high-dose chemotherapy with autologous stem-cell transplantation [HDT-ASCT]). The intention-to-treat primary analysis of overall survival occurred per protocol 5 years after the first patient was randomized.

In total, 359 patients were randomized to axi-cel (n=180) or standard care (n=179). At 47.2-month median follow-up, axi-cel demonstrated a statistically significant improvement in overall survival over standard care (hazard ratio, 0.726; 95% CI, 0.540-0.977; stratified 1-sided log-rank P=0.0168). Median overall survival was not reached in the axi-cel arm and was 31.1 months in the standard-care arm; estimated 48-month overall survival rates were 54.6% and 46.0%, respectively. The survival benefit favoring axi-cel was consistent across prespecified key subgroups, including age≥65 years, primary refractory, or high-grade B-cell lymphoma. Median investigator-assessed progression-free survival was 14.7 months for axi-cel versus 3.7 months for standard care, with estimated 48-month rates of 41.8% versus 24.4%, respectively (hazard ratio, 0.506; 95% CI, 0.383-0.669). Axi-cel as second-line treatment for R/R LBCL significantly improved overall survival compared with standard care.

For nearly 30 years, the standard-care paradigm for second-line treatment of large B-cell lymphoma (LBCL) with curative intent has been multistep platinum-based chemotherapy followed in responding patients by high-dose chemotherapy and autologous stem-cell transplantation (HDT-ASCT). However, only half of all patients are likely to be eligible for this approach, of whom approximately 20% are ultimately cured. Outcomes for patients who cannot proceed to HDT-ASCT are poor, with a median overall survival (OS) of 4.4 months.

Given this unmet need and the approval of chimeric antigen receptor (CAR) T-cell therapy in third line or later settings, the phase 3 ZUMA-7 trial (NCT03391466) was designed to compare axicabtagene ciloleucel (axi-cel), an anti-CD19 autologous CAR T-cell therapy given as a one-time dose, with a second-line standard-care therapy in patients with early relapsed or primary refractory (R/R) LBCL. The primary endpoint of event-free survival (EFS) by blinded central review demonstrated that axi-cel was superior to standard care (hazard ratio [HR], 0.398; stratified log-rank P<0.0001). With a median follow-up of 24.9 months, median EFS was 8.3 versus 2.0 months and 24-month EFS rates were 41% versus 16% in the axi-cel versus standard care arms, respectively. A response by blinded central review occurred in 83% of patients in the axi-cel arm and 50% in the standard-care arm (with a complete response in 65% and 32%, respectively). We now report the primary OS analysis of ZUMA-7 at the protocol-defined timepoint of 5 years after the first patient was randomized.

### Methods

### Trial Design and Oversight

Adult patients (≥18 years) had histologically confirmed LBCL which was refractory to first-line treatment or had relapsed from complete response within 12 months of first-line chemoimmunotherapy.

Patients were randomized 1:1 to axi-cel or standard care (2-3 cycles of investigator-selected, protocol-defined chemoimmunotherapy followed by, in patients with a complete or partial response, HDT-ASCT). Randomization was stratified by response to first-line therapy and second-line age-adjusted International Prognostic Index (aaIPI). Although crossover between treatment arms was not planned per protocol, patients could receive subsequent therapy off-protocol, including cellular immunotherapy after standard care (defined as treatment switching).

### Endpoints and Assessments

The primary endpoint of ZUMA-7 was EFS (time from randomization to progression, death, or new lymphoma therapy) per blinded central review. Protocol-defined key secondary endpoints included objective response rate per blinded central review and OS (time from randomization to death from any cause). Secondary endpoints included progression-free survival (PFS; time from randomization to disease progression or death from any cause) and EFS, reported herein per investigator assessment as blinded central review was discontinued per protocol after the primary EFS analysis. Disease assessments occurred on days 50, 100, and 150 after randomization, then every 3 months until 2 years and every 6 months until 5 years of follow-up.

### Statistical Analysis

The prespecified intention-to-treat (ITT) primary OS analysis was to be conducted after 210 deaths or no later than 5 years after the first patient was randomized and was triggered by the latter criterion. A group sequential testing procedure for OS was performed to control the overall 1-sided alpha of 2.5%. A log-rank test stratified by randomization stratification factors was used for the primary comparison of OS with an efficacy boundary with 1-sided significance level of 0.0249. In addition to the ITT analysis, and to adjust for the confounding effect of treatment switching in the standard-care arm to off-protocol cellular immunotherapy, two prespecified OS sensitivity analyses were conducted.

Efficacy analyses based on the ITT principle included all randomized patients. Safety analyses included all randomized patients who received ≥1 dose of axi-cel or standard care per protocol. All AEs were reported from randomization through the Day 150 post-randomization visit or change in lymphoma therapy, whichever came first. After Day 150, targeted serious AEs were reported through the data cutoff date, until disease progression, or initiation of new lymphoma therapy, whichever occurred first. Serious AEs that the investigator assessed as related to axi-cel were reported regardless of time of occurrence.

### Further Methods

### Study Treatment

Patients in the axicabtagene ciloleucel (axi-cel) arm underwent leukapheresis, followed by lymphodepleting chemotherapy with cyclophosphamide (500 mg/m²/day) and fludarabine (30 mg/m²/day) 5, 4, and 3 days before receiving a single axi-cel infusion (target dose, 2×10⁶ chimeric antigen receptor [CAR] T cells/kg). Glucocorticoid could be administered as optional bridging therapy. Patients in the standard-care arm received 2 or 3 cycles of protocol-defined, platinum-based chemoimmunotherapy. Patients who had a complete or partial response proceeded to high-dose chemotherapy with autologous stem-cell transplantation.

### Endpoints and Assessments

Event-free survival (EFS) was defined as the time from randomization to the earliest date of disease progression according to the Lugano classification, the commencement of new therapy for lymphoma, death from any cause, or a best response of stable disease up to and including the response on the day 150 assessment after randomization. Safety outcomes included incidence of adverse events (AEs). Guidelines for the management of CAR T-cell-related AEs followed those used in ZUMA-1. AEs, including symptoms related to cytokine release syndrome (CRS) and neurologic events, were graded according to the National Cancer Institute Common Terminology Criteria for AEs, version 4.03. CRS severity was graded per modified Lee criteria.

### Reporting of Serious Adverse Events

Targeted serious adverse events were defined as and included neurological, hematological, infections, autoimmune disorders, and secondary malignancies and were reported for up to 5 or 15 years for the standard-care or axi-cel arms, respectively, or until disease progression, whichever occurred first. Serious adverse events that the investigator assessed as related to axi-cel were reported regardless of the time period.

### Analysis of Overall Survival

Per group sequential testing procedure with pre-specified rho-family spending function to control the overall 1-sided alpha of 2.5%, overall survival (OS) could be tested up to 3 times. Per protocol, the first interim analysis of OS occurred at the time of the primary EFS analysis. The second interim analysis of OS was to occur when approximately 160 deaths were observed or not later than 4 years after the first patient was randomized. At the time of the EFS primary analysis, approximately 160 deaths were observed. For this reason, the interim OS analysis conducted at the time of the primary EFS analysis was the only interim analysis, meeting criteria for both originally planned interim OS analyses. The only subsequent planned OS analysis was the primary OS analysis, reported herein, which was planned to occur when 210 death events were observed or no later than 5 years after the first patient was randomized, and which did occur 5 years after the first patient was randomized (as less than 210 deaths had occurred at that protocol-defined timepoint).

### Exploratory Analyses

Exploratory analyses were conducted to determine the association between OS and axi-cel pharmacokinetics and product features. Anti-CD19 CAR T-cell levels in blood were quantified and product T-cell phenotypes and other attributes were assessed as previously described. Variables were characterized using the median value (i.e., ≤median and >median values) as the cutoff point for comparisons.

The presence of B cells in blood was evaluated through 24 months in the axi-cel arm using flow cytometry as previously described. B-cell aplasia was defined as undetectable B-cell levels (i.e., below the lower limit of quantitation of the assay used [<0.017% of leukocytes]). B-cell recovery was defined as detectable B-cell levels compared with prior timepoints.

Prolonged cytopenias were evaluated post hoc over 4 time intervals and were defined as those present on or after 6 months, 12 months, 18 months, and 24 months from initiation of definitive therapy on protocol (i.e., from receipt of axi-cel infusion or first dose of high-dose therapy).

### Additional Statistical Methods

As previously reported, Kaplan-Meier estimates were provided for time-to-event endpoints. Estimated hazard ratios with two-sided 95% confidence intervals were calculated from a Cox proportional-hazards model stratified by the randomization stratification factors. Stratified log-rank P values (one-sided) were calculated for time-to-event endpoints.

To adjust for the confounding effect of treatment switching in the standard-care arm to off-protocol cellular immunotherapy, prespecified OS sensitivity analyses were conducted using two validated methods: Rank Preserving Structural Failure Time (RPSFT) Model with g-estimation, and Inverse Probability of Censoring Weights (IPCW). Patients in the standard-care arm who received cellular immunotherapy were not censored at time of cellular immunotherapy. In the model, the survival/death time for standard-care patients after cellular immunotherapy was reduced as if cellular immunotherapy had not been given.

Associations between product characteristics or levels of CAR T cells and efficacy endpoints were explored by post hoc univariate analysis, with descriptive P values reported. There was no adjustment for multiplicity testing. Covariates were continuous or categorized using median values. Hazard ratios were calculated by the Cox regression model as percentage increase in hazards for one-unit increase of continuous variables. Stratified (derived) Cox proportional hazards P values were calculated.

### Results

### Patients

Between Jan. 25, 2018, and Oct. 4, 2019, 359 patients were enrolled and randomized to axi-cel (n=180) or standard care (n=179). Patient demographics and disease characteristics at baseline were similar between treatment arms, and consistent with real-world patient populations receiving CAR T-cell therapy (Table 7). High risk features were common, including 19% with high-grade B-cell lymphoma (HGBL; including double-hit lymphomas) per investigator assessment, 45% with high second-line aaIPI score (2 or 3), 54% with elevated lactate dehydrogenase level, and 74% with disease refractory to first-line therapy.

TABLE 7
Representativeness of the Zuma-7 Study Participants

| Category | |
|---|---|
| Special considerations | |
| related to | Key Findings |
| Age | LBCL predominantly affects older adults. Irrespective of race or |
| | sex, LBCL diagnoses are made at increasing frequency with |
| | increasing age. The patient population seeking second-line |
| | treatment observed in real-world evidence is, on average, older |
| | than those participating in clinical trials. |
| Race or ethnic group | The majority of patients with LBCL treated in the second line |
| | are non-Hispanic whites, though the age-adjusted incidence rates |
| | of LBCL among non-Hispanic whites and Hispanics are similar, |
| | followed by smaller proportions of other race or ethnic groups, |
| | including non-Hispanic blacks. |
| Sex and gender | Overall, LBCL proportionately affects more men than women. |
| | This prevalence is exaggerated in the second-line clinical trials |
| | assessed compared with that observed in real-world evidence. |
| | Generally, slightly more men than women are diagnosed with |
| | LBCL, though this varies across race and ethnic groups. |
| Overall | The demographics of the participants in the ZUMA-7 study were |
| representativeness of | consistent with those expected of this relapsed/refractory patient |
| this trial | population with LBCL treated in clinical trials. The median |
| | patient age, the ratio of men to women, and the proportion of |
| | non-Hispanic white patients was consistent with that observed in |
| | clinical trials in this setting. However, a greater proportion of |
| | patients aged ≥65 years were enrolled in ZUMA-7 compared |
| | with most of the clinical studies analyzed. |
| | Generally, the patient demographics in ZUMA-7, along with the |
| | clinical studies analyzed here, demonstrate the |
| | underrepresentation of racial minorities in cancer trials. For |
| | example, a greater proportion of non-Hispanic whites were |
| | enrolled and treated in ZUMA-7 compared with other racial and |
| | ethnic groups. However, while a numerically slightly lower |
| | proportion of non-Hispanic blacks in the United States were |
| | enrolled and treated in ZUMA-7 versus simulated racial |
| | disposition based on disease prevalence in the US and locations |
| | of CAR T-cell therapy authorized treatment centers, there was |
| | no statistically significant difference. Still, additional inclusive |
| | research is needed to address known inequalities in medical |
| | practice and complete datasets that include full patient |
| | demographic information. |

### Efficacy

At a median follow-up of 47.2 months (range, 39.8-60.0), 82 patients in the axi-cel arm and 95 patients in the standard-care arm had died. The primary analysis of OS demonstrated a statistically significant improvement in OS with axi cel over standard care (HR for death, 0.726; 95% CI, 0.540-0.977; stratified 1-sided log-rank P=0.0168). Median 05 (95% CI) was not reached (28.6 months—not estimable [NE]) with axi-cel and was 31.1 months (17.1-NE) with standard care (Table 8). The estimated OS rate at 48 months was 54.6% (95% CC, 47.0-61.6) with axi-cel and 46.0% (38.4-53.2) with standard care (Table 9). The OS benefit of axi cel over standardcare was consistent across key prespecified high-risk subgroups, including age≥65 years, primary refractory disease, high second-line aaIPI, and HGBL (including double-hit lymphoma) (Table 10).

TABLE 8
Overall Survival. Kaplan-Meier estimate of overall survival. Patients
who did not meet the criteria for an event had their data censored.
One-sided P value from the log-rank test is reported.

| | Standard of | |
|---|---|---|
| Axi-Cel (n = 180) | Care (n = 179) | |
| NR (28.6-NE) | 31.1 (17.1-NE) | Median OC (95%, CI), mo |
| 0.726 (.540-0.977) | | Stratified HR (95% CI) |
| 0.168 | | Stratified P = Value |
| 76 | 63 | 1-year OS Estimate (%) |
| 60 | 51 | 2-year OS Estimate (%) |
| 56 | 48 | 3-year OS Estimate (%) |
| 55 | 46 | 4-year OS Estimate (%) |

Axi-cel, axicabtagene ciloleucel; HR, hazard ratio; NE, not estimable; NR, not reached; OS, overall survival.

TABLE 9
Kaplan-Meier Estimates of Overall Survival
in Axi-Cel and Standard-Care Arms

| | Axi-Cel | Standard Care |
|---|---|---|
| % (95% CI) | N = 180 | N = 179 |
| 3-month OS | 96.7 (92.7-98.5) | 97.7 (94.1-99.1) |
| 6-month OS | 90.0 (84.6-93.6) | 85.2 (79.0-89.6) |
| 9-month OS | 83.9 (77.6-88.5) | 72.6 (65.3-78.6) |
| 12-month OS | 76.0 (69.1-81.6) | 63.4 (55.8-70.1) |
| 15-month OS | 67.6 (60.3-74.0) | 58.3 (50.6-65.2) |
| 18-month OS | 64.8 (57.4-71.3) | 56.6 (48.9-63.5) |
| 21-month OS | 62.0 (54.5-68.7) | 52.0 (44.3-59.1) |
| 24-month OS | 60.4 (52.8-67.1) | 51.4 (43.7-58.5) |
| 27-month OS | 58.7 (51.1-65.5) | 50.8 (43.2-57.9) |
| 30-month OS | 55.9 (48.3-62.8) | 50.2 (42.6-57.3) |
| 33-month OS | 55.9 (48.3-62.8) | 49.0 (41.4-56.2) |
| 36-month OS | 55.9 (48.3-62.8) | 47.8 (40.2-55.0) |
| 39-month OS | 55.9 (48.3-62.8) | 46.0 (38.4-53.2) |
| 42-month OS | 55.3 (47.7-62.3) | 46.0 (38.4-53.2) |
| 45-month OS | 54.6 (47.0-61.6) | 46.0 (38.4-53.2) |
| 48-month OS | 54.6 (47.0-61.6) | 46.0 (38.4-53.2) |

TABLE 10
Overall Survival for Prespecified Subgroups. Subgroup analysis of overall survival
for key baseline and clinical covariates, including response to first-line
therapy and second-line age-adjusted IPI at randomization. Stratified Cox regression
models were used to provide the estimated hazard ratios and 2-sided 95% confidence
intervals for axi-cel relative to standard care hazard ratio. The Breslow method
was used to handle the ties for the Cox regression models.

| | No. of patients with | | | | |
|---|---|---|---|---|---|
| | Events/Total No. | | | | |
| | Axi-cel No. | Standard | Hazard | 95% CI | |
| | of Patients | care | Ratio | LCI | UCI |
| Overall | 82/180 | 95/179 | 0.726 | 0.540 | 0.977 |

Age

| | | | | | |
|---|---|---|---|---|---|
| <65 | 56/129 | 64/121 | 0.779 | 0.541 | 1.120 |
| ≥65 | 26/51 | 31/58 | 0.691 | 0.401 | 1.190 |

Geographic Region

| | | | | | |
|---|---|---|---|---|---|
| US | 59/130 | 61/120 | 0.758 | 0.529 | 1.086 |
| Other | 23/50 | 34/59 | 0.690 | 0.405 | 1.177 |

Response to 1^{st}-line therapy at randomization

| | | | | | |
|---|---|---|---|---|---|
| Primary refractory disease | 66/133 | 72/131 | 0.773 | 0.553 | 1.080 |
| Relapse ≤12 mo. | 16/47 | 23/48 | 0.586 | 0.308 | 1.116 |

2^{nd}-line age-adjusted IPI

| | | | | | |
|---|---|---|---|---|---|
| 0 or 1 | 37/98 | 40/100 | 0.842 | 0.538 | 1.317 |
| 2 or 3 | 45/82 | 55/79 | 0.647 | 0.436 | 0.962 |

Prognostic Marker according to central lab

| | | | | | |
|---|---|---|---|---|---|
| HGBL, double hit | 14/32 | 14/25 | 0.716 | 0.330 | 1.553 |
| Double expressor lymphoma | 26/57 | 33/62 | 0.729 | 0.435 | 1.221 |

Molecular subgroup

| | | | | | |
|---|---|---|---|---|---|
| Germinal center B-cell-like | 45/109 | 48/99 | 0.779 | 0.517 | 1.172 |
| Activated B-cell-like | 10/16 | 6/9 | 0.469 | 0.133 | 1.656 |
| Unclassified | 7/17 | 11/14 | 0.344 | 0.111 | 1.067 |

Disease type per investigator

| | | | | | |
|---|---|---|---|---|---|
| DLBCL | 51/110 | 62/116 | 0.728 | 0.501 | 1.059 |
| Large-cell transformation from FL | 9/19 | 15/27 | 0.728 | 0.315 | 1.685 |
| HGBL, including rearrangement of | 18/43 | 13/27 | 0.720 | 0.344 | 1.507 |
| MYC with BCL2 or BCL6 or both | | | | | |

Disease type percentral laboratory

| | | | | | |
|---|---|---|---|---|---|
| DLBCL | 60/126 | 65/120 | 0.761 | 0.534 | 1.083 |
| HGBL, including rearrangement of | 14/32 | 14/26 | 0.735 | 0.338 | 1.600 |
| MYC with BCL2 or BCL6 or both | | | | | |

Axi-cel, axicabtagene ciloleucel; BCL, B-cell lymphoma; DLBCL, diffuse large B-cell lymphoma; HGBL, high-grade B-cell lymphoma; IPI, International Prognostic Index; LCI, lower confidence interval; UCI, upper confidence interval.

In the standard-care arm, 102 (57%) patients received subsequent cellular immunotherapy off-protocol due to progression or lack of response (Table 11). Prespecified sensitivity analyses designed to assess the confounding effect of treatment switching on OS in the standard-care arm showed an even greater OS benefit with axi-cel versus standard care (Table 12).

TABLE 11
Subsequent Therapies Received by Class of Agent.

| | | |
|---|---|---|
| | Axi-Cel | Standard Care |
| n (%) | N = 180 | N = 179 |

| | | | | |
|---|---|---|---|---|
| Any | 88 | (49) | 128 | (72) |
| Cellular immunotherapy | 14 | (8) | 102 | (57) |
| Non-cellular | 35 | (19) | 32 | (18) |
| immunotherapy* | | | | |
| Chemoimmunotherapy | 73 | (41) | 77 | (43) |
| HDT + ASCT | 13 | (7) | 7 | (4) |
| HDT + alloSCT | 14 | (8) | 7 | (4) |
| Targeted therapy^{†} | 18 | (10) | 11 | (6) |
| Investigational product on | 3 | (2) | 2 | (1) |
| clinical study, not otherwise | | | | |
| specified | | | | |
| Steroids | 8 | (4) | 16 | (9) |
| Radiation | 16 | (9) | 28 | (16) |
| Surgery | 2 | (1) | 2 | (1) |

TABLE 12
Prespecified Overall Survival Sensitivity Analysis to Adjust
for the Effect of Subsequent Cellular Immunotherapy in the
Standard-Care Arm Using the Rank Preserving Structural Failure
Time Model. Kaplan-Meier estimate of overall survival with
sensitivity analysis using the Rank Preserving Structural Failure
Time method, which was performed to address the confounding
effect from treatment switching in the standard-care arm. The
treatment switching rate was defined as the proportion of patients
randomized to the standard-care arm who received commercially
available or investigational cellular immunotherapy after non-
response to or relapse after standard care. One-sided P value
from the log-rank test is reported.

| | Axicabtagene | Standard |
|---|---|---|
| | Ciloleucel | of Care |
| | (N = 180) | (N = 179) |
| Number of subjects | 180 | 179 |
| Number of subjects drop in from | — | 102 (57) |
| standard of care to CAR T-cell | | |
| therapy, n (%) | | |
| Stratified log-rank p-value adjusting | 0.0006 | — |
| for drop in | | |
| Hazard ratio (95% CI), stratified | 0.608 (0.449, | — |
| adjusting for drop in | 0.824) | |
| Unstratified log-rank p-value adjusting | 0.0018 | — |
| for drop in | | |
| Hazard ratio (95% CI), unstratified | 0.642 (0.475, | — |
| adjusting for drop in | 0.867) | |

Axi-cel, axicabtagene ciloleucel; HR, hazard ratio; NE, not estimable; NR, not reached; OS, overall survival.

Investigator-assessed PFS confirmed the benefit of axi-cel over standard care, with a median PFS of 14.7 months (95% CI, 5.4-43.5) with axi-cel and 3.7 months (95% CI, 2.9-5.3) with standard care (HR, 0.529 [95% CI, 0.383-0.669]; descriptive 1-sided P<0.0001) (Table 13). Estimated PFS rates at 48 months were 41.8% (95% CI, 34.1-49.2) with axi-cel and 24.4% (95% CI, 17.2-32.2) with standard care. Median investigator-assessed EFS, distinct from the primary endpoint of EFS per central review, was 10.8 months (95% CI, 5.0-25.5) with axi-cel and 2.3 months (95% CI, 1.7-3.1) with standard care, with estimated 48-month EFS rates of 38.9% and 17.3%, respectively (HR, 0.422 [95% CI, 0.327-0.545]; descriptive 1-sided P<0.0001) (Table 14).

Tables 13 and 14 below show progression-free survival and event-free survival as assessed by the investigator. Table 13 shows Kaplan-Meier estimate of progression-free survival, defined as the time from randomization to the date of disease progression or death from any cause, as assessed by the investigator. Table 14 shows Kaplan-Meier estimate of EFS (defined as the time from randomization to the earliest date of disease progression according to the Lugano classification, the commencement of new therapy for lymphoma, or death from any cause), as assessed by the investigator. For both Tables 13 and 14, patients who did not meet the criteria for an event had their data censored. Stratified Cox regression models were used to provide the estimated hazard ratios and 2-sided 95% confidence intervals for axi-cel relative to standard care hazard ratio. The Breslow method was used to handle the ties for the Cox regression models. One-sided P value from the log-rank test is reported. Axi-cel, axicabtagene ciloleucel; EFS, event-free survival; HR, hazard ratio; PFS, progression-free survival.

TABLE 13

| | Standard of | |
|---|---|---|
| Axi-Cel (n = 180) | Care (n = 179) | |
| NR (28.6-NE) | | Median OC (95%, CI), mo |
| 0.506 (0.383-0.669) | | Stratified HR (95% CI) |
| <0.0001 | | Stratified P = Value |
| 52 | 28 | 1-year OS Estimate (%) |
| 56 | 27 | 2-year OS Estimate (%) |
| 44 | 26 | 3-year OS Estimate (%) |
| 42 | 24 | 4-year OS Estimate (%) |

TABLE 14

| | Standard of | |
|---|---|---|
| Axi-Cel (n = 180) | Care (n = 179) | |
| NR (28.6-NE) | | Median OC (95%, CI), mo |
| 0.422 (0.327-0.545) | | Stratified HR (95% CI) |
| <0.0001 | | Stratified P = Value |
| 49 | 20 | 1-year OS Estimate (%) |
| 44 | 19 | 2-year OS Estimate (%) |
| 41 | 19 | 3-year OS Estimate (%) |
| 39 | 17 | 4-year OS Estimate (%) |

### Safety

The safety analysis set included 170 patients treated with axi-cel and 168 with standard care. All patients reported ≥1 treatment-emergent AE (TEAL); cumulative any-grade TLALs and grade≥3 TLALs, and serious TLALs are shown in Table 15 and Table 16, respectively. In the safety analysis set, 74 patients in the axi-cel arm and 91 patients in the standard-care arm died since the start of the study.

TABLE 15
Most Common Adverse Events, Cytokine Release Syndrome, and Neurologic Events.

| | Axi-Cel | | | | Standard Care | | | |
|---|---|---|---|---|---|---|---|---|
| | N = 170 | | | | N = 168 | | | |
| Event | Any Grade | | Grade ≥3 | | Any Grade | | Grade ≥3 | |
| Any adverse event, n (%) | 170 | (100) | 155 | (91) | 168 | (100) | 140 | (83) |
| Pyrexia | 158 | (93) | 15 | (9) | 43 | (26) | 1 | (1) |
| Neutropenia | 121 | (71) | 118 | (69) | 70 | (42) | 69 | (41) |
| Hypotension | 75 | (44) | 19 | (11) | 25 | (15) | 5 | (3) |
| Anemia | 71 | (42) | 51 | (30) | 91 | (54) | 65 | (39) |
| Fatigue | 71 | (42) | 11 | (6) | 87 | (52) | 4 | (2) |
| Diarrhea | 71 | (42) | 4 | (2) | 66 | (39) | 7 | (4) |
| Headache | 70 | (41) | 5 | (3) | 43 | (26) | 3 | (2) |
| Nausea | 69 | (41) | 3 | (2) | 116 | (69) | 9 | (5) |
| Sinus tachycardia | 58 | (34) | 3 | (2) | 17 | (10) | 1 | (1) |
| Leukopenia | 55 | (32) | 50 | (29) | 43 | (26) | 37 | (22) |
| Thrombocytopenia | 50 | (29) | 25 | (15) | 101 | (60) | 95 | (57) |
| Chills | 47 | (28) | 1 | (1) | 14 | (8) | 0 | (0) |
| Hypophosphatemia | 45 | (26) | 31 | (18) | 29 | (17) | 21 | (13) |
| Hypokalemia | 44 | (26) | 10 | (6) | 49 | (29) | 11 | (7) |
| Tremor | 44 | (26) | 2 | (1) | 1 | (1) | 0 | (0) |
| Decreased appetite | 42 | (25) | 7 | (4) | 42 | (25) | 6 | (4) |
| Cough | 42 | (25) | 1 | (1) | 18 | (11) | 0 | (0) |
| Confusional state | 40 | (24) | 9 | (5) | 4 | (2) | 0 | (0) |
| Hypoxia | 37 | (22) | 16 | (9) | 13 | (8) | 7 | (4) |
| Aphasia | 36 | (21) | 12 | (7) | 0 | (0) | 0 | (0) |
| Dizziness | 36 | (21) | 2 | (1) | 21 | (13) | 1 | (1) |
| Constipation | 34 | (20) | 0 | (0) | 58 | (35) | 0 | (0) |
| Vomiting | 33 | (19) | 0 | (0) | 55 | (33) | 1 | (1) |
| Hypomagnesemia | 20 | (12) | 1 | (1) | 34 | (20) | 4 | (2) |
| Febrile neutropenia | 6 | (4) | 6 | (4) | 46 | (27) | 46 | (27) |
| Cytokine release syndrome, n (%) | 157 | (92) | 11 | (6) | — | | — | |
| Pyrexia, n/total no. (%) | 155/157 | (99) | 14/157 | (9) | — | | — | |
| Hypotension, n/total no. (%) | 68/157 | (43) | 18/157 | (11) | — | | — | |
| Sinus tachycardia, n/total no. (%) | 49/157 | (31) | 3/157 | (2) | — | | — | |
| Chills, n/total no. (%) | 38/157 | (24) | 0/157 | (0) | — | | — | |
| Headache, n/total no. (%) | 32/157 | (20) | 2/157 | (1) | — | | — | |
| Hypoxia, n/total no. (%) | 31/157 | (20) | 13/157 | (8) | — | | — | |
| Neurologic event, n (%) | 103 | (61) | 36 | (21) | 33 | (20)^{¶} | 1 | (1) |
| Tremor | 44 | (26) | 2 | (1) | 1 | (1) | 0 | (0) |
| Confusional state | 40 | (24) | 9 | (5) | 4 | (2) | 0 | (0) |
| Aphasia | 36 | (21) | 12 | (7) | 0 | (0) | 0 | (0) |
| Encephalopathy | 29 | (17) | 20 | (12) | 2 | (1) | 0 | (0) |
| Paresthesia | 8 | (5) | 1 | (1) | 14 | (8) | 0 | (0) |
| Delirium | 3 | (2) | 3 | (2) | 5 | (3) | 1 | (1) |

TABLE 16
Serious Adverse Events Occurring in at Least
Three Patients in Either Treatment Arm.

| | Axi-Cel | | | | Standard Care | |
|---|---|---|---|---|---|---|
| | N = 170 | | | | N = 168 | |
| Event | Any Grade | | Grade ≥3 | | Any Grade | Grade ≥3 |
| Any, n (%) | 95 | (56) | 78 | (46) | 78 (46) | 68 (40) |
| Pyrexia | 27 | (16) | 1 | (1) | 8 (5) | 0 (0) |
| Encephalopathy | 17 | (10) | 15 | (9) | 1 (1) | 0 (0) |
| Hypotension | 15 | (9) | 7 | (4) | 3 (2) | 3 (2) |
| Pneumonia | 11 | (6) | 9 | (5) | 4 (2) | 3 (2) |
| Aphasia | 9 | (5) | 8 | (5) | 0 (0) | 0 (0) |
| B-cell lymphoma | 7 | (4) | 7 | (4) | 5 (3) | 5 (3) |
| Febrile neutropenia | 6 | (4) | 6 | (4) | 22 (13) | 22 (13) |
| Confusional state | 6 | (4) | 4 | (2) | 0 (0) | 0 (0) |
| COVID-19 | 6 | (4) | 3 | (2) | 1 (1) | 0 (0) |
| Neutropenia* | 6 | (4) | 5 | (3) | 4 (2) | 4 (2) |
| Atrial fibrillation | 5 | (3) | 4 | (2) | 2 (1) | 0 (0) |
| Somnolence | 5 | (3) | 3 | (2) | 0 (0) | 0 (0) |
| Tremor | 5 | (3) | 1 | (1) | 0 (0) | 0 (0) |
| Sepsis | 4 | (2) | 4 | (2) | 4 (2) | 4 (2) |
| Headache | 4 | (2) | 3 | (2) | 1 (1) | 1 (1) |
| Dyspnea | 3 | (2) | 3 | (2) | 1 (1) | 1 (1) |
| Acute kidney injury | 3 | (2) | 2 | (1) | 8 (5) | 4 (2) |
| Fatigue | 3 | (2) | 2 | (1) | 0 (0) | 0 (0) |
| Muscular weakness | 3 | (2) | 2 | (1) | 0 (0) | 0 (0) |
| Abdominal pain | 3 | (2) | 2 | (1) | 2 (1) | 1 (1) |
| Hypoxia | 3 | (2) | 1 | (1) | 2 (1) | 2 (1) |
| Anemia | 2 | (1) | 2 | (1) | 3 (2) | 3 (2) |
| Decreased appetite | 1 | (1) | 1 | (1) | 3 (2) | 3 (2) |
| Syncope | 1 | (1) | 1 | (1) | 3 (2) | 3 (2) |
| Thrombocytopenia^{†} | 0 | (0) | 0 | (0) | 6 (4) | 6 (4) |
| Dehydration | 0 | (0) | 0 | (0) | 3 (2) | 3 (2) |

Disease progression was the most common cause of death in both the axi-cel (n=51) and standard-care arms (n=71) (Table 17). Mortality related to the definitive therapy (axi-cel or HDT-ASCT) was 1/170 (1%) in the axi cel arm and 2/64 (3%) in the standard-care arm (Table 17). No changes in cumulative treatment-related serious AEs or fatal AEs occurred since the primary EFS analysis.

TABLE 17
Cumulative Deaths Among Treated Patients in the Axi-
Cel and Standard-Care Arms (Safety Analysis Set).

| | Axi-Cel | Standard Care |
|---|---|---|
| | N = 170 | N = 168 |
| Patients who died, n | 74 | 91 |

Reasons for death

| | | |
|---|---|---|
| Progressive disease, n | 51 | 71 |
| Fatal adverse event, n | 8 | 2 |
| COVID-19 | 2 | 0 |
| Sepsis | 2* | 0 |
| Acute respiratory distress syndrome | 0 | 1 |
| Cardiac arrest | 0 | 1 |
| Hepatitis B reactivation | 1 | 0 |
| Myocardial infarction | 1 | 0 |
| Pneumonia | 1 | 0 |
| Progressive multifocal leukoencephalopathy | 1 | 0 |
| New or secondary malignancy, n | 2^{†} | 0 |
| Other reason for death^{‡}, n | 13 | 18 |
| COVID-19 | 4 | 4 |
| Other infection/inflammation | 3 | 7^{§} |
| Neurologic organ failure | 2 | 0 |
| Respiratory organ failure | 1 | 1 |
| Cardiac organ failure | 1 | 0 |
| Cardiopulmonary/neurologic organ failure | 0 | 1 |
| Progressive disease | 1^{∥} | 0 |
| Unknown | 1 | 5 |
| Definitive therapy-related mortality, n/N (%) | 1/170 (1)^{¶} | 2/64 (3)^{#} |

New or secondary malignancies were reported in 11 patients (8 axi-cel, all investigator-assessed as unrelated to axi-cel; 3 standard care, including 1 patient with 2 new malignancies) since the start of study (Table 18). No cases of replication-competent retrovirus infection were reported.

TABLE 18
Listing of new of secondary malignancies

| | | | | Toxicity | Serious |
|---|---|---|---|---|---|
| Arm | Patient | Preferred Term | Causal Relationship* | Grade | AE |
| Axi-Cel | 1 | Spindle cell | None | 2 | Yes |
| | | sarcoma | | | |
| | 2 | Hepatocellular | None | 3 | Yes |
| | | carcinoma | | | |
| | 3 | Acute myeloid | Prior lymphoma-directed | 5 | No^{‡} |
| | | leukemia | chemotherapy, not | | |
| | | (transformed from | lymphodepleting | | |
| | | myelodysplastic | chemotherapy nor axi- | | |
| | | syndrome)^{†} | cel^{†} | | |
| | 4 | Lung | None | 5 | Yes |
| | | adenocarcinoma | | | |
| | 5 | Adenocarcinoma | None | 3 | Yes |
| | | of colon | | | |
| | 6 | Myelodysplastic | Lymphodepleting | 3 | Yes |
| | | syndrome^{§} | chemotherapy and prior | | |
| | | | lymphoma-directed | | |
| | | | chemotherapyⁱ | | |
| | 7 | Anal squamous | Lymphodepleting | 3 | Yes |
| | | cell carcinoma | chemotherapy | | |
| | 8 | Plasma cell | None | 1 | Yes |
| | | myeloma | | | |
| Standard | 9 | Ductal | None | 3 | Yes |
| Care | | adenocarcinoma of | | | |
| | | pancreas | | | |
| | 10 | Metastatic | None | 3 | Yes |
| | | malignant | | | |
| | | melanoma | | | |
| | 11 | Bladder | SOC chemotherapy, | 3 | Yes |
| | | transitional cell | possibly prior | | |
| | | carcinoma | chemotherapy | | |
| | | Adenocarcinoma | SOC chemotherapy, prior | 3 | Yes |
| | | of colon^{∥} | prostate cancer therapy | | |

Any-grade and grade≥3 infections were reported in 76 (45%) and 28 (16%) patients in the axi-cel arm, and in 53 (32%) and 20 (12%) in the standard-care arm, respectively (Table 19). Of 162 axi-cel-treated patients evaluated for B-cell levels, 47% had B-cell aplasia (undetectable B cells) up to 3 months post-infusion (Table 20; Table 21).

TABLE 19
Treatment-emergent Infections by Preferred Term and Grade.

| | Axi-Cel | Standard Care | |
|---|---|---|---|
| n (%) | N = 170 | N = 168 | |
| Any | 76 (45) | 53 | (32) |
| Grade ≥3 | 28 (16) | 20 | (12) |
| Grade 5 | 7 (4) | 0 | (0) |
| Bacterial infection | | | |
| Any | 18 (11) | 18 | (11) |
| Grade ≥3 | 8 (5) | 7 | (4) |
| Grade 5 | 0 (0) | 0 | (0) |
| Opportunistic infection | | | |
| Any | 8 (5) | 2 | (1) |
| Grade ≥3 | 2 (1) | 2 | (1) |
| Grade 5 | 1 (1) | 0 | (0) |
| Viral infection (including COVID-19) | | | |
| Any | 31 (18) | 10 | (6) |
| Grade ≥3 | 9 (5) | 2 | (1) |
| Grade 5 | 4 (2) | 0 | (0) |
| COVID-19 infection^{†} | | | |
| Any | 8 (5) | 2 | (1) |
| Grade ≥3 | 5 (3) | 0 | (0) |
| Grade 5^{‡} | 2 (1) | 0 | (0) |
| Unspecified infection | | | |
| Any | 49 (29) | 39 | (23) |
| Grade ≥3 | 20 (12) | 15 | (9) |
| Grade 5 | 3 (2) | 0 | (0) |

TABLE 20
Summary of B-cell Aplasia by Baseline Status.

| | Axi-Cel |
|---|---|
| | N = 162 |
| Overall, n | 162 |
| No B cells up to 3 months postinfusion, n (%) | 76 (47) |
| No B cells up to 6 months postinfusion, n (%) | 75 (46) |
| No B cells up to 9 months postinfusion, n (%) | 61 (38) |
| No B cells up to 12 months postinfusion, n (%) | 57 (35) |
| No B cells up to 18 months postinfusion, n (%) | 55 (34) |
| No B cells up to 24 months postinfusion, n (%) | 52 (32) |
| No B cells at baseline, n | 60 (37) |
| No B cells up to 3 months postinfusion, n (%) | 37 (23) |
| No B cells up to 6 months postinfusion, n (%) | 36 (22) |
| No B cells up to 9 months postinfusion, n (%) | 31 (19) |
| No B cells up to 12 months postinfusion, n (%) | 30 (19) |
| No B cells up to 18 months postinfusion, n (%) | 28 (17) |
| No B cells up to 24 months postinfusion, n (%) | 27 (17) |
| With B cells at baseline, n | 83 (51) |
| No B cells up to 3 months postinfusion, n (%) | 31 (19) |
| No B cells up to 6 months postinfusion, n (%) | 31 (19) |
| No B cells up to 9 months postinfusion, n (%) | 24 (15) |
| No B cells up to 12 months postinfusion, n (%) | 22 (14) |
| No B cells up to 18 months postinfusion, n (%) | 22 (14) |
| No B cells up to 24 months postinfusion, n (%) | 20 (12) |

TABLE 21
CAR T-cell and B-cell Levels Over Time. Log median (±95%
CI) percentage of B cells out of total leukocytes and the number
of anti CD19 CAR T cells in blood over time, from baseline
to 24 months after axi-cel infusion, for evaluable patients.
B-Cell Levels over time

| | | | | | n of |
|---|---|---|---|---|---|
| Time | | | Upper | Lower | evaluable |
| point | Months | Median | limit | limit | patients |
| Baseline | −1 | 0.030162 | 32.17964 | 0.017 | 143 |
| MONTH 3 | 3 | 0.017 | 24.50566 | 0.017 | 138 |
| MONTH 6 | 6 | 0.017 | 22.99639 | 0.017 | 18 |
| MONTH 9 | 9 | 0.071541 | 49.59918 | 0.017 | 78 |
| MONTH 12 | 12 | 0.07734 | 52.88231 | 0.017 | 75 |
| MONTH 18 | 18 | 7.65575 | 54.77251 | 0.017 | 64 |
| MONTH 24 | 24 | 11.29705 | 48.99476 | 0.017 | 53 |

CAR T-cell numbers over time

| | | Upper | Lower | n of evaluable |
|---|---|---|---|---|
| Months | Median | limit | limit | patients |
| −1 | 0 | 0 | 0 | 165 |
| 0.033333 | 0.004061 | 22.67582 | 0.000162 | 146 |
| 0.1 | 0.01425 | 8.2071 | 0.001 | 45 |
| 0.233333 | 21.36624 | 1173.247 | 0.001 | 156 |
| 0.466667 | 6.280313 | 110.6028 | 0.10535 | 25 |
| 0.933333 | 1.596602 | 128.5843 | 0.001 | 129 |
| 3 | 0.351433 | 28.44498 | 0.000315 | 143 |
| 6 | 0.166622 | 14.90976 | 0.001 | 19 |
| 9 | 0.139082 | 2.9529 | 0.001 | 77 |
| 12 | 0.0845925 | 2.36379 | 0.001 | 74 |
| 18 | 0.020261 | 2.572479 | 0.001 | 68 |
| 24 | 0.001 | 1.337767 | 0.001 | 57 |

Axi-cel, axicabtagene ciloleucel; BLQ, below the limit of quantitation; CAR, chimeric antigen receptor.

B-cell recovery (detectable B-cell levels compared with prior timepoints) was observed over time with wide interpatient variability. Median B-cell levels were at or below the lower limit of quantitation (0.017%) until Month 6 after infusion and started to increase at Month 9, which coincided with the disappearance of or very low levels of CAR T-cells in blood (median around or below 0.1 cells/μL; Table 21, see above).

Hypogammaglobulinemia was reported in 11% and 1% of patients in the axi cel and standard-care arms, respectively; all cases were grade 1-2 (Table 22). Twenty-eight (16%) patients in the axi-cel arm received intravenous immunoglobulin therapy per investigator discretion (Table 22).

TABLE 22
Treatment-emergent Hypogammaglobulinemia
by Worst Grade and IVIG Use.

| | Axi-Cel | | SOC | Overall | |
|---|---|---|---|---|---|
| n (%) | N = 170 | | N = 168 | N = 338 | |
| All treatment-emergent | 19 | (11) | 1 (1) | 20 | (6) |
| hypogammaglobulinemia | | | | | |
| Grade 1 | 6 | (4) | 1 (1) | 7 | (2) |
| Grade 2 | 13 | (8) | 0 (0) | 13 | (4) |
| Grade ≥3 | 0 | (0) | 0 (0) | 0 | (0) |

| | | | | |
|---|---|---|---|---|
| Received IVIG (intravenous | 28 | (16) | — | — |

immunoglobin) therapy

Grade≥3 prolonged cytopenia, thrombocytopenia, and neutropenia were reported by 8 (5%), 0 (0%), and 6 (4%) of patients, respectively, in the axi-cel arm (N=170) and 1 (2%), 1 (2%), and 0 (0%) patients, respectively, in the standard-care arm (n=62) on or after 6 months from initiation of definitive therapy (Table 23).

TABLE 23
Summary of Cytopenias Present on or After 6, 12, 18, and 24
Months From Initiation of Definitive Therapy on Protocol.

| | | | | Standard Care Patients | |
|---|---|---|---|---|---|
| | Axi-Cel | | | Who Proceeded to ASCT | |
| | N = 170 | | | n = 62 | |
| | Any | | | Any | |
| n (%) | Grade | | Grade ≥3 | Grade | Grade ≥3 |
| Prolonged cytopenia^{†} | | | | | |
| 6 months | 19 | (11) | 8 (5) | 4 (6) | 1 (2) |
| 12 months | 11 | (6) | 4 (2) | 1 (2) | 0 (0) |
| 18 months | 5 | (3) | 4 (2) | 0 (0) | 0 (0) |
| 24 months | 3 | (2) | 2 (1) | 0 (0) | 0 (0) |
| Prolonged | | | | | |
| thrombocytopenia^{‡} | | | | | |
| 6 months | 6 | (4) | 0 (0) | 3 (5) | 1 (2) |
| 12 months | 3 | (2) | 0 (0) | 1 (2) | 0 (0) |
| 18 months | 0 | (0) | 0 (0) | 0 (0) | 0 (0) |
| 24 months | 0 | (0) | 0 (0) | 0 (0) | 0 (0) |
| Prolonged | | | | | |
| neutropenia^{§} | | | | | |
| 6 months | 9 | (5) | 6 (4) | 0 (0) | 0 (0) |
| 12 months | 3 | (2) | 2 (1) | 0 (0) | 0 (0) |
| 18 months | 2 | (1) | 2 (1) | 0 (0) | 0 (0) |
| 24 months | 2 | (1) | 2 (1) | 0 (0) | 0 (0) |
| Febrile neutropenia | | | | | |
| 6 months | 3 | (2) | 3 (2) | 0 (0) | 0 (0) |
| 12 months | 2 | (1) | 2 (1) | 0 (0) | 0 (0) |
| 18 months | 2 | (1) | 2 (1) | 0 (0) | 0 (0) |
| 24 months | 2 | (1) | 2 (1) | 0 (0) | 0 (0) |
| Prolonged anemia^{∥} | | | | | |
| 6 months | 8 | (5) | 2 (1) | 1 (2) | 0 (0) |
| 12 months | 6 | (4) | 2 (1) | 0 (0) | 0 (0) |
| 18 months | 3 | (2) | 2 (1) | 0 (0) | 0 (0) |
| 24 months | 1 | (1) | 0 (0) | 0 (0) | 0 (0) |

No new CRS or neurologic events were reported in either treatment arm since the primary EFS analysis (Table 15, see above).

### Exploratory Translational Analyses

Peak CAR T-cell levels and area under the curve within the first 28 days after infusion were not significantly associated with OS (Tables 24 and 25). In axi-cel-treated patients, OS benefit was independent of axi-cel product characteristics with two notable exceptions (Table 26).

Tables 24 and 25 show the association between overall survival with CAR T-cell expansion. Kaplan-Meier estimate and estimated hazard ratio of overall survival in axi-cel patients by postinfusion (Table 24) CAR T-cell peak levels (defined as the maximum number of CAR T cells in blood after axi-cel infusion) and (Table 25) AUC0-28. CAR T-cell expansion was divided into subgroups based on median value. Stratified Cox regression models were used to provide the estimated hazard ratios and 2-sided 95% confidence intervals for axi-cel peak >median relative to peak ≤median. The Breslow method was used to handle the ties for the Cox regression models. One sided P value from log-rank test is presented. AUC0-28, area under the curve from day 0 to day 28; axi-cel, axicabtagene ciloleucel; CAR, chimeric antigen receptor; HR, hazard ratio; OS, overall survival.

TABLE 24
Overall Survival by Median of Peak of Anti-CD19
CAR T Cell Levels in Blood (Safety Analysis Set)

| | Axicabtagene Ciloleucel | |
|---|---|---|
| | (N = 170) | |
| | Peak (cells/μL) > | Peak (cells/μL) ≤ |
| | Median | Median |
| | (N = 81) | (N = 82) |
| Number of subjects | 81 | 82 |
| Events, n (%) | 36 (44) | 35 (43) |
| Censored, n (%) | 45 (56) | 47 (57) |
| Stratified (derived) log- | 0.3409 | NA |
| rank p-value | | |
| Hazard ratio (95% CI), | 0.91 (0.57, | NA |
| stratified (derived) | 1.45) | |
| Unstratified log-rank p-value | 0.4607 | NA |
| Hazard ratio (95% CI), | 0.98 (0.61, | NA |
| unstratified | 1.56) | |
| KM median (95% CI) OS | NE (28.29, | NE (25.53, |
| time (months) | NE) | NE) |
| Min, Max OS time (months) | 3, 57 | 3, 60 |

TABLE 25
Overall Survival by Median of AUC of Anti-CD19 CAR
T Cell Levels in Blood (Safety Analysis Set)

Axicabtagene Ciloleucel
(N = 170)

| | AUC₀₋₂₈ (cells/ | AUC₀₋₂₈ (cells/ |
|---|---|---|
| | μL*days) > | μL*days) ≤ |
| | Median | Median |
| | (N = 81) | (N = 82) |
| Number of subjects | 81 | 82 |
| Events, n (%) | 36 (44) | 35 (43) |
| Censored, n (%) | 45 (56) | 47 (57) |
| Stratified (derived) log- | 0.3750 | NA |
| rank p-value | | |
| Hazard ratio (95% CI), | 0.93 (0.58, | NA |
| stratified (derived) | 1.49) | |
| Unstratified log-rank p-value | 0.4718 | NA |
| Hazard ratio (95% CI), | 1.02 (0.64, | NA |
| unstratified | 1.62) | |
| KM median (95% CI) OS | NE (25.26, | NE (25.56, |
| time (months) | NE) | NE) |
| Min, Max OS time (months) | 3, 59 | 3, 60 |

TABLE 26
Association Between Axi-Cel Product Characteristics
and Overall Survival by Time to Event.

| | Axi-Cel (N = 170)* | | |
|---|---|---|---|
| | No. of | Hazard | |
| | Evaluable | Ratio | P |
| Characteristic | Patients | (95% CI) | Value |
| Viability, % | 170 | 0.78 (0.48- | 0.3200 |
| | | 1.27) | |
| Transduction rate, % | 170 | 1.26 (0.79- | 0.3324 |
| | | 1.99) | |
| CD3+ cells, % | 166 | 1.10 (0.70- | 0.6740 |
| | | 1.75) | |
| CD4+ cells, % of viable CD3+ cells | 166 | 0.92 (0.58- | 0.7386 |
| | | 1.47) | |
| CD8+ cells, % of viable CD3+ cells | 166 | 1.08 (0.68- | 0.7386 |
| | | 1.72) | |
| CD4/CD8 ratio | 166 | 0.92 (0.58- | 0.7386 |
| | | 1.47) | |
| Tₙₐᵢᵥₑ cells, % of viable CD3+ cells | 166 | 0.57 (0.36- | 0.0204^{†} |
| | | 0.92) | |
| T_{EFF}, % of viable CD3+ cells | 166 | 1.23 (0.77- | 0.3886 |
| | | 1.95) | |
| T_{EM}, % of viable CD3+ cells | 166 | 1.76 (1.10- | 0.0176^{†} |
| | | 2.82) | |
| T_{CM}, % of viable CD3+ cells | 166 | 1.23 (0.77- | 0.3889 |
| | | 1.95) | |
| (Tₙₐᵢᵥₑ + T_{CM})/(T_{EM} + T_{EFF}) ratio | 166 | 0.75 (0.47- | 0.2228 |
| | | 1.19) | |
| CCR7+ cells (Tₙₐᵢᵥₑ + T_{CM}), % | 166 | 0.75 (0.47- | 0.2228 |
| | | 1.19) | |
| CCR7− cells (T_{EM} + T_{EFF}), % | 166 | 1.34 (0.84- | 0.2228 |
| | | 2.13) | |
| Total number of CD3+ T cells | 166 | 1.13 (0.71- | 0.6155 |
| infused, ×10⁶ | | 1.79) | |
| Total number of CD4+ T cells | 166 | 0.98 (0.62- | 0.9212 |
| infused, ×10⁶ | | 1.55) | |
| Total number of CD8+ T cells | 166 | 0.97 (0.61- | 0.9005 |
| infused, ×10⁶ | | 1.54) | |
| Total number of Tₙₐᵢᵥₑ cells | 166 | 0.73 (0.46- | 0.1770 |
| infused, ×10⁶ | | 1.16) | |
| Total number of T_{EFF} cells | 166 | 1.26 (0.79- | 0.3367 |
| infused, ×10⁶ | | 2.00) | |
| Total number of T_{EM} cells | 166 | 1.24 (0.78- | 0.3716 |
| infused, ×10⁶ | | 1.96) | |
| Total number of T_{CM} cells | 166 | 1.21 (0.76- | 0.4162 |
| infused, ×10⁶ | | 1.93) | |
| Total number of CCR7+ T cells | 166 | 0.88 (0.55- | 0.5858 |
| infused, ×10⁶ | | 1.40) | |
| Total number of CCR7− T cells | 166 | 1.35 (0.85- | 0.2100 |
| infused, ×10⁶ | | 2.14) | |
| CD3− cells, % | 166 | 0.91 (0.57- | 0.6740 |
| | | 1.44) | |
| Total number of CD3− cells | 166 | 0.97 (0.61- | 0.9114 |
| infused, ×10⁶ | | 1.55) | |
| Total number of T cells | 170 | 1.22 (0.77- | 0.4048 |
| infused, ×10⁶ | | 1.92) | |
| IFN-γ level, pg/mL | 170 | 0.96 (0.60- | 0.8548 |
| | | 1.52) | |

In the analysis of OS versus product features, improved OS was associated with greater proportion of more juvenile or stem memory T cell phenotype (CCR7+CD45RA+ T cells) in the axi-cel product (stratified descriptive P=0.0085) (not shown). Conversely, worse OS was associated with a greater proportion of differentiated T cells, specifically effector memory (CCR7−CD45RA−), in the axi-cel product (stratified descriptive PH0.0091) (not shown). Table 27 below shows various product characteristics of the ax-cel product. More specifically, an increased overall survival rate was observed in patients having an increased percentage of juvenile or stem memory T cell phenotype (i.e. naïve T-cells) versus the median value for juvenile or stem memory T cells in an axi-cel product, while an overall decreased survival rate was observed in patients having an increased percentage of differentiated T cells versus the median value for differentiated T cells in an axi-cel product.

TABLE 27
Axi-Cel Product Characteristics

| | Axicabtagene Ciloleucel (N = 170) | | | | | | |
|---|---|---|---|---|---|---|---|
| | | | | Hazard Ratio | Stratified | | |
| | Number of | Events, | Censored, | (95% CI), | (derived) | Hazard Ratio | Unstratified |
| Product | Evaluable | n | n | Stratified | Cox PH | (95% CI) | Cox PH |
| Characteristic | Subjects | (%) | (%) | (derived) | P-value | Unstratified | P-value |
| CD4/CD8 Ratio | | | | | | | |
| >Median | 83 | 37 | 46 | 0.92 | 0.7386 | 0.99 | 0.9513 |
| | | (44.6) | (55.4) | (0.58, 1.47) | | (0.62, 1.56) | |
| <=Median | 83 | 36 | 47 | | | | |
| | | (43.4) | (56.6) | | | | |
| Tnaive (% of | | | | | | | |
| viable CD3+ cells) | | | | | | | |
| >Median | 83 | 29 | 54 | 0.57 | 0.0204 | 0.57 | 0.0174 |
| | | (34.9) | (65.1) | (0.36, 0.92) | | (0.35, 0.90) | |
| <=Median | 83 | 44 | 39 | | | | |
| | | (53.0) | (47.0) | | | | |
| Teff (% of viable | | | | | | | |
| CD3+ cells) | | | | | | | |
| >Median | 83 | 38 | 45 | 1.23 | 0.3886 | 1.16 | 0.5269 |
| | | (45.8) | (54.2) | (0.77, 1.95) | | (0.73, 1.84) | |
| <=Median | 83 | 35 | 48 | | | | |
| | | (42.2) | (57.8) | | | | |
| Tem (% of viable | | | | | | | |
| CD3+ cells) | | | | | | | |
| >Median | 82 | 43 | 39 | 1.76 | 0.0176 | 1.72 | 0.0224 |
| | | (52.4) | (47.6) | (1.10, 2.82) | | (1.08, 2.75) | |
| <=Median | 84 | 30 | 54 | | | | |
| | | (35.7) | (64.3) | | | | |
| Tcm (% of viable | | | | | | | |
| CD3+ cells) | | | | | | | |
| >Median | 82 | 40 | 42 | 1.23 | 0.3889 | 1.29 | 0.2744 |
| | | (48.8) | (51.2) | (0.77, 1.95) | | (0.82, 2.05) | |
| <=Median | 84 | 33 | 51 | | | | |
| | | (39.3) | (60.7) | | | | |

### Further Results

### Preplanned Interim OS Analysis Results

As previously reported, a preplanned interim analysis of OS occurred at the time of the primary EFS analysis in ZUMA-7. In the intention-to-treat analysis set, median OS was not reached in the axi-cel arm and was 35.1 months in the standard-care arm (hazard ratio [HR], 0.73; 95% CI, 0.53-1.01), favoring axi-cel. Following a United States Food and Drug Administration request for additional survival follow-up for discontinued patients, the interim OS analysis was updated to include information from public records prior to the Mar. 18, 2021, data cutoff date. In that analysis, the median OS was not reached in the axi-cel arm and was 25.7 months in the standard-care arm (HR, 0.71; 95% CI, 0.52-0.97).

### Prespecified Sensitivity Analyses of OS

In a prespecified sensitivity analyses designed to assess the confounding effect of treatment switching on OS in the standard-care arm, axi-cel showed significantly longer median OS than standard care using the Rank Preserving Structural Failure Time model (not reached [95% CI, 28.6 months—not estimable] versus 15.5 months [95% CI, 9.7—not estimable]; HR, 0.608 [95% CI, 0.449-0.824)]; stratified log-rank 1-sided descriptive P=0.0006) (Table 12). Similar results were obtained using Inverse Probability of Censoring Weights methodology (HR, 0.633; 95% CI, 0.438-1.118).

### Additional Response Outcomes

In the present analysis, the objective response rate per investigator was 83% (61% complete response rate) in the axi-cel arm and 45% (34% complete response rate) in the standard-care arm. The median duration of response was 41.7 months (95% CI, 13.6—not estimable) and 7.8 months (95% CI, 5.0—not estimable), in the axi-cel and standard-care arms, respectively. At time of data cutoff, 71/180 (39%) and 29/179 patients (16%), respectively, had an ongoing response.

### Additional Safety Outcomes

There were two patients who had ongoing neurologic events at the time of the previous analysis: one patient randomized to the axi-cel arm had grade 2 paresthesia and grade 1 memory impairment, with both symptoms persistent until the patient died; one patient randomized to the standard-care arm had grade 1 paresthesia that remained an ongoing symptom in long-term follow-up.

### Discussion

In this trial comparing two second-line treatment strategies for patients with R/R LBCL, the risk of death with axi-cel was significantly lower by 27.4% versus standard care, with an 8.6% absolute improvement in survival at 4 years.

Prior to the advent of CAR T-cell therapy, patients with second-line LBCL who were unable to proceed to definitive therapy with HDT-ASCT had poor outcomes, with a median OS of 4.4 months. Previous attempts to improve survival for patients in the second-line curative setting had been unsuccessful, implying a maximum benefit of chemotherapy-based approaches had been reached; the most recent trial to demonstrate a survival improvement was a 1995 Parma study, which was conducted prior to the approval of rituximab. Thus, a new non-chemotherapy-based second-line approach was needed in high-risk patients with early R/R LBCL, prompting the design of the ZUMA 7 study.

Axi-cel was the first CAR T-cell therapy to receive regulatory approval for second-line therapy for LBCL in the United States, European Union, and many other countries, based on superior EFS and response with axi-cel over chemotherapy/HDT-ASCT in ZUMA-7. The results of the ZUMA-7 primary OS analysis reported herein now definitively demonstrate that patients who receive axi-cel have a clear survival advantage over patients who receive second-line platinum-based chemotherapy and, if responsive, HDT-ASCT. Importantly, the stability of OS and PFS Kaplan-Meier survival curves to 4 years suggests the curative potential of second-line axi-cel for a substantial proportion of patients.

The OS benefit of axi-cel over chemotherapy/HDT ASCT was consistent across key prespecified patient subgroups known to confer poor prognosis, including patients≥65 years old, those with disease refractory to first-line therapy, and those with HGBL. Axi-cel benefit in older patients is of particular interest, as patients who would otherwise be considered ineligible for definitive therapy with HDT-ASCT due to advanced age may still qualify for CAR T-cell therapy. Given the OS and EFS improvements reported with axi-cel in older patients, which were at least similar to those in the overall population, axi-cel may expand the patient population able to benefit from curative-intent therapy.

Furthermore, axi-cel was associated with a significant OS benefit despite more than half of patients in the standard-care arm receiving subsequent cellular immunotherapy off-protocol due to lack of response or disease progression. Notably, this rate was similar to other contemporary randomized anti-CD19 CAR T-cell therapy trials which included a protocol-specified crossover design. Historically, while standard-care patients with early relapse or with prior rituximab treatment showed estimated 3-year OS rates of 39% and 40%, respectively, those with both early relapse and prior rituximab, representing the patient population in ZUMA 7, had less than 40% 3-year survival. In contrast, standard-care patients in ZUMA-7 demonstrated an estimated 3-year OS rate of 48%. Given the increased survival in the standard-care arm compared with historical studies due to availability of third-line CAR T-cell therapy during the conduct of ZUMA-7, the true survival benefit of second-line axi-cel over standard care may be even greater, as supported by our treatment switching analyses. Based on the clear improvement in survival over platinum-based chemotherapy/HDT-ASCT, axi-cel should be a second-line standard treatment, as opposed to attempting second line chemotherapy and administering cellular immunotherapy only upon demonstration of lack of adequate response.

The long-term safety profile of axi-cel was consistent with prior studies. Of note, per protocol, the AE reporting period ended with disease progression or start of new lymphoma therapy, both of which were disproportionally higher on the standard-care arm. With CD19-targeted CAR T-cell therapy, prolonged cytopenia and immune deficiency, including induction of B-cell aplasia and infection, are anticipated, representing an on target/off-tumor class effect. Notably, the incidence of prolonged grade≥3 cytopenia decreased over time, beginning at 6 months after axi-cel infusion. Given the incidence of grade≥3 infections, hypogammaglobulinemia, and B cell aplasia with axi-cel, clinical monitoring of patients treated with CAR T-cell therapy is important to mitigate long-term infection risk. B-cell recovery was observed over time in the majority of axi-cel-treated patients, implying that durable clinical benefit is not dependent upon long-term persistence of functional CAR T cells, as previously described for axi-cel in the third-line or later setting.

In addition to improved survival, axi-cel was associated with improved quality-of-life (QoL). Axi-cel-treated patients had significantly longer time without symptoms or toxicity, with a clinically important gain in quality-adjusted survival, clinically meaningful improvements in QoL, and faster recovery to baseline versus standard care.

Importantly, OS is an objective endpoint that does not suffer from observer bias, and the ZUMA-7 primary OS analysis definitively establishes that the treatment strategy of second line axi-cel for early R/R LBCL is superior to chemotherapy followed in responding patients by HDT-ASCT. With platinum chemotherapy as initial second-line treatment, only a small minority of patients receive definitive therapy with HDT-ASCT, mainly due to lack of chemosensitivity (which is unknown prior to treatment initiation). The improvement in OS with axi-cel underscores the importance of early referral for axi-cel prior to the initiation of second line chemotherapy.

ZUMA-7 demonstrated a significant improvement in survival with axi cel compared with second-line chemotherapy/HDT-ASCT in patients with R/R LBCL, with long-term outcomes consistent with curative therapy.

### Example 3

This example discloses classification modeling to identify patients at risk for neurotoxicity and CRS following axicabtagene ciloleucel (axi-cel) treatment in 2nd line R/R LBCL.

Axi-cel is an autologous anti-CD19 CAR T-cell therapy approved for relapsed/refractory (R/R) large B-cell lymphoma (LBCL) after ≥1 lines of systemic therapy. Superiority over standard of care in 2nd line (refractory or early relapse) was shown in the randomized Phase 3 Zuma-7 trial. Post-axi-cel cytokine release syndrome (CRS) and neurologic events (NE) were reported in 92% and 61% of patients; rates of high-grade (Grade 3+) events were 6% and 21%, respectively. The pathophysiology of CRS is well documented, while the mechanisms underlying NE remain elusive. There is interest in prospectively identifying patients at low risk for toxicity who may be treated in the outpatient setting.

Here, an objective was to create data-driven, multivariable classification models in 2^{nd} line R/R LBCL using pre-treatment biomarkers to identify patients with the following toxicity-related outcomes: Grade 3+(G3+) NE at any time post-axi-cel, and Low grade or no toxicity (Maximum of Grade 1 CRS and no NE within 7 days post-axi-cel). We also sought to validate top models in multiple R/R LBCL populations as identified in Table 28 below.

We incorporated ˜450 biomarker+timepoint combinations. Candidate biomarkers included: Demographics (˜5), clinical and disease characteristics (˜30), routine serum chemistry and hematology (˜30), cytokines and other pharmacodynamic markers (˜25), and product attributes (˜30). Timepoints for measuring these biomarkers included: Baseline (i.e., prior to lymphodepletion), Day 0 (prior to axi-cel treatment); also considered Fold Change from Baseline to Day 0, calculated as (Day 0)/(Baseline). Machine learning methods (e.g., conditional random forest, XGBoost) were used to select features. Using selected features, logistic classifiers were trained and performance was evaluated in the hold-out test set (70:30% split). Bootstrap resamples were used to estimate variability. A data-driven approach was implemented (i.e., a set of presumed clinically-relevant biomarkers was not selected a priori). Clinician-curated models were selected from top data-driven models.

TABLE 28
Sample size and outcome frequency

| | | Zuma 1 | Zuma | | Zuma 1 | |
|---|---|---|---|---|---|---|
| | Zuma 7 | Cohorts 1-2 | 1Cohort 4 | | Cohort 6 | |
| Number of | 170 | 101 | 41 | | 40 | |
| patients treated | | | | | | |
| with axi-cel | | | | | | |
| Grade 3+ NE at | 36 (21%) | 31 (31%) | 7 | (17%) | 7 | (17%) |
| anytime | | | | | | |
| Low Grade of | 67 (39%) | 26 (26%) | 10 | (24%) | 15 | (38%) |

No toxicity
within 7 days

Table 29 shows results for the top models for predicting Grade 3+ neurotoxicity identified using data-driven approach. Selection was based on high area under the receiver operating characteristic (AUROC), high negative predictive value (NPV), and consistent covariate directionality for train and test sets. In Table 29, the higher the level of the biomarker, the higher the likelihood of occurrence of Grade 3+ neurotoxicity.

TABLE 29
Top data-driven models for Grade 3+ neurotoxicity

| Model | Biomarkers and timepoints |
|---|---|
| 1 | Calcium Fold Change between Baseline and Day 0 + IFN-g Normalized by |
| | Transduction Rate (in product) + Amyloid A at Day 0 (Higher vs. Lower |
| | than median) + Stratum 3 (1 L relapse 6-12 months and 2 L Age-adj IPI 0-1) |
| 2 | Amyloid A at Day 0 (Higher vs. Lower than median) + Calcium at Day 0 + |
| | IFN-g Normalized by Transduction Rate (in product) + VCAM-1 at |
| | Baseline + Activated B-cell Subtype (Y/N) |
| 3 | IL-15 at Day 0 + Calcium Fold Change between Baseline and Day 0 + Bone |
| | Marrow Involvement (Y/N) + WBC at Day 0 |
| 4 | IL-7 Fold Change between Baseline and Day 0 + IL-15 Fold Change |
| | between Baseline and Day 0 + Bone Marrow Involvement (Y/N) + Calcium |
| | Fold Change between Baseline and Day 0 + Sodium at Baseline |
| 5 | ICAM-1 at Day 0 (Higher vs. Lower than median) + Calcium at Baseline + |
| | Calcium at Day 0 + IFN-g Normalized by Transduction Rate (in product) |
| 6 | Clinical CRP at Day 0 + Calcium Fold Change between Baseline and Day |
| | 0 + Activated B-cell Subtype (Y/N) + IFN-g Normalized by Transduction |
| | Rate (in product |

Table 30 shows results for the top data-driven models for low grade or no toxicity. Selection was based on high AUROC, high PPV, and consistent covariate directionality for train and test sets. In Table 30, the higher the level of the biomarker, the higher the likelihood of having low or no toxicity.

TABLE 30
Top data-driven models for low grade or no toxicity

| Model | Biomarkers and timepoints |
|---|---|
| 1 | Teff (% viable CD3+ cells in product) + IL-15 Fold Change between |
| | Baseline and Day 0 + Stratum 3 (1 L relapse 6-12 months and 2 L Age- |
| | Adjusted IPI 0-1) |
| 2 | MCP-4 Fold Change between Baseline and Day 0 (DM) + Sodium at |
| | Baseline (DM) + IL-15 at Day 0 + IL-6 at Baseline (DM) + IL-10 Fold |
| | Change between Baseline and Day 0 (DM) + CCR7+ (Tnaive + Tcm in |
| | product) (%) |
| 3 | Teff (% viable CD3+ cells in product) + IL-15 at Day 0 + IL-2R at Baseline |
| 4 | Total Number of Teff Cells Infused (in product, ×106) (DM) + Chloride at |
| | Baseline (DM) + Clinical CRP at Day 0 (DM) + IFN-g at Day 0 (DM) + |
| | Stratum 3 (1 L relapse 6-12 months and 2 L Age-Adjusted IPI 0-1) |
| 5 | IL-10 at Baseline (DM) + IL-15 at Day 0 + CCR7− (Tem + Teff in product) |
| | (%) + Creatinine at Day 0 (DM) |
| 6 | Chloride at Baseline (DM) + IL-15 at Day 0 + Amyloid A at Day 0 (DM) + |
| | Total Number of Tcm Cells Infused (in product, ×106) (DM) |

While it remains a challenge to predict which patients will experience high-grade NE toxicity, the present results show good performance in prospectively identifying a subset of patients who are not likely to experience high-grade NE toxicity (i.e., high NPV). The disclosed results include a best-performing classifier for low grade or no toxicity (defined by ≤Grade 1 CRS and no NE) achieved a PPV in training & test sets of 70% (7 of 10 correctly classified), suggesting a possible avenue to identify patients for outpatient administration. This modeling work has utility in supporting clinicians in prospectively managing patients who may be at risk for toxicity, as well as those patients for whom outpatient monitoring may be desirable.

### Example 4

Axicabtagene ciloleucel (axi-cel) is an autologous anti-CD19 CAR T-cell therapy approved for relapsed/refractory (R/R) follicular lymphoma (FL). Approval was supported by the phase 2, multicenter, single-arm ZUMA-5 study of axi-cel in patients with R/R indolent non-Hodgkin lymphoma (iNHL; N=104) including FL and marginal zone lymphoma [MZL]. In the primary analysis (17.5 months median follow-up), overall response rate (ORR) was 92% (74% complete response rate). Here we report long-term outcomes from ZUMA-5. Eligible patients with R/R iNHL after 2 lines of therapy underwent leukapheresis, followed by lymphodepleting chemotherapy and axi-cel infusion (2×10⁶ CAR T cells/kg). The primary endpoint was ORR, assessed in this analysis by investigators in all enrolled patients (intent-to-treat). After median follow-up of 41.7 months in FL (n=127) and 31.8 months in MZL (n=31), ORR was comparable to the primary analysis (94% in FL; 77% in MZL). Median progression-free survival was 40.2 months in FL and not yet reached in MZL. Medians of overall survival were not reached in either disease type. Grade≥3 adverse events of interest occurring since the prior analysis were largely in recently treated patients. Clinical and pharmacokinetic outcomes correlated negatively with recent exposure to bendamustine and high metabolic tumor volume. After 3 years of follow-up in ZUMA-5, axi-cel demonstrated continued durable responses, with very few relapses beyond 2 years, and manageable safety in patients with R/R iNHL.

### Introduction

Relapsed or refractory (R/R) indolent non-Hodgkin lymphoma (iNHL), including follicular lymphoma (FL) and marginal zone lymphoma (MZL), is considered largely incurable, with most patients ultimately experiencing additional disease relapses. Among patients with FL, treatment in later lines is heterogeneous, but a commonality is that remissions are progressively shorter and survival is reduced following second-line and later therapies. In addition, patients with FL who progress within 24 months from initiating the first anti-CD20-containing chemoimmunotherapy (POD24) have an unfavorable prognosis and shortened survival with available R/R treatment options.

Recent advances in therapeutic options for iNHL, including chimeric antigen receptor (CAR) T-cell therapy, have improved outcomes in patients with R/R disease. Axicabtagene ciloleucel (axi-cel) is an autologous anti-CD19 CAR T-cell therapy, which includes a CD28 co-stimulatory domain to elicit rapid and robust expansion that results in target-specific cytotoxicity and helps to overcome the limitations of the immune system. Axi-cel is approved for the treatment of adults with R/R FL. Approval was supported by the primary analysis of the ZUMA-5 trial, a single-arm, international, phase 2 study in patients with iNHL (N=104), in which the overall response rate (ORR) was 92% (74% complete response [CR] rate) after a median of 17.5 months of follow-up.

Long-term follow-up analyses are particularly important in R/R indolent lymphomas due to the heterogeneity of pre-treatment tumor characteristics and its long clinical course. Here, we report efficacy, safety, and biomarker assessments from ZUMA-5 after 3 years of follow-up, representing the longest follow-up analysis of a CAR T-cell therapy in iNHL to date. This analysis includes exploratory assessments of the association of clinical outcomes with baseline variables, including prior bendamustine exposure and baseline tumor burden as assessed by metabolic tumor volume (MTV).

### Methods

### Patients and Study Design

ZUMA-5 is a multicenter, single-arm, registrational, phase 2 trial at 17 medical centers in the USA and France and is registered at Clinicaltrials.gov/NCT03105336. A full list of sites was reported previously. Enrolled patients provided written informed consent for participation and the study protocol was approved by the institutional review board at each site.

Full patient eligibility criteria have been previously reported. Briefly, patients aged ≥18 years with R/R iNHL, including FL (grade 1-3a) and MZL (nodal or extranodal; both per WHO 2016 criteria) had ≥2 prior systemic therapies that must have included an anti-CD20 monoclonal antibody combined with an alkylating agent. Patients who had previous autologous stem cell transplantation (SCT) within 6 weeks of axi-cel, any allogeneic SCT, CD19-targeted therapy, or CAR T-cell therapy were excluded. Disease progression <6 months of completion of the most recent prior therapy was considered refractory.

### Procedures and Endpoints

Enrolled patients underwent leukapheresis, followed by lymphodepleting chemotherapy with fludarabine (30 mg/m²/day) and cyclophosphamide (500 mg/m²/day) on days −5 to −3 prior to infusion, and axi-cel (2×10⁶ CAR T cells/kg).⁹ Bridging therapy prior to lymphodepletion was per investigator discretion. Disease response assessments, as detailed previously, were performed by investigators and reviewed by an independent radiology review committee per Lugano classification (detailed previously) at specified timepoints until the 24-month follow-up analysis, after which assessments were per investigator only. All adverse events (AEs) were monitored up to month 3 postinfusion, then only AEs of special interest (neurological, hematological, infectious, and autoimmune) were monitored up to month 24; new and secondary malignancies were monitored up to 15 years.

The primary endpoint of ZUMA-5 was ORR. Secondary endpoints included CR, duration of response (DOR), progression-free survival (PFS), overall survival (OS), time to next treatment (TTNT), safety, and blood levels of CAR T cells. Exploratory endpoints included in this analysis were lymphoma-specific PFS and survival, where progression events or deaths related to lymphoma, axi-cel, or lymphodepleting chemotherapy as assessed by the investigator were considered events of interest. Clinical and pharmacokinetic outcomes were also assessed by key patient and clinical subgroups, including prior bendamustine use before leukapheresis and baseline MTV.

### Statistical Analyses

The 3-year analysis of ZUMA-5 occurred when enrolled patients with FL had median follow-up of ≥36 months. Efficacy outcomes were assessed in all enrolled patients with iNHL (intent-to-treat); safety and translational assessments were in treated patients with iNHL (laboratory and biomarker assessments were previously described). Patients with FL who had ≥3 lines of therapy, excluding those with alternate histology on baseline central assessment, were assessed in a separate analysis. Patients retreated with axi-cel were also assessed separately (retreatment criteria were previously reported).

Descriptive statistics were used to summarize baseline characteristics, response, and incidence of AEs. Two-sided 95% CIs for response rates were assessed using the Clopper-Pearson method. Secondary endpoints involving time to event outcomes were assessed using Kaplan-Meier methodology. Lymphoma-specific PFS and survival were assessed using a competing risk approach where events of interest were considered as main events while deaths not attributed to lymphoma, axi-cel, or lymphodepleting chemotherapy were considered competing risks. Event rates over time were calculated for both main events and competing risks via the cumulative incidence function.

Propensity score matching (PSM) was performed to assess outcomes in patients with FL by prior bendamustine use, accounting for the distribution of baseline MTV, ECOG score, FLIPI score, number of prior chemotherapies, age, double refractory status, and whether the last systemic therapy was administered <12 months of leukapheresis. Wilcoxon rank-sum tests were used to assess associations between CAR T-cell levels and clinical outcomes.

### Results

### Patients

A total of 159 patients were enrolled (127 with FL, 31 with MZL, 1 with DLBCL) and underwent leukapheresis, including 6 additional patients with MZL who were enrolled after the data cutoff date for the 18-month analysis. Axi-cel was successfully manufactured for all enrolled patients. In addition to untreated patients previously described, one patient had disease transformation and 1 had no measurable disease. The patient determined to have DLBCL did not receive axi-cel and discontinued the study. A total of 152 patients received conditioning chemotherapy and axi-cel as of the data cutoff date of Mar. 31, 2022 (124 with FL and 28 with MZL).

Baseline characteristics among all 159 enrolled patients are not shown. The median age was 60 years in patients with FL (range, 34-79) and 64 years in those with MZL (range, 43-77). In patients with FL, 56% had POD24 and 69% had prior bendamustine use. Baseline characteristics of patients with FL by prior bendamustine exposure before and after PSM are shown in Table 31.

TABLE 31
Baseline Characteristics in Patients With Follicular Lymphoma by
Prior Bendamustine Use Before and After Propensity Score Matching

| | Before Matching | | | | After Matching* | | |
|---|---|---|---|---|---|---|---|
| Bendamustine Use Prior to | ≤12 Months | | None | | ≤12 Months | | None |
| Leukapheresis | (n = 17) | | (n = 38) | | (n = 15) | | (n = 15) |
| MTV, median (IQR), mL | 799.3 (212.3- | | 359.3 (220.8- | | 981.7 (212.6- | | 553.2 (247.1- |
| | 2007.4) | | 777.6) | | 2089.0) | | 1114.5) |
| Age, median (IQR), years | 54 (39- | | 57 (52- | | 57 (50- | | 54 (50- |
| | 59) | | 62) | | 60) | | 63) |
| ECOG PS = 1, n (%) | 6 | (35.3) | 9 | (23.7) | 6 | (40.0) | 3 (20.0) |
| FLIPI ≥3, n (%) | 10 | (58.8) | 11 | (28.9) | 10 | (66.7) | 7 (46.7) |
| Number of prior lines of | | | | | | | |
| chemotherapy, n (%)^{†} | | | | | | | |
| ≤2 | 6 | (35.3) | 21 | (55.3) | 5 | (33.3) | 8 (53.3) |
| 3 | 6 | (35.3) | 9 | (23.7) | 5 | (33.3) | 4 (26.7) |
| ≥4 | 5 | (29.4) | 7 | (18.4) | 5 | (33.3) | 3 (20.0) |
| Double refractory to prior | 14 | (82.4) | 12 | (31.6) | 12 | (80.0) | 9 (60.0) |
| anti-CD20 mAb and | | | | | | | |
| alkylating agent, n (%) | | | | | | | |
| last systemic therapy | 17 | (100.0) | 24 | (63.2) | 15 | (100.0) | 15 (100.0) |
| administered within 12 | | | | | | | |
| months of enrollment, n (%) | | | | | | | |
| POD24 from initiating first | 13 | (76.5) | 19 | (50.0) | 11 | (73.3) | 8 (53.3) |
| anti-CD20 | | | | | | | |
| mAb-containing therapy, n (%)^{†} | | | | | | | |

*Propensity score matching was performed after 1:1 matching with log2 MTV Calipers = 1 and age Calipers = 1.5, with exact statement on PTR12MFL.
^{†}1 observation for number of prior lines of therapy was missing before and after matching in patients exposed to bendamustine ≤12 months prior to leukapheresis. 8 observations were missing for POD24 before and after matching in patients with no prior bendamustine exposure. 2 observations were missing for POD24 before and after matching in patients exposed to bendamustine ≤12 months prior to leukapheresis.
ECOG PS, Eastern Cooperative Oncology Group performance status; FLIPI, Follicular Lymphoma International Prognostic Index; IQR, interquartile range; mAb, monoclonal antibody; MTV, metabolic tumor volume; POD24, progression of disease <24 months from initiating the first anti-CD20-containing.

Baseline MTV in patients with FL positively correlated with tumor burden as measured by sum of product diameters (SPD; not shown), FLIPI score, and tumor bulk by GELF criteria, though it was not correlated with baseline lactate dehydrogenase levels.

### Efficacy in Patients With Follicular Lymphoma

Median follow-up from leukapheresis in enrolled patients with FL was 41.7 months (range, 32.7-57.4). The investigator-assessed response among enrolled patients with FL was consistent with prior analyses (ORR, 94% [95% CI, 88-97]; CR rate, 79%). Median DOR in patients with FL was 38.6 months. Median DOR was not reached in patients with a CR and was 4.9 months in those with a partial response. At data cutoff, 53% of enrolled patients with FL (67/127) were in ongoing response. Among those who achieved a CR (n=100), 65% were in ongoing response at data cutoff. Consistent with prior reporting, all 13 patients with FL retreated with axi-cel responded (69% CR; 31% PR). With a median of 23 months after retreatment, the median post-retreatment DOR was 5.0 months, with 46% of patients in ongoing response at data cutoff.

Median PFS in enrolled patients with FL was 40.2 months, with an estimated 36-month PFS rate of 54%. A total of 2 events of progression and 10 deaths occurred >24 months after leukapheresis. In a competing risk analysis of lymphoma-specific PFS in patients with FL, a total of 40 progression or death events due to lymphoma, lymphodepleting chemotherapy, or axi-cel occurred (31%), 38 of which were progression events. The 36-month cumulative incidence rate of lymphoma-specific progression or death was 34%. Competing risks (deaths exclusive of progression or study treatment) occurred in a total of 13 patients (10%) with most occurring after the 24-month time point. The cumulative incidence of competing risks at 36 months was 12%. The median PFS among patients with (n=70) or without (n=41) POD24 was 40.2 months and not reached, respectively. The estimated 36-month PFS rate was largely consistent in patients with FL, regardless of other high-risk baseline characteristics.

Patients with FL who received prior bendamustine had a numerically lower 36-month PFS rate compared with those who did not receive bendamustine; patients who received bendamustine ≤12 months of leukapheresis had notably numerically shorter PFS, though a small number of patients in this group may limit comparison (Table 32; Table 33). Further examination of prior bendamustine use after PSM demonstrated numerically higher CR and ongoing response at 36 months in patients without prior exposure to bendamustine compared with those with exposure ≤12 months of leukapheresis (Table 34). Of note, these findings were not statistically significant and these analyses were hypothesis-generating and possibly underpowered.

Table 32 shows Progression-Free Survival in Patients with Follicular Lymphoma by Timepoint of Bendamustine Use Prior to Axi-Cel Infusion. Kaplan-Meier estimates of progression-free survival among enrolled patients with FL by investigator assessment in those who had no prior bendamustine exposure (“None”), received bendamustine within 6-months of leukapheresis (“≤6 months”), received bendamustine between 6 and 12 months of leukapheresis (“6-12 Months”), and received bendamustine greater than 12 months prior to leukapheresis (“>12 months”). Axi-cel, axicabtagene ciloleucel; FL, follicular lymphoma; NE, not estimable; PFS, progression-free survival.

TABLE 32
PFS in Patients With Follicular Lymphoma by Timepoint
of Bendamustine Use Prior to Leukapheresis.

| | Benadmustine prior to Leukapheresis | | | |
|---|---|---|---|---|
| | ≤6 months | 6-12 Months | >12 Months | None |
| Estimated PFS | (n = 8) | (n = 10) | (n = 70) | (n = 39) |
| Median (95% | 6.4 (1.1- | NR (2.3- | 40.2 (24.6- | NR (35.5- |
| CI), months | 38.6) | NE) | NE) | NE) |
| 36-month rate | 25 (4- | 50 (18- | 50 (37- | 70 (50- |
| (95% CI), % | 56) | 75) | 63) | 83) |

TABLE 33
DOR, PFS, and OS in Patients With Follicular Lymphoma in the 3-Year
Analysis by Timepoint of Bendamustine Use Prior to Leukapheresis

| | Follicular Lymphoma | | | |
|---|---|---|---|---|
| | (n = 127) | | | |
| Bendamustine | ≤6 | >6-≤12 | >12 | |
| Use Prior to | Months | Months | Months | None |
| Leukapheresis | (n = 8) | (n = 10) | (n = 70) | (n = 39) |
| Median DOR | 10.5 (2.2- | NR (2.2- | 38.6 (22.7- | NR (33.6- |
| (95% CI), mo | NE) | NE) | NE) | NE) |
| Estimate at 36 months | 40.0 (5.2- | 55.6 (20.4- | 51.8 (37.7- | 69.7 (49.3- |
| (95% CI), % | 75.3) | 80.5) | 64.2) | 83.2) |
| Median PFS | 6.4 (1.1- | NR (2.3- | 40.2 (24.6- | NR (35.5- |
| (95% CI), mo | 38.6) | NE) | NE) | NE) |
| Estimate at 36 months | 25.0 (3.7- | 50.0 (18.4- | 50.4 (36.7- | 70.0 (49.6- |
| (95% CI), % | 55.8) | 75.3) | 62.6) | 83.3) |
| Median OS | 35.5 (1.1- | NR (22.2- | NR (NE- | NR (NE- |
| (95% CI), mo | NE) | NE) | NE) | NE) |
| Estimate at 36 months | 50.0 (15.2- | 39.8 (33.4- | 70.6 (58.1- | 88.7 (72.4- |
| (95% CI), % | 77.5) | 45.8) | 79.9) | 95.6) |

CR, complete response; DOR, duration of response; NE, not estimable; NR, not reached; ORR, overall response rate; PFS, progression-free survival; OS, overall survival.

TABLE 34
Clinical and Translational Outcomes by Prior Bendamustine Use in
Patients With Follicular Lymphoma After Propensity Score Matching

| Bendamustine Use Prior to | ≤12 Months | | None | | P- |
|---|---|---|---|---|---|
| Leukapheresis | (n = 15) | | (n = 15) | | Value |
| ORR, n (%) | 12 | (85.7) | 15 | (100.0) | .2241 |
| CR | 8 | (57.1) | 12 | (80.0) | .2451 |
| Ongoing response at 36 months, n (%) | 4 | (28.6) | 9 | (60.0) | .1394 |
| Grade ≥3 neurologic events, n (%) | 1 | (6.7) | 2 | (13.3) | 1 |
| Grade ≥3 CRS, n (%) | 1 | (6.7) | 2 | (13.3) | 1 |

| | | | |
|---|---|---|---|
| Peak CAR T-cell level, median (IQR), | 17.1 (3.0- | 42.5 (5.7- | .3046 |
| cells/μL | 48.4) | 62.6) | |
| AUC CAR T-cell level, median (IQR), | 181.1 (45.3- | 507.0 [99.1- | .1485 |
| cells/μL × days | 414.4) | 745.9) | |
| Total number of CD4 cells infused, | 149.3 (70.0- | 121.1 (93.0- | .5409 |
| median (IQR), n × 10⁶ | 176.1) | 205.0) | |
| Total number of CD8 cells infused, | 131.9 (90.0- | 130.4 (92.2- | .8743 |
| median (IQR), n × 10⁶ | 183.5) | 176.0) | |
| Total number of CCR7+CD45RA+ T | 21.7 (17.4- | 44.9 (24.0- | .1781 |
| cells infused, median (IQR), n × 10⁶ | 45.5) | 89.7) | |
| IFN-γ in co-culture, median (IQR), | 2871.0 (2272.5- | 6973.0 (5348.0- | .001665 |
| pg/mL | 4360.0) | 10403.0) | |

Propensity score matching was performed after 1:1 matching with log2 MTV Calipers = 1 and age Calipers = 1.5. with exact statement on PTR12MFL.
AUC, area under the curve; CAR, chimeric antigen receptor; CR, complete response; CRS, cytokine release syndrome; IFN, interferon, IQR, interquartile range; MTV, metaboic tumor volume; ORR, overall response rate.

The median OS among enrolled patients with FL was not reached, and the estimated OS at 36 months was 76%. Median TTNT was also not reached, with a 36-month estimate of 60%. A competing risk assessment of lymphoma-specific OS showed 15 deaths due to lymphoma, lymphodepleting chemotherapy or axi-cel (12%); competing risks (deaths due to other reasons) occurred in 17 patients (13%). The 36-month cumulative incidence of lymphoma-specific death was 12% (cumulative incidence of competing risks at 36-months was 12%).

In an assessment of efficacy outcomes by baseline MTV in evaluable patients with FL (n=125), DOR and PFS were longer among patients with relatively low baseline MTV, as observed below a historical threshold of 510 mL, as well as below the study median and quartiles (Table 35, Table 36, and Table 37). Notably, estimated PFS at 36 months was 71.2% in those whose baseline MTV fell below the study median and 37.3% among those above the median. No correlation between median baseline MTV and ORR or CR was observed, likely due to the low number of nonresponders in this study. Patients with baseline MTV below the study median were also more likely than those above the median to be in ongoing response at data cutoff. Association between baseline SPD and efficacy outcomes showed similar trends as those with MTV, though none reached statistical significance.

Table 35 shows Progression-Free Survival in Patients With FL by Quartile of Baseline Metabolic Tumor Volume. Kaplan-Meier plot of progression-free survival per investigator assessment by quartile of baseline metabolic tumor volume in evaluable enrolled patients with FL. FL, follicular lymphoma; NE, not estimable; PFS, progression-free survival.

TABLE 35
PFS in Patients With FL by Quartile
of Baseline Metabolic Tumor Volume

| | Quartile of MTV | | | |
|---|---|---|---|---|
| Estimated PFS | Q1 | Q2 | Q3 | Q4 |
| Median (95% | NR (24.4- | NR (38.6- | 25.4 (7.0- | 24.2 (12.8- |
| CI), months | NE) | NE) | NE) | NE) |
| 36-month rate | 60 (39- | 82 (60- | 42 (22- | 33 (16- |
| (95% CI), % | 79) | 92) | 61) | 51) |

TABLE 36
Efficacy Outcomes in the 3-Year Analysis by Study Median
and Historical Threshold of Metabolic Tumor Volume

| | Follicular Lymphoma | | Marginal Zone Lymphoma | | |
|---|---|---|---|---|---|
| | (n = 127) | | (N = 31) | | |
| | ≤438.5 mL | >438.5 mL | ≤368.8 mL | >368.8 mL | |
| Study median | (n = 63) | (n = 62) | (n = 14) | (n = 13) | |
| ORR, n (%) | 61 (97) | 56 (90) | 12 (86) | 11 | (85) |
| CR | 55 (87) | 44 (71) | 10 (71) | 9 | (69) |
| Median DOR (95% CI), mo | NE (36.6- | 23.5 (11.8- | NE (1.9- | NE (5.1- | |
| | NE) | NE) | NE) | NE) | |
| Estimate at 36 months (95% | 73.3 (58.4- | 40.4 (26.1- | 56.3 (24.4- | NE (NE- | |
| CI), % | 83.5) | 54.3) | 79.1) | NE) | |
| Median PFS (95% CI), mo | NE (38.6- | 24.2 (13.4- | NE (5.5- | NE (7.0- | |
| | NE) | 40.2) | NE) | NE) | |
| Estimate at 36 months (95% | 71.2 (56.8- | 37.3 (23.9- | 51.9 (22.5- | 55.9 (24.0- | |
| CI), % | 81.6) | 50.7) | 75.0) | 79.0) | |
| | ≤510 mL | >510 mL | ≤510 mL | >510 mL | |
| Historical threshold | (n = 68) | (n = 57) | (n = 16) | (n = 11) | |
| ORR, n (%) | 66 (97) | 51 (89) | 14 (88) | 9 | (82) |
| CR | 57 (84) | 42 (74) | 11 (69) | 8 | (73) |

| | | | | |
|---|---|---|---|---|
| Median DOR (95% CI), mo | NE (36.6- | 24.7 (11.8- | NE (5.1- | NE (6.2- |
| | NE) | NE) | NE) | NE) |
| Estimate at 36 months (95% | 70.7 (56.6- | 40.6 (25.5- | 55.6 (26.4- | NE (NE- |
| CI), % | 81.0) | 55.1) | 77.2) | NE) |
| Median PFS (95% CI), mo | NE (38.6- | 25.2 (13.6- | NE (5.5- | NE (10.1- |
| | NE) | NE) | NE) | NE) |
| Estimate at 36 months (95% | 68.9 (55.1- | 37.1 (23.1- | 51.9 (24.5- | 56.8 (21.3- |
| CI), % | 79.2) | 51.1) | 73.6) | 81.3) |

CR, complete response; DOR, duration of response; NE, not estimable; NR, not reached; ORR, overall response rate; PFS, progression-free survival.

TABLE 37
Efficacy Outcomes in Patients With Follicular Lymphoma in
the 3-Year Analysis by Quartiles of Metabolic Tumor Volume

| | Follicular Lymphoma | | | |
|---|---|---|---|---|
| | (n = 127) | | | |
| | Min-Q1 | Q1-Median | Median-Q3 | Q3-Max |
| | (11.2- | (183.9- | (438.5- | (1076.0- |
| Quartile of MTV, mL | 183.9) | 438.5) | 1076.0) | 5576.6) |
| ORR, n/n (%) | 30/32 | 31/31 | 27/28 | 29/30 |
| | (94) | (100) | (96) | (97) |
| CR | 26/32 | 29/31 | 20/28 | 24/30 |
| | (81) | (94) | (71) | (80) |
| Median DOR | NR (22.7- | NR (36.6- | 29.0 (10.4- | 22.0 (11.5- |
| (95% CI), mo | NE) | NE) | NE) | NE) |
| Estimate at 36 | 63.3 (40.1- | 81.5 (60.1- | 45.1 (23.9- | 36.2 (17.7- |
| months (95% CI), % | 79.6) | 92.1) | 64.1) | 55.2) |
| Median PFS | NR (24.4- | NR (38.6- | 25.4 (7.0- | 24.2 (12.8- |
| (95% CI), mo | NE) | NE) | NE) | NE) |
| Estimate at 36 | 60.4 (39.0- | 81.5 (60.1- | 41.9 (22.2- | 33.0 (15.8- |
| months (95% CI), % | 76.3) | 92.1) | 60.5) | 51.4) |

CR, complete response; DOR, duration of response; NE, not estimable; NR, not reached; ORR, overall response rate; PFS, progression-free survival.

Efficacy results are reported separately in a subset of patients with FL with ≥3 prior lines of therapy after excluding patients whose central pathology assessment suggested alternate diagnoses other than FL. The outcomes were largely consistent with the overall cohort.

Efficacy in Patients with Marginal Zone Lymphoma

Median follow-up in enrolled patients with MZL from leukapheresis was 31.8 months (range, 8.3-52.3). The investigator-assessed ORR in enrolled patients with MZL was 77% (95% CL, 59-90), with a CR rate of 65%. Responses among subtypes of MZL (nodal and extranodal) are not shown. Median DOR in all patients with MZL was not yet reached, and 52% of patients 16/31) were in ongoing response as of data cutoff.

Median PFS, OS and TTNT were not yet reached among patients with MZL, and estimates at 24-month were 56%, 74%, and 53%, respectively. PFS estimates at 24 months largely consistent among high-risk subgroups. No correlations were observed between baseline MTV and efficacy outcomes among patients with MZL, possibly due to the small number of patients with this disease type on study (Table 36 above).

### Safety

No new safety signals were observed among treated patients with iNHL since the 18-month analysis. AEs that occurred after the 18-month analysis (data cutoff date, Sep. 14, 2020) were largely in recently enrolled patients with MZL, including 1 grade 3 neurologic event, 2 infections of grade 3-4, and 5 cytopenias of grade 3-4. Serious AEs occurred in 15 patients (10%; 11 with FL and 4 with MZL) since the 18-month analysis; events in 6 of those patients were considered related to axi-cel (3 in FL and 3 in MZL). No new cases of grade≥3 hypogammaglobulinemia occurred after the data cutoff date for the primary analysis (Mar. 12, 2020). Additionally, there were no new secondary malignancies since the 18-month analysis. During the course of the study, a total of 50 patients with iNHL (33%) received immunoglobulin therapy. No cases of axi-cel-related secondary malignancies, tumor lysis syndrome, or replication-competent retrovirus occurred at any time on study.

When assessing all events on study in iNHL, grade≥3 cases of cytokine release syndrome appeared to occur more frequently among patients aged ≥65 years (12%) than those <65 years (4%). Similarly, grade≥3 neurologic events occurred numerically more frequently in those ≥65 years (27%) than those <65 years (14%). Among patients with iNHL who had any grade 3 cytopenias on or after day 30 postinfusion (n=51), 5 had cytopenias present 12-months postinfusion and 4 had cytopenias 24-months post-infusion. No correlations were observed between baseline MTV and either grade≥3 cytokine release syndrome or neurologic events, possibly due to the low incidence of grade≥3 toxicities. In total, 39 deaths occurred in ZUMA-5, 14 of which were lymphoma-related as assessed by investigators (11 due to complications of underlying lymphoma and 3 due to AEs related to study treatment in patients with FL). Safety results in the subset of patients with FL with ≥3 prior lines of therapy excluding those with suggested alternative diagnosis are not shown.

### Biomarkers

Among treated patients with iNHL, median peak CAR T-cell levels were significantly higher in those with ongoing responses at 36 months (53.9 cells/μL) than those who relapsed (29.6 cells/μL) or nonresponders (22.2 cells/μL). Most treated patients with FL had detectable B cells by Month 12. By Month 24, half of patients with ongoing response had low levels of detectable CAR gene-marked cells. The levels of CAR gene-marked cells were inversely correlated with that of B cells at each timepoint post-infusion.

Among 14 patients with iNHL (13 FL; 1 MZL) with evaluable tumor biopsies at progression, all patients had detectable B-cell antigens, CD19 and CD20. Though PFS was similar among patients with POD24 and those without, those with POD24 had higher pre-treatment levels of macrophage-associated chemokines, CCL17 (TARC) and CCL22 (MDC) compared with patients without POD24. These analytes have been previously associated with relapse in patients with FL.

Treated patients with FL who received any prior bendamustine treatment appeared to have lower CAR T-cell expansion by peak and area under the curve (AUC), along with a lower proportion of naive (CCR7+CD45RA+) T cells in axi-cel product, versus those without bendamustine exposure (Table 38).

TABLE 38
CAR T-cell Expansion and Axi-Cel Product Characteristics in Treated Patients
with Follicular Lymphoma by the Timeline of Prior Bendamustine Use

| | | ≥6 months and | | |
|---|---|---|---|---|
| Bendamustine exposure | <6 months | ≤12 months | >12 months | None |
| prior to Leukapheresis | (n = 7) | (n = 10) | (n = 69) | (n = 38) |
| Median CAR T-cell Expansion | | | | |
| (range) | | | | |
| Peak, cells/μL | 35.4 (3.34- | 18.8 (2.7- | 32.9 (11.3- | 60.2 (23.7- |
| | 112.8) | 53.9) | 112.1) | 182.5) |
| AUC₀₋₂₈, cells/μL × days | 337.6 (53.2- | 236.9 (37.4- | 404.5 (143.6- | 614.4 (303.2- |
| | 410.9) | 527.2) | 1037.2) | 1918.6) |
| Product Characteristics | | | | |
| (range) | | | | |
| Total no. T cells infused × 10⁶, n | | | | |
| CCR7+CD45RA+ T cells, % | 6.6 (4.9- | 11.9 (7.0- | 14.9 (10.1- | 26.8 (17.4- |
| | 46.5) | 16.0) | 25.8) | 36.9) |
| CCR7+CD45RA− T cells, % | 15.8 (6.1- | 11.4 (6.7- | 19.6 (11.8- | 22.9 (14.2- |
| | 21.0) | 26.9) | 24.6) | 28.9) |
| CCR7−CD45RA+/− T cells, % | 74.9 (35.2- | 71.4 (50.2- | 62.5 (48.2- | 52.0 (32.2- |
| | 89.0) | 79.8) | 72.7) | 59.4) |

In the PSM analysis, those with bendamustine exposure ≤12 months prior to leukapheresis demonstrated numerically lower CAR T-cell expansion and number of infused CCR7+CD45RA+ T cells compared with those with no bendamustine exposure, though small numbers of patients in the analysis limit comparison. Additionally, interferon-7 in co-culture was significantly higher in those with no prior exposure to bendamustine (Table 34 above).

### Discussion

This analysis of ZUMA-5 represents the longest follow-up of a registrational trial of an anti-CD19 CAR T-cell therapy in patients with iNHL. With over 3 years of follow-up in patients with FL, axi-cel demonstrated durable remissions in a substantial proportion of patients, with more than half of patients in ongoing response as of data cutoff. Durability of response appeared to be associated with best response, as demonstrated in aggressive lymphomas. Additionally, patients with MZL appeared to have improved PFS (not yet reached) with longer follow-up than in the prior analysis. These findings represent a considerable advancement in clinical outcomes for R/R indolent lymphomas, for whom standard noncellular therapies provide limited durable remission off therapy.

After a median follow-up of 41.7 months in ZUMA-5, the median PFS in patients with FL was over 3 years (40.2 months), comparing favorably to the bispecific antibody mosunetuzumab (median PFS, 17.9 months), recently approved in patients with FL in the third-line, though additional follow-up is needed to determine long-term survival for this class of therapy and there are inherent limitations in comparing outcomes across different trials. However, corroborating these findings, the retrospective analysis comparing findings in ZUMA-5 to other standard treatments for FL (SCHOLAR-5) after 2 years of follow-up, demonstrated significant benefit in PFS with axi-cel compared with the standardized mortality-weighted control cohort (39.6 months vs 12.7 months, hazard ratio 0.28). Median OS was not yet reached in patients with FL in the current study, even though the majority of patients received ≥3 prior lines of therapy (exclusive of single agent anti-CD20 antibody), an indicator of poor prognosis in the modern era. Additionally, over half of patients had POD24 disease, and PFS in these high-risk patients appeared largely similar to those without POD24. Similar to axi-cel, tisagenlecleucel appeared to have favorable survival in R/R FL, with PFS and OS not yet reached after 16.6 months of follow-up in the ELARA study, further supporting the durability of CAR T-cell therapy in FL. Late progression events or deaths related to axi-cel or lymphodepleting therapy were uncommon. Indeed, competing risk analysis of lymphoma-specific PFS suggested that most events (83%) occurring after the 24-month time point were due to competing risks and there is emergence of a plateau beyond 2 years when considering lymphoma-specific PFS. However, longer follow-up will be needed to determine the curative potential of axi-cel in FL.

Bendamustine, a conventional treatment for both aggressive and indolent lymphomas, may attenuate T-cell fitness, and has been shown to affect CAR T-cell expansion and thus its efficacy. Patients in ZUMA-5 with FL who had exposure to bendamustine ≤12 months of leukapheresis had shorter PFS after axi-cel, supporting other recent findings with brexucabtagene autoleucel in mantle cell lymphoma. This effect was sustained after PSM that accounted for administration of systemic therapy ≤12 months before leukapheresis along with other key prognostic factors and baseline characteristics. CAR T-cell expansion was less robust and CCR7+CD45RA+ T cells in axi-cel product were numerically lower with recent bendamustine exposure, correlates of efficacy with axi-cel in FL. Taken together, these results suggest that treatment with bendamustine-based therapy may be carefully considered in patients who are likely to need CAR T-cell therapy in the future, especially high-risk patients such as those with POD24 if they were not previously exposed to bendamustine. Though, given the preliminary, hypothesis-generating nature of this analysis and lack of comparitor, further assessments are needed to determine whether and to what extent the inferior clinical outcomes in this subset of patients were due to the unfavorable underlying disease or prior exposure to bendamustine.

Baseline MTV that was above the median correlated negatively with efficacy outcomes including DOR and PFS in patients with FL. These findings corroborate similar outcomes differentiated by baseline MTV among patients with aggressive lymphomas in the ZUMA-7 randomized controlled trial. Similar to this analysis, findings from the ZUMA-7 trial showed that baseline SPD was not predictive of event-free survival, while baseline MTV was, suggesting MTV was a more accurate measure of baseline tumor burden. Of note, median baseline MTV was much higher in ZUMA-5 than that in the ELARA study (438.5 mL versus 155.3 mL). While outcomes were numerically inferior in patients in ZUMA-5 with relatively high baseline MTV, the majority of them achieved a CR (71%) and median DOR was approximately 2 years. In contrast, the CR rate in ELARA among patients with high baseline MTV was 40%. Additional correlative studies on tumor biology and T cell fitness are needed in the future to better understand the reasons for inferior outcomes in patients with high tumor burden.

Durable responses emerged in patients with MZL in this long-term follow-up analysis with enrollment of approximately 25% more patients since the prior analysis and more mature follow-up. Patients with MZL in ZUMA-5 had PFS and OS not yet reached after 31.8 months median follow-up. PFS compared favorably with those with second-line ibrutinib in the phase 2 PCYC-1121 trial in R/R MZL with similar follow-up (median PFS 15.7 months), though OS was also not reached in this trial. Continued follow-up to determine long-term durability in this population is needed.

No new safety signals were observed among treated patients with both FL and MZL since the prior analysis. AEs that emerged in this analysis were those in recently enrolled patients, and as in the primary analysis, events were generally of low grade and reversible. Notably, very few patients had prolonged high-grade cytopenias. Consistent with the prior analysis, durability of response correlated with early CAR T-cell expansion, and functional CAR T-cell persistence did not appear to be required for such durability in patients with FL. Though baseline biomarkers associated with relapse were elevated among patients with POD24, survival with axi-cel was not impacted by it.

In conclusion, long-term results demonstrate the continued durable clinical benefit of axi-cel among patients with indolent lymphomas and a manageable long-term safety profile. A substantial proportion of patients with R/R iNHL remained alive without progression.

### Example 5

This example is related to the impact of debulking therapy on the clinical outcomes of axicabtagene ciloleucel in the treatment of relapsed or refractory large B-cell lymphoma.

As disclosed in this example, embodiments of the disclosure relate to methods of treating a subject with lymphoma comprising administering to the subject a debulking regimen; and administering to the subject an immunotherapy following the debulking regimen.

In some embodiments, the debulking regimen comprises any of the options included in Table 39. In some embodiments, the debulking regimen comprises at least 2 of the options included in Table 39.

In some embodiments, the immunotherapy comprises an anti-CD19 CAR T-cell.

In some embodiments, the subject is administered the debulking regimen when the subject has a tumor burden above a predetermined level. In some embodiments, the subject is not administered the debulking regimen when the subject has a tumor burden below a predetermined level.

Axicabtagene ciloleucel (axi-cel), an autologous anti-CD19 chimeric antigen receptor T-cell therapy, was approved for relapsed/refractory (R/R) large B-cell lymphoma (LBCL) based on the results from pivotal Cohorts 1+2 of ZUMA-1 (NCT02348216). ZUMA-1 was expanded to investigate safety management strategies aimed at reducing the incidence and severity of cytokine release syndrome (CRS) and neurologic events (NEs). Prospective safety expansion Cohort 5 evaluated the impact of debulking therapy, including rituximab-containing immunochemotherapy regimens and radiotherapy, in axi-cel-treated patients; the CRS and NE management strategy paralleled those in Cohorts 1+2. Among the 50 patients in Cohort 5 who received axi-cel, 40% received ≥3 prior lines of chemotherapy, and 40% had disease that progressed while on the most recent chemotherapy. Forty-eight patients (96%) received debulking therapy, 14 (28%) radiotherapy only, and 34 (71%) systemic immunochemotherapy. Median decrease in tumor burden (per sum of product of diameters of target lesions) relative to screening was 17.4% with R-ICE/R-GDP, 4.3% with other debulking chemotherapies, and 6.3% with radiotherapy only. All patients were followed for ≥8 months. CRS was reported in 43 patients (86%), with 1 patient (2%) experiencing grade≥3. NEs were reported in 28 patients (56%), with 6 (12%) experiencing grade≥3. Cytopenias were the most frequent grade≥3 adverse event (AE); 19 (38%) and 18 (36%) treated patients had any and grade≥3 prolonged thrombocytopenia, respectively, and 25 (50%) and 24 (48%) patients had any and grade≥3 prolonged neutropenia, respectively. Overall, patients who received debulking chemotherapy had higher incidences of serious treatment-emergent AEs than those who received radiotherapy only. At the 24-month analysis, objective response rate was 72%, and complete response rate was 56%. Median duration of response, progression-free survival, and overall survival were 25.8, 3.1, and 20.6 months, respectively. These results from exploratory Cohort 5 demonstrate the feasibility of debulking prior to axi-cel, and together with current real-world evidence, suggest that debulking regimens may help minimize the frequency and severity of CRS and NEs in patients with R/R LBCL.

As discussed below, safety expansion Cohort 5 prospectively evaluated the impact of debulking therapy on the incidence and severity of cytokine release syndrome and neurologic events in patients treated with axi-cel. Here is reported the Cohort 5 primary analysis and an updated analysis with at least 2 years of follow-up.

### Methods

### Patients

Eligibility criteria for Cohort 5 were similar to the pivotal ZUMA-1 Cohorts 1+2. Patients were ≥18 years with histologically confirmed R/R LBCL after two or more lines of therapy. Refractory disease was defined as progressive disease (PD) or stable disease (SD) as the best response to the most recent therapy regimen or PD or relapse within 12 months after autologous stem cell transplantation. The study was conducted in accordance with the Good Clinical Practice guidelines of the International Conference on Harmonization and was approved by the institutional review board at each site. All patients provided informed consent before being included in the study.

### Treatment

Patients in Cohort 5 received debulking therapy after leukapheresis and prior to administration of lymphodepleting chemotherapy and axi-cel. Debulking regimens were meant to reduce lymphoma burden, and the choice of debulking therapy was made by the investigator from a list of options that included rituximab-containing immunochemotherapy regimens and radiotherapy (Table 39). Consistent with ZUMA-1 Cohorts 1+2, patients received lymphodepleting chemotherapy for 3 days (cyclophosphamide 500 mg/m²/day and fludarabine 30 mg/m²/day on Days −5, −4, and −3) prior to a single intravenous infusion of axi-cel (target dose, 2×10⁶ anti-CD19 CAR T cells/kg) on Day 0. Cohort 5 followed the safety management strategy of Cohorts 1+2, which was no prophylactic or earlier steroids; however, in contrast to Cohorts 1+2, patients in Cohort 5 received prophylactic levetiracetam (750 mg oral or intravenous twice daily) starting on Day 0 to manage potential neurologic events after axi-cel treatment.

### Endpoints

The descriptive primary endpoints were the incidence and severity of cytokine release syndrome and neurologic events. Cytokine release syndrome was defined and graded as previously published. Neurologic events were identified by a search term list per as previously published and graded for severity per Common Terminology Criteria for Adverse Events version 4.03. Secondary endpoints included investigator-assessed objective response rate (complete response and partial response) based on revised International Working Group Response Criteria for Malignant Lymphoma, duration of response, progression-free survival, overall survival, incidence of adverse events, and levels of anti-CD19 CAR T cells and cytokines in blood. CIs for objective response rates were generated by the Clopper-Pearson method. CIs and landmark estimates of duration of response, progression-free survival and overall survival were generated using the Kaplan-Meier survival method. For duration of response and progression-free survival, disease assessment after the initiation of new anticancer therapy, not including stem cell transplantation, was not included in the derivation. Exploratory endpoints included biomarker analyses.

### Statistical Analyses

Similar to other ZUMA-1 safety management cohorts, Cohort 5 was not designed for formal hypothesis testing and all analyses were descriptive. The primary analysis was conducted when all 50 patients treated were followed for ≥6 months after axi-cel infusion; in addition, an updated analysis was performed when each patient had been followed for ≥24 months. The safety analysis set included all patients treated with any dose of axi-cel, and the modified intent-to-treat (mITT) population comprised those treated with axi-cel at a target dose of at least 1×10⁶ CAR T cells/kg and was used for efficacy-based endpoints. For post hoc debulking subgroup analyses, outcomes between patients receiving chemotherapy versus radiotherapy only were assessed. In addition, chemotherapy regimens were grouped into two categories: more intensive regimens, including rituximab, ifosfamide, carboplatin, and etoposide (R-ICE) or rituximab, gemcitabine, dexamethasone, and cisplatin (R-GDP; R-ICE/R-GDP group), and other less aggressive debulking chemotherapies. Tumor burden was measured by sum of product of diameters of target lesions. Descriptive P values, calculated by Wilcoxon 2-sample test, were generated to compare pharmacokinetic parameters with toxicity severity. Exploratory, retrospective propensity score matching (PSM) analysis was performed to descriptively compare results of the primary analysis for Cohort 5 with those of Cohorts 1+2.

### Results

### Patients

Patients were enrolled in Canada (30%), France (26%), Netherlands (26%), and Germany (18%) between December 2018 and December 2019. Of the 58 patients enrolled and leukapheresed, 54 (93%) received debulking therapy, 51 (88%) received lymphodepleting chemotherapy, and 50 patients received axi-cel at the target dose. Eight enrolled patients that underwent leukapheresis did not receive axi-cel due to failure to meet eligibility criteria (n=3), adverse event related to refractory disease (n=1), withdrawn consent (n=1), and death due to disease progression (n=3). At the data cutoff for the primary (Sep. 10, 2020) and 24-month (Jan. 10, 2022) analyses, the median follow-up was 15.1 months (range, 8.0-18.8) and 31.1 months (range, 24.0-34.8), respectively. Among patients who were treated with axi-cel (mITT population), the median age was 57.5 years (range, 29-74), most patients (74%) had stage III or IV disease, 40% had received 3 or more prior lines of chemotherapy, and 40% had PD as the best response to the most recent chemotherapy (Table 40).

Forty-eight patients treated with axi-cel (96%) received debulking therapy; 34 (71%) received systemic chemotherapy and 14 (28%) received radiotherapy only. Among the patients receiving debulking chemotherapy, 17 patients (34%) received intensive chemotherapy regimens (R-ICE/R-GDP), and 17 (34%) received other less aggressive debulking chemotherapies (including R-GEMOX [rituximab, gemcitabine, and oxaliplatin] and R-CHOP [rituximab, cyclophosphamide, doxorubicin, vincristine, and prednisone]). Two patients received more than one type of debulking regimen: one received radiotherapy and R-CHOP, and one received R-GEMOX and prednisone. The median time from leukapheresis to axi-cel delivery at the study site was 31 days (range, 23-71) in Europe and 21 days (range, 18-22) in Canada. The median time from leukapheresis to axi-cel infusion was 33 days (range, 25-71) in Europe and 33 days (range, 27-51) in Canada. No significant differences in manufacturing times were observed based on type of debulking therapy.

### Safety

### Primary Analysis

In the primary analysis, all 50 patients (100%) experienced at least one treatment-emergent adverse event (TEAE), and 25 patients (50%) had at least one serious TEAE. The most common any-grade TEAEs were pyrexia (86%), hypotension (52%), neutrophil count decreased (50%), anemia (38%), headache (34%), platelet count decreased (34%), and neutropenia (32%). All 50 patients (100%) experienced grade≥3 TEAEs, the most common of which were neutrophil count decreased (48%), anemia (30%), and neutropenia (30%) (Table 41). Grade 4 adverse events were reported in 70% of patients, and 10% of patients had grade 5 adverse events. Serious TEAEs were more common among patients who received intensive chemotherapy debulking (R-ICE/R-GDP) versus other less aggressive chemotherapies (including R-GEMOX and R-CHOP) or radiotherapy only, and incidence of grade≥3 infections were higher in patients who received R-ICE/R-GDP versus other debulking therapies (Table 42).

Cytokine release syndrome was reported in 43 patients (86%), all with severity grade 1 or 2 except one patient (2%) who received ifosfamide, gemcitabine, vinorelbine, and corticosteroid debulking and experienced grade 4 cytokine release syndrome (Table 43). The most frequently reported any-grade cytokine release syndrome symptoms were pyrexia (n=41, 95%), hypotension (n=24, 56%), chills (n=10, 23%), and nausea (n=9, 21%). The median time to cytokine release syndrome onset was 2 days (range, 1-9 days) after axi-cel infusion and among the 42 patients whose cytokine release syndrome resolved, the median duration was eight days (range, 1-19). One patient had grade 4 hypoxia reported on Day 17 and grade 2 tachycardia reported on Day 31 that were ongoing at the time of death due to grade 5 pneumonia influenza type A (related to lymphodepleting chemotherapy) on Day 42. Any-grade cytokine release syndrome was more common among patients who received intensive chemotherapy debulking (R-ICE/R-GDP; 17/17 patients [100%]) and radiotherapy only (14/14 patients [100%]) versus other less aggressive debulking chemotherapy (11/17 patients [65%]; Table 42).

The overall incidence of neurologic events was 56% (n=28), with 12% (n=6) of patients experiencing grade≥3 events (Table 43). The most frequent any-grade neurologic events were tremor (n=14, 28%), aphasia (n=9, 18%), and confusional state (n=8, 16%). The median time to neurologic event onset was eight days (range, 1-17) after axi-cel infusion, and among the 23 patients whose neurologic events resolved, the median duration was 12 days (range, 1-99). At primary data cutoff, five patients had unresolved neurologic events, three of whom had died (n=1 each due to progressive disease, septic shock [related to axi-cel], and pneumonia influenza type A [aforementioned]). Any-grade neurologic events occurred at similar incidence across debulking groups (Table 42).

Overall, 26 (52%) patients received corticosteroids for the management of cytokine release syndrome (14 patients; 28%), neurologic events (14 patients; 28%), and/or other reasons (10 patients; 20%). The median cumulative cortisone-equivalent corticosteroid dose received was 3599.5 mg (range, 125.2-138, 725.2). Of the 26 patients who received corticosteroids, 25 also received tocilizumab. Thirty-nine patients (78%) received tocilizumab for the treatment of cytokine release syndrome (37 patients; 74%) and/or neurologic events (5 patients; 10%).

Infections occurred in 19 patients (38%), including four patients (8%) with grade 3 infections and four patients (8%) with grade 5 infections (not shown). Grade 5 infections included three patients (6%) with septic shock, reported on Days 27, 40, and 144, and one (2%) with pneumonia influenza type A, related to lymphodepleting chemotherapy reported on Day 42. One patient (2%) developed COVID-19 (grade 3). The median time to onset of infection was 10 days (range, 2-282). In general, any-grade infections were more common among patients who received debulking radiotherapy versus debulking chemotherapy regimens, with grade≥3 events most common among those who received intensive chemotherapy regimens (R-ICE/R-GDP; Table 44). Hypogammaglobulinemia was reported in four patients (8%); all events were grade 1 or 2 (not shown). Intravenous immunoglobulin therapy was administered to three patients (6%), around 1 month after axi-cel infusion in all 3 cases. The incidence of grade≥3 prolonged cytopenias (i.e., present on or after Day 30 following axi-cel infusion) was 52% (n=26), the most common being neutropenia (n=24, 48%), followed by thrombocytopenia (n=18, 36%), and anemia (n=7, 14%).

New malignancies were reported in two patients. Both patients developed grade 3 myelodysplastic syndrome (MDS), one on Day 363 and the other on Day 496 (evolved to grade 5 on Day 884), that were considered related to lymphodepleting chemotherapy per investigator assessment.

A total of 23 of 50 treated patients (46%) died during the primary analysis period. TEAE-related deaths were the four grade 5 infections noted above. Of the remaining 19 deaths, 17 were due to progressive disease, one due to respiratory failure (in the setting of disease progression), and one due to sepsis that was secondary to lymphoma.

### Updated Analysis: 24-Month Follow Up

The 24-month safety results were similar to those of the primary analysis. Seven serious TEAEs were reported in three patients after the primary analysis, including 2 new malignancies. The first patient experienced 5 events, including pyrexia (grade 1) and neutropenia (grade 3) on Day 272, cellulitis (grade 3) on Day 275, sepsis (grade 4) on Day 548, and MDS (grade 4; related to lymphodepleting chemotherapy) on Day 485 that evolved to grade 5 on Day 552. The second patient experienced pneumonia (grade 3) on Day 559. Finally, the third patient reported a new malignancy of acute myeloid leukemia on Day 668 which was ongoing at the 24-month data cutoff date. None of these TEAEs were considered related to axi-cel treatment per investigator assessment, except the case of neutropenia.

Overall, five deaths occurred between the primary and 24-month data cutoffs: two from progressive disease, two from MDS (both aforementioned, one in primary analysis and one in the updated 24-month analysis), and one from ischemic intestine followed by septic shock, none of which were deemed related to axi-cel treatment.

No additional cases of cytokine release syndrome or neurologic events were reported after the primary analysis. Of the two patients who were alive with unresolved neurologic events at primary data cutoff, one had grade 2 lethargy and died on Day 552 (due to development of MDS) and the other was alive at the 24-month data cutoff with unresolved grade 1 amnesia. Neither neurologic event was related to any study treatment per investigator assessment. The incidence of any-grade infection was the same at 24 months as the primary analysis, though grade≥3 infections were increased by one patient (grade 4 sepsis reported on Day 548 and resolved on Day 552). No additional intravenous immunoglobulin therapy was administered after the primary analysis. B cells were detectable in two of 16 evaluable patients (13%) at Month 3 after axi-cel infusion and in five of 12 evaluable patients (42%) at 24 months (not shown). Anti-axi-cel antibodies were not detected, and no case of replication-competent retrovirus was reported.

### Efficacy

### Primary Analysis

Among the 48 patients who received debulking therapy, the median tumor burden was reduced from 2058.0 mm² at screening to 1390.0 mm² at postdebulking baseline. Median decrease in tumor burden relative to screening was 17.4% with intensive debulking chemotherapy regiments (R-ICE/R-GDP; from 3136.0 mm² at screening to 1896 mm² at postdebulking baseline), 4.3% with other less aggressive debulking chemotherapies (from 1452.0 mm² to 980.0 mm²), and 6.3% with radiotherapy only (1932.0 mm² to 1652.0 mm²). At primary data cutoff, the objective response rate was 72% (95% CI, 58%-84%), with a complete response rate of 54% (95% CI, 39%-68%). At a median follow-up of 11.4 months, the median duration of response was not reached (95% CI, 2.2 months, not estimable), with 21 of 36 patients (58%) in ongoing response; 21 patients (42%) remained in ongoing response at data cutoff. Median progression-free survival and overall survival were 3.1 months (95% CI, 2.9 months—not estimable) and 14.6 months (95% CI, 12.5 months—not estimable), respectively. Efficacy outcomes appeared improved for patients who received debulking chemotherapy regimens (R-ICE/R-GDP or other less aggressive debulking chemotherapies) versus those who received debulking by radiotherapy only; although, results should be interpreted with caution due to the small number of patients included in the different groups. ORR were 76%, 71% and 64% for patients treated with intensive chemotherapy regimens (R-ICE/R-GDP), other less aggressive debulking chemotherapies, and radiotherapy only, respectively. CR rates were 71%, 53%, and 36% for the same groups. The 6-month progression-free survival estimates were 53 months for both chemotherapy groups and 21 months for the radiotherapy-only group. Median overall survival was not reached (95% CI, 4.7—not estimable), 14.6 months (95% CI, 12.5—not estimable), and 11.6 (95% CI, 4.6—not estimable) for patients treated with R-ICE/R-GDP, other debulking chemotherapies, and radiotherapy only, respectively. Two patients achieved complete response after debulking and went on to receive axi-cel; both patients remained in CR until last assessment on study.

### Updated Analysis: 24-Month Follow Up

At the 24-month analysis, the objective response rate was unchanged from the primary analysis; as one patient converted from partial response to complete response at the Month 30 visit (on Day 908), the complete response rate increased to 56%. Median duration of response was 25.8 months (95% CI, 2.2 months—not estimable), and 18 patients (36%) were in ongoing response at time of data cutoff. Median progression-free survival was 3.1 months (95% CI, 2.9-29.1) and median overall survival was 20.6 months (95% CI, 12.6 months—not estimable).

### Translational Analyses

### Primary Analysis

The following is a summary of pre-infusion immunotherapy product characteristics. The median number of infused T cells was 277.7×10⁶ cells (range, 161.3-941.2) and the median number of infused CAR T cells was 160.0×10⁶ cells (range, 80.0-200.0). Of these, the median percentage of viable cells was 94.0% (range, 82.0-97.0). The median peak levels and AUC₀₋₂₈ of anti-CD19 CAR T cells were 26.63 cells/μL (range, 0.05-692.89) and 184.75 cells/μL×days (range, 0.16-4613.91), respectively. At 24 months, the median level of anti-CD19 CAR T cells in the blood was 0.13 cells/μL (range, 0-0.65).

A potential association between anti-CD19 CAR T-cell peak with frequency of grade≥2 cytokine release syndrome and grade≥3 neurologic events was observed. Median peak level was higher for patients with grade≥2 cytokine release syndrome compared with patients with grade 1 cytokine release syndrome (52.18 vs 17.48 cells/μL; descriptive P=0.0143), and peak level was also higher for patients with grade≥3 neurologic events compared with patients with grade ≤2 neurologic events (135.84 vs 24.29 cells/μL; descriptive P=0.2035). These results should be interpreted with caution due to the small number of patients with grade≥3 neurologic events. The median time-to-peak for 18 preselected serum analytes was between six and eight days after axi-cel infusion, except for granulocyte-macrophage colony-stimulating factor (GM-CSF) (3 days), interleukin (IL)-15 (4 days), IL-2 (5 days), IL-7 (4 days), and perforin (29 days). With the exception of intercellular adhesion molecule 1 (ICAM-1), perforin, vascular cell adhesion protein 1 (VCAM-1), and GM-CSF, analytes were elevated by at least 2-fold at peak compared with baseline in at least 50% of patients.

### Updated Analysis: 24-Month Follow Up

Peak CAR T-cell levels in the blood were numerically higher among patients in ongoing response or among nonresponders at 24 months versus those who relapsed by the data cutoff date; these differences were not significant, possibly due to the small number of patients. Moreover, peak pharmacokinetic expansion did not appear to be altered by debulking compared with ZUMA-1 Cohorts 1+2.

### Propensity Score Matching

After PSM, patient characteristics at baseline were balanced between Cohorts 1+2 and Cohort 5, with variations within 0.2 standardized mean difference. Of the 50 patients in Cohort 5, nine could not be matched to patients in Cohort 1+2 because their propensity scores were outside the prespecified boundary. Although CAR T-cell peak was similar in the two groups, the AUC₀₋₂₈ was lower in Cohort 5 than Cohorts 1+2. Responses were more frequently observed in Cohorts 1+2, including objective response rate (92.7% vs 70.7%) and complete response (61.0% vs 51.2%). Grade≥3 cytokine release syndrome was more frequent in Cohorts 1+2 compared with Cohort 5 (9.8% vs 2.4%), with a similar time to onset in both groups (median onset time was 6 and 7 days for Cohort 1+2 and Cohort 5, respectively). Similarly, grade≥3 neurologic events were more frequent in Cohorts 1+2 compared with Cohort 5 (26.8% vs 14.6%), with a similar time to onset in both groups (median onset time was 7 and 7.5 days for Cohort 1+2 and Cohort 5, respectively). Steroid and tocilizumab were used in approximately twice as many patients in Cohort 5 compared with Cohorts 1+2, but Cohort 5 was associated with lower cumulative steroid use and higher cumulative tocilizumab use. A similar peak of CD8 T cells was observed in both groups, but a higher naive T-cell peak was observed in Cohort 5 (16.80% vs 31.35%). Regarding product characteristics, the transduction rate was also slightly higher in Cohort 5.

### Discussion

Debulking treatments prior to CAR T-cell therapy may be needed in some patients in clinical practice to limit disease progression or reduce tumor burden during the CAR T-cell manufacturing process. Further, evidence from ZUMA-1 Cohorts 1+2 suggested that lower tumor burden prior to axi-cel infusion was associated with better efficacy and safety outcome. Thus, understanding the impact of debulking on the outcomes of patients treated with axi-cel is important to optimize the management of patients undergoing CAR T-cell treatment. The analysis of Cohort 5 from the ZUMA-1 study aimed to provide some clarity on these questions.

Based on unmatched comparisons, the incidences of Grade≥3 cytokine release syndrome and neurologic events were lower in Cohort 5 (at time of primary analysis) than in ZUMA-1 Cohorts 1+2. The median time to cytokine release syndrome onset was two days in both Cohort 5 and 1+2, and the median duration of cytokine release syndrome was also consistent between the two studies at eight days in both cases. Neurologic events developed more slowly and resolved in a similar time frame in Cohort 5 (median time to onset, 8 days; median duration, 12 days) compared with Cohorts 1+2 (median time to onset, 5 days; median duration, 13 days). Lower incidences of grade≥3 cytokine release syndrome and neurologic events in Cohort 5 versus Cohorts 1+2 may have been influenced by a number of factors, including lower baseline tumor burden, lower CAR T-cell expansion for patients in Cohort 5, the use of prophylactic levetiracetam, or greater clinical experience among treatment teams in managing these toxicities. Additionally, corticosteroid use was higher in Cohort 5 (52% vs 27% in Cohorts 1+2), which may reflect greater confidence among investigators in using corticosteroids with CAR T-cell therapy. In general, the safety profile was similar in Cohort 5 compared with Cohorts 1+2, but there were some hematologic adverse events that were more frequent and more severe in Cohort 5, such as prolonged neutropenia, prolonged thrombocytopenia, and infections, with similar trends persisting through the 24-month follow-up. Finally, a similar incidence of deaths due to adverse events was observed in Cohort 5 and Cohorts 1+2.

The debulking regimens used in Cohort 5 did not appear to have a negative impact on efficacy outcomes. Although the objective response rate in the 24-month analysis of ZUMA-1 Cohort 5 was numerically lower than in the 24-month analysis of ZUMA-1 Cohorts 1+2 (72% versus 83%), the median duration of response in ZUMA-1 Cohort 5 was 25.2 months, whereas the median duration of response in ZUMA-1 Cohorts 1+2 was 11.1 months. The complete response rates in the primary analyses of Cohort 5 and Cohorts 1+2 were comparable (54% vs 52%). The median progression-free survival was 3.1 months for Cohort 5 and 5.9 months for Cohorts 1+2, and the 24-month estimated overall survival was 48% for Cohort 5 and 50.5% for Cohorts 1+2. The usefulness of bridging therapy remains controversial, with contradictory studies recently published. In a recent publication including single-institution real-world evidence of the use of radiotherapy and other bridging therapies in patients with LBCL undergoing CAR T-cell therapy, authors stated that no significant survival or safety differences were observed between the groups that did or did not receive bridging therapy. A separate retrospective study found no significance difference in rates of cytokine release syndrome and neurologic events between patients treated with axi-cel who achieved complete metabolic response with bridging therapy versus those with stable response, partial response, or progressive disease. In addition, no significant progression-free survival differences were found among these patients after a median follow-up of 26 months. In contrast, another study showed that complete or partial response to bridging therapy may reduce disease progression and death in patients treated with axi-cel or tisagenlecleucel; although, this effect seems to be more relevant in patients treated with the latter. Moreover, real-world data show that bridging regimens, particularly radiotherapy or rituximab-bendamustine-polatuzumab, do not have a negative impact on the safety or efficacy of axi-cel or other CAR T-cell therapy.

Subgroup analyses by type of debulking therapy yielded small sample sizes and should be interpreted with caution; observations by type of debulking therapy at time of primary analysis could be hypothesis-generating and will need to be validated in larger studies. In general, outcomes were more favorable for patients who received debulking chemotherapy regimens compared with patients who received radiotherapy only. Serious TEAEs were more common among patients who received chemotherapy versus radiotherapy only, but infections occurred at greater frequency among those who received radiotherapy versus chemotherapy regimens. In contrast, radiotherapy was associated with better efficacy outcomes and similar toxicity levels compared with systemic bridging therapy or no bridging therapy in a study of patients with LBCL who received commercial axi-cel. Here, median duration of response and progression-free survival were notably longer among patients who received debulking chemotherapy versus radiotherapy, with ongoing response rates that were 2.5 times higher in the former group. Despite the differences between our results and real-world experience, the use of debulking therapy did not negatively impact overall efficacy outcomes. It is important to note that bridging therapy should not be considered a definitive therapy. Despite observing complete responses to bridging therapy (like 2 patients in this study), CAR T cells should be administered regardless of the result of bridging therapy if the clinical intent is to treat the patient with CAR T-cell therapy. This is supported by recent evidence demonstrating promising efficacy and safety outcomes among such patients.

In conclusion, the debulking regimens used in Cohort 5 reduced tumor burden prior to axi-cel infusion and demonstrated the feasibility of debulking prior to CAR T-cell therapy in a prospective cohort. However, given the incidence of additional adverse events beyond cytokine release syndrome and neurologic events, the debulking strategies used in Cohort 5 did not appear to improve the overall benefit/risk profile of axi-cel. It is possible that debulking would have had a more favorable overall impact in a patient population with higher tumor burden prior to debulking, which is consistent with the findings that tumor burden impacts outcomes in third-line or later treatment of R/R LBCL.

TABLE 39
Debulking therapy regimens.

| Type | Proposed Regimen^{a} | Timing/Washout |
|---|---|---|
| R-CHOP | Rituximab 375 mg/m² Day 1 | Should have been |
| | Doxorubicin 50 mg/m² Day 1 | administered after |
| | Prednisone 100 mg Day 1 through Day 5 | leukapheresis/enrollment |
| | Cyclophosphamide 750 mg/m² Day 1 | and should have been |
| | Vincristine 1.4 mg/m² Day 1 | completed at least 14 |
| | | days prior to the start of |
| R-ICE | Rituximab 375 mg/m² Day 1 | lymphodepleting |
| | Ifosfamide 5 g/m² 24 h-CI Day 2 | chemotherapy |
| | Carboplatin AUC5 Day 2 maximum dose 800 mg | |
| | Etoposide 100 mg/m²/d Days 1 through Day 3 | |
| R-GEMOX | Rituximab 375 mg/m² Day 1 | |
| | Gemcitabine 1000 mg/m² Day 2 | |
| | Oxaliplatin 100 mg/m² Day 2 | |
| R-GDP | Rituximab 375 mg/m² Day 1 (or Day 8) | |
| | Gemcitabine 1000 mg/m² on Day 1 and Day 8 | |
| | Dexamethasone 40 mg on Day 1 through Day 4 | |
| | Cisplatin 75 mg/m² on Day 1 | |
| | (or carboplatin AUC5 on Day 1) | |
| Radiotherapy^{b} | Per local standard up to 20 to 30 Gy | Should have been |
| | | administered after |
| | | leukapheresis/enrollment |
| | | and should have been |
| | | completed at least 5 days |
| | | prior to the start of |
| | | lymphodepleting |
| | | chemotherapy |

^{a}Other debulking treatment options may have been used but had to be discussed with the medical monitor. Supportive care with hydration, antiemetic, mesna, growth factor support, and tumor lysis prophylaxis according to local standard may have been used. More than one cycle was allowed.
^{b}At least one target lesion should have remained outside of the radiation field to allow for tumor measurements.
AUC5, area under the curve value of 5 mg/mL/min;
CI, continuous infusions;
R-CHOP, rituximab, cyclophosphamide, doxorubicin, vincristine, and prednisone;
R-GDP, rituximab, gemcitabine, dexamethasone, and cisplatin;
R-GEMOX, rituximab, gemcitabine, and oxaliplatin;
R-ICE, rituximab, ifosfamide, carboplatin, and etoposide.

TABLE 40
Patient and disease characteristics at baseline.

| | Overall^{a} | |
|---|---|---|
| Parameter | (N = 50) | |
| Disease type, n (%) | | |
| DLBCL | 36 | (72) |
| TFL | 7 | (14) |
| HGBCL | 7 | (14) |
| Age | | |
| Median (range), years | 57.5 | (29-74) |
| ≥65 years, n (%) | 15 | (30) |
| Male sex, n (%) | 36 | (72) |
| ECOG performance status score of 1, n (%) | 23 | (46) |
| Disease stage, n (%) | | |
| I or II | 13 | (26) |
| III or IV | 37 | (74) |
| IPI score, n (%) | | |
| 0-2 | 25 | (50) |
| 3-4 | 25 | (50) |
| Number of prior lines of chemotherapy, n (%) | | |
| 1 | 4 | (8) |
| 2 | 26 | (52) |
| 3 | 16 | (32) |
| 4 | 2 | (4) |
| 5 | 2 | (4) |
| Prior autologous SCT, n (%) | 16 | (32) |
| PD as best response to most recent | 20 | (40) |
| chemotherapy, n (%) | | |
| Median (range) tumor burden by SPD, mm² | 1652 | (0-36,409) |
| Median (range) LDH, U/L | 262 | (225-479) |
| Median (range) ferritin, ng/mL | 602 | (35-6646) |
| Refractory subgroup, n (%) | | |
| Primary refractory | 4 | (8) |
| Refractory ≥ 2nd-line therapy | 24 | (48) |
| Relapsed ≥ 2nd-line therapy | 6 | (12) |
| Relapsed post-ASCT | 12 | (24) |
| Missing | 4 | (8) |
| Any debulking therapy, n (%) | 48 | (96) |
| R-GEMOX | 9 | (18) |
| R-ICE | 9 | (18) |
| R-GDP | 8 | (16) |
| R-CHOP | 2 | (4) |
| Other^{b} | 7 | (14) |
| Radiotherapy only | 15 | (30) |

Medications onset during retreatment period are excluded.
^{a}All patients treated with at least 1 × 10⁶ CAR T cells/kg.
^{b}Other debulking therapies include rituximab and dexamethasone (n = 2); bendamustine, rituximab, and prednisone (n = 1); bridging chemotherapy, rituximab, and bendamustine (n = 1); R-GDP without cisplatin (n = 1); dexamethasone, methylprednisolone, prednisone, and IGEV (ifosfamide, gemcitabine, vinorelbine, and prednisolone) (n = 1); and prednisone (n = 1).
ASCT, autologous stem cell transplant; DLBCL, diffuse large B-cell lymphoma; ECOG, Eastern Cooperative Oncology Group; HGBCL, high-grade B-cell lymphoma; IPI, International Prognostic Index; LDH, lactate dehydrogenase; PD, progresive disease; SPD, sum of the products of diameters; R-CHOP, rituximab, cyclophoshamide, doxorubicin, vincristine, and prednisone; R-GDP, rituximab, gemcitabine, dexamethasone, and cisplatin; R-GEMOX, rituximab, gemcitabine, and oxaliplatin; R-ICE, rituximab, ifosfamide, carboplatin, and etoposide; TFL, transformed follicular lymphoma.

TABLE 41
Treatment-emergent adverse events of any grade and grade ≥3 adverse
events occurring in ≥15% of patients (primary analysis).

| | | |
|---|---|---|
| n (%) | Any | Any grade ≥3 |

| | | | | |
|---|---|---|---|---|
| Any | 50 | (100) | 50 | (100) |
| Pyrexia | 43 | (86) | 7 | (14) |
| Hypotension | 26 | (52) | 3 | (6) |
| Neutrophil count decreased | 25 | (50) | 24 | (48) |
| Anemia | 19 | (38) | 15 | (30) |
| Headache | 17 | (34) | 0 | (0) |
| Platelet count decreased | 17 | (34) | 14 | (28) |
| Neutropenia | 16 | (32) | 15 | (30) |
| Chills | 14 | (28) | 0 | (0) |
| Tremor | 14 | (28) | 1 | (2) |
| White blood cell count decreased | 13 | (26) | 13 | (26) |
| Fatigue | 12 | (24) | 1 | (2) |
| Nausea | 12 | (24) | 0 | (0) |
| Diarrhea | 11 | (22) | 0 | (0) |
| Hypokalemia | 10 | (20) | 2 | (4) |
| Thrombocytopenia | 9 | (18) | 9 | (18) |
| Aphasia | 9 | (18) | 3 | (6) |
| Lymphocyte count decreased | 8 | (16) | 8 | (16) |
| Leukopenia | 8 | (16) | 7 | (14) |
| Confusional state | 8 | (16) | 2 | (4) |
| Constipation | 8 | (16) | 0 | (0) |
| Dizziness | 8 | (16) | 0 | (0) |

TABLE 42
Treatment-emergent adverse events by debulking regimen (primary analysis)

| | | | | | Other debulking | | | | | | | |
|---|---|---|---|---|---|---|---|---|---|---|---|---|
| | R-ICE/R-GDP | | | | chemotherapies | | | | Radiotherapy only | | | |
| | (n = 17) | | | | (n = 17) | | | | (n = 14) | | | |
| | Any | | | | Any | | | | Any | | | |
| n (%) | grade | | Grade ≥3 | | grade | | Grade ≥3 | | grade | | Grade ≥3 | |
| Any TEAE | 17 | (100) | 17 | (100) | 17 | (100) | 17 | (100) | 14 | (100) | 14 | (100) |
| Serious TEAE | 11 | (65) | 11 | (65) | 8 | (47) | 6 | (35) | 6 | (43) | 4 | (29) |
| CRS | 17 | (100) | 0 | | 11 | (65) | 1 | (6) | 14 | (100) | 0 | |
| NE | 9 | (53) | 2 | (12) | 9 | (53) | 3 | (18) | 8 | (57) | 1 | (7) |
| Infection | 6 | (35) | 5 | (29) | 5 | (29) | 1 | (6) | 7 | (50) | 2 | (14) |

| | | | | | | | | | |
|---|---|---|---|---|---|---|---|---|---|
| Hypogamma- | 2 | (12) | 0 | 1 | (6) | 0 | 1 | (7) | 0 |

globulinemia

CRS, cytokine release syndrome; NE, neurologic event; R-GDP, rituximab, gemcitabine, dexamethasone, and cisplatin; R-ICE, rituximab, ifosfamide, carboplatin, and etoposide; TEAE, treatment-emergent adverse event.

TABLE 43
Incidence, severity, onset, and duration of cytokine
release syndrome and neurologic events.

| | Overall | |
|---|---|---|
| TEAE | (N = 50) | |
| Cytokine Release Syndrome | | |
| Any, n (%) | 43 | (86) |
| Grade 1, n (%) | 19 | (38) |
| Grade 2, n (%) | 23 | (46) |
| Grade 3, n (%) | 0 | |
| Grade 4, n (%) | 1 | (2) |
| Grade 5, n (%) | 0 | |
| Grade ≥3, n (%) | 1 | (2) |
| Median (range) time to onset of any-grade | 2 | (1-9) |
| cytokine release syndrome, days | | |
| Median (range) duration, days | 8 | (1-19) |
| Neurologic Events | | |
| Any, n (%) | 28 | (56) |
| Grade 1, n (%) | 13 | (26) |
| Grade 2, n (%) | 9 | (18) |
| Grade 3, n (%) | 5 | (10) |
| Grade 4, n (%) | 1 | (2) |
| Grade 5, n (%) | 0 | |
| Grade ≥3, n (%) | 6 | (12) |
| Median (range) time to onset of any-grade | 8 | (1-17) |
| neurologic events, days | | |
| Median (range) duration, days | 12 | (1-99) |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

1. A method of predicting a likelihood of a response to a cell therapy product in a patient in need thereof comprising:
quantifying a gene expression level of at least one gene selected from the group consisting of CD19, MS4A1, TNFRSF17, BLK, FCRL2, FAM30A, PNOC, SPIB, TCL1A, BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2; and
determining the likelihood of the response to the cell therapy product in the patient at least in part from the gene expression level,
wherein an increase in the gene expression level of the at least one gene as compared to a control value is indicative of an increased likelihood of a response or of a reduced likelihood of a response as compared to a predetermined likelihood of response rate, and
wherein the gene expression level is quantified from a patient sample, and the patient sample is collected from the patient prior to treatment with the cell therapy product.

2. The method of claim 1, wherein the at least one gene is selected from the group consisting of CD19, MS4A1, TNFRSF17, BLK, FCRL2, FAM30A, PNOC, SPIB, and TCL1A, and wherein an increase in the gene expression level of the at least one gene as compared to a control value is indicative of an increased likelihood of a response as compared to a predetermined likelihood of response rate.

3. The method of claim 1, wherein the at least one gene is selected from the group consisting of CD19, MS4A1, and TNFRSF17, and wherein an increase in the gene expression level of the at least one gene as compared to a control value is indicative of an increased likelihood of a response as compared to a predetermined likelihood of response rate.

4. The method of claim 3, wherein the at least one gene exhibits an increase of at least 20% in the expression level of CD19 versus a control expression level of CD19, an increase of at least 40% in the expression level of MS4A1 versus a control expression level of MS4A1, and an increase of at least 60% in the expression level of TNFRSF17 versus a control expression level of TNFRSF17, is indicative of an increased likelihood of a response as compared to a predetermined likelihood of response rate.

5. The method of claim 1, wherein the at least one gene is selected from the group consisting of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2, and wherein an increase in the gene expression level of the at least one gene as compared to a control value is indicative of a decreased likelihood of a response as compared to a predetermined likelihood of response rate.

6-15. (canceled)

16. A method for treating a malignancy in a patient comprising:
quantifying a gene expression level of at least one gene selected from the group consisting of CD19, MS4A1, TNFRSF17, BLK, FCRL2, FAM30A, PNOC, SPIB, TCL1A, BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2;
determining whether the patient should be administered an effective dose of a cell therapy product as a second-line therapy, or an effective dose of a cell therapy product as a third-line therapy at least in part from the quantifying the gene expression level of at least one gene; and
administering the effective dose of the cell therapy product as a second-line therapy or as a third-line therapy based on the determining step,
wherein the gene expression level is quantified from a patient sample, and the patient sample is collected from the patient prior to treatment with the cell therapy product,
wherein the patient is administered the effective dose of the cell therapy product as a second-line therapy if the gene expression level of the at least one of CD19, MS4A1, TNFRSF17, BLK, FCRL2, FAM30A, PNOC, SPIB, and TCL1A is at or above a control value for the at least one gene, or
wherein the patient is administered the effective dose of the cell therapy product as a third-line therapy if the gene expression level of the at least one of CD19, MS4A1, TNFRSF17, BLK, FCRL2, FAM30A, PNOC, SPIB, and TCL1A is at or below a control value for the at least one gene, or
wherein the patient is administered the effective dose of the cell therapy product as a second-line therapy if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or below a control value for the at least one gene, or
wherein the patient is administered the effective dose of the cell therapy product as a third-line therapy if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or above a control value for the at least one gene.

17. The method of claim 16, wherein if the gene expression level of the at least one of CD19, MS4A1, TNFRSF17, BLK, FCRL2, FAM30A, PNOC, SPIB, and TCL1A is below a control value for the at least one gene, or if the gene expression level of the at least one of BNIP3L, MXI1, ADM, PLOD2, P4HA1, ALDOC, SLC2A1, PDK1, P4HA2, BNIP3, NOS2, IL21R, KIR2DL3, KIR3DL1, and KIR3DL2 is at or above a control value for the at least one gene, then the patient is administered a second-line course of therapy for the malignancy which does not comprise cell therapy.

18. The method of claim 16, wherein the at least one gene is selected from the group consisting of CD19, MS4A1, TNFRSF17, BLK, FCRL2, FAM30A, PNOC, SPIB, and TCL1A.

19. The method of claim 18, wherein the at least one gene is selected from the group consisting of CD19, MS4A1, and TNFRSF17.

20. The method of claim 16, wherein the cell therapy product is CAR T or TCR T cell therapy that recognizes a target antigen.

21. The method of claim 20, wherein the cell therapy product is autologous or allogeneic.

22. The method of claim 20, wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gp120), GPC3 (Glypican 3), as well as any derivate or variant of these antigens.

23. The method of claim 20, wherein the cell therapy product expresses a chimeric antigen receptor comprising a CD28 co-stimulatory domain.

24. The method of claim 16, wherein the patient has been diagnosed with a cancer/tumor selected from the group consisting of a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

25. The method of claim 24, wherein the cancer is (relapsed or refractory) diffuse large B-cell lymphoma (DLBCL) not otherwise specified, primary mediastinal large B-cell lymphoma, high grade B-cell lymphoma (HGBL), DLBCL arising from follicular lymphoma, or mantle cell lymphoma.

26. The method of claim 16, wherein the cell therapy product is selected from axicabtagene ciloleucel, brexucabtagene autoleucel, tisagenlecleucel, lisocabtagene maraleucel, and bb2121.

27. The method of claim 16, wherein the patient sample is a tumor biopsy.
